(19) United States
(12) Patent Application Publication
(10) Pub. No.: US 2013/0164325 A1
(43) Pub. Date: Jun. 27, 2013

(54) CERAMIDE-LIKE GLYCOLIPID-ASSOCIATED BACTERIAL VACCINES AND USES THEREOF

(71) Applicants: Steven A. Porcelli, Bronx, NY (US); Manjunatha M. Venkataswamy, Bronx, NY (US)

(72) Inventors: Steven A. Porcelli, Bronx, NY (US); Manjunatha M. Venkataswamy, Bronx, NY (US)

(73) Assignee: Albert Einstein College of Medicine of Yeshiva University, Bronx, NY (US)

(21) Appl. No.: 13/808,495

(22) PCT Filed: Jul. 6, 2011
(86) PCT No.: PCT/US11/43057

§ 371 Date: Mar. 18, 2013

Related U.S. Application Data
(60) Provisional application No. 61/362,292, filed on Jul. 7, 2010.
(60) Provisional application No. 61/364,335, filed on Jul. 14, 2010.

(51) Int. Cl. A61K 39/39; A61K 39/21; A61K 39/04

(52) U.S. Cl. CPC A61K 39/39; A61K 39/04; A61K 39/21
USPC 424/197.11; 435/252.1; 435/253.1; 435/252.8; 435/252.3

(57) ABSTRACT

The invention is directed compositions and methods related to bacterial cells that are physically associated with ceramide-like glycolipids for use as antigen carriers for heterologous antigens. The invention further relates to methods of incorporating ceramide-like glycolipid to bacterial cell walls. The compositions and methods of the present invention are useful for the prevention and treatment of diseases.

### STATEMENT AS TO FEDERALLY-SPONSORED RESEARCH

This research was funded in part by National Institutes of Health/National Institute of Allergy and Infectious Diseases grants P01-AI063537 and R01-AI45889. Accordingly, the United States Government has certain interest and rights to this invention.

### BACKGROUND OF THE INVENTION

1. Field of the Invention

The invention generally relates to the field of immunology.

2. Background Art

Mycobacterium are known to cause serious diseases in mammals, e.g., tuberculosis, Hansen's disease, leprosy, pulmonary disease resembling tuberculosis, lymphadenitis, skin disease, or disseminated disease. A third of the world's population is infected with Mycobacterium tuberculosis, and 2 million people die from tuberculosis (TB) every year even though the bacille Calmette Guérin (BCG) vaccine has been available for more than 75 years. Hoft D F, Lancet 372: 164-175 (2008). Tuberculosis is currently the second highest cause of death from an infectious disease worldwide, after HIV/AIDS. Young D B et al., Journal of Clinical Investigation 118: 1255-1265 (2008).

Several studies suggest that both MHC class I- and II-restricted T cells are required for effective control of M. tuberculosis infection. Mogues T et al., J Exp Med 193: 271-280 (2001) and Flynn J L et al., Proc Natl Acad Sci USA 89: 12013-12017 (1992). However, mice that are deficient in the lipid-antigen presenting molecule, CD1d, are not more susceptible than wild-type mice to M. tuberculosis infection, indicating that CD1d-restricted NKT cells are not absolutely required for protective immunity. Behar S M et al., J Exp Med 189: 1973-1980 (1999). Natural killer T (NKT) cells represent a subset of T lymphocytes expressing both T-cell receptor and NK-cell receptor, and play a role in bridging innate immunity to adaptive immunity. Kronenberg M and Gapin L, Nat Rev Immunol 2: 557-568 (2002). Upon activation, NKT cells can have a pronounced impact on early and delayed immunity to various pathogens, including L. monocytogenes, M. tuberculosis and Leishmania major. Kronenberg (2002); Behar S M and Porcelli S A, Curr Top Microbiol Immunol 314: 215-250 (2007); Emoto M. et al., Eur J Immunol 29: 650-659 (1999); Ishikawa H et al., Int Immunol 12: 1267-1274 (2000); and Ranson T et al., J Immunol 175: 1137-1144 (2005). NKT cell activation has been reported to lead to enhanced CD4 and CD8 T cell responses, and to induce dendritic cell maturation. Nishimura T et al., Int Immunol 12: 987-994 (2000) and Silk J D et al., J Clin Invest 114: 1800-1811 (2004).

Unlike conventional T cells that recognize MHC-bound peptides, NKT cells are specific for lipid antigens presented by the MHC class I-like protein CD1d. Several glycolipid antigens, including self-derived and bacterial-derived glycolipids, which can be presented by CD1d to activate NKT cells, have been identified to date. Tsuji M. Cell Mol Life Sci 63: 1889-1898 (2006). NKT cells that have T-cell receptors with invariant Vα14-Jα18 rearrangements (iNKT cells) possess reactivity to a glycosphingolipid, α-galactosylceramide (αGalCer), when presented by CD1d. Kronenberg M and Gapin L, Nat Rev Immunol 2: 557-568 (2002); Kronenberg M, Annu Rev Immunol 23: 877-900 (2005). Recent studies have shown that vaccines against Plasmodia, Leishmania donovanii, Listeria monocytogenes and HIV could be improved by activating iNKT cells through co-administration of αGalCer as an adjuvant. Gonzalez-Aseguinolaza G et al., J Exp Med 195: 617-624 (2002); Dondji B et al., European Journal of Immunology 38: 706-719 (2008); Huang Y X et al., Vaccine 26: 1807-1816 (2008); and Enomoto N et al., FEMS Immunol Med Microbiol 51: 350-362 (2007).

As a therapeutic, αGalCer has been shown to reduce malarial parasite load in mice and prolong the survival of M. tuberculosis infected mice. Gonzalez-Aseguinolaza G et al., Proc Natl Acad Sci USA 97: 8461-8466 (2000); Chackerian A et al., Infection and Immunity 70: 6302-6309 (2002). Thus, although CD1d-restricted T cells are not absolutely required for optimum immunity, their specific activation enhances host resistance to infectious diseases.

A single injection of αGalCer in mice induces a cytokine storm in the serum resulting in secretion of IFNγ, IL-12 and IL-4. Fujii S et al., Immunol Rev 220: 183-198 (2007). Stimulation of CD1d-restricted iNKT cells by αGalCer also leads to rapid activation of NK cells, dendritic cells, B cells, and conventional T cells. Nishimura T et al., Int Immunol 12: 987-994 (2000); Kitamura H et al., J Exp Med 189: 1121-1128 (1999); Fujii S et al., J Exp Med 198: 267-279 (2003). iNKT cells produce large amounts of IFNγ and the production requires direct contact between iNKT cells and DCs through CD40-CD40 ligand interactions. Nishimura T et al., Int Immunol 12: 987-994 (2000). IFNγ produced by iNKT cells has been shown to have a critical role in the antimetastatic effect of αGalCer in murine tumor models. Hayakawa Y et al., Eur J Immunol 31: 1720-1727 (2001); Smyth M J et al., Blood 99: 1259-1266 (2002). Thus, it has been proposed that activation of iNKT cells can modulate adaptive immune responses by influencing the early cytokine environment.

Recently, a C-glycoside analogue of αGalCer known as the α-C-GalCer has been established as a predominant Th1 skewing compound which has a superior anti-tumor and anti-malarial activity as compared to αGalCer in mice. This compound also induces higher levels of Th1 cytokines IL-12 and IFNγ in mice. Schmieg J et al., Journal of Experimental Medicine 198: 1631-1641 (2003). It has been established that these two cytokines, IL-12 and IFNγ, are essential for control of TB in mice and humans. Freidag B L et al., Infect Immun 68: 2948-2953 (2000).

Very few studies exist on the use of adjuvants with BCG vaccine in the mouse model against tuberculosis. One such study reported an enhanced protection against M. tuberculosis challenge when CpG ODN was used along with BCG vaccination. Freidag B L et al., Infect Immun 68: 2948-2953 (2000). Most of the earlier studies on the adjuvant effect of αGalCer with vaccines against various infectious diseases have utilized separate co-administration of αGalCer with the respective vaccine in order to harness its adjuvant activity. Gonzalez-Aseguinolaza G et al. (2002); Dondji B et al. (2008); Huang Y X et al. (2008); and Enomoto N et al. (2007). In PCT/US2010/020531, Porcelli et al., filed Jan. 8, 2010, the inventors disclosed bacterial vaccines with cell wall-associated ceramide-like glycolipids and uses thereof.

Natural surface lipids were removed from the Mycobacterium tuberculosis (MTB) cell wall using petroleum ether extraction (delipidated MTB) in Indrigo et al., Microbiology. 148:1991-1998 (2002), resulting in decreased bacterial survivial in macrophages. The bacterial survivial within macrophages was restored upon reconstitution of the bacteria with purified trehalose 6,6′-dimycolate (TDM), a natural lipid of the MTB cell wall, in petroleum ether. See Indrigo et al., Microbiology. 148:1991-1998 (2002); Rao, et al. J Exp Med. 201(4):535-43 (2005). Methods disclosed in PCT/US2010/020531 for incorporating glycolipids into live mycobacteria include culturing mycobacterial cells and glycolipid in culture medium under conditions that require low concentrations of nonionic detergent, e.g., 0.05% TWEEN 80 or Tyloxapol. Such methods are difficult to standardize and inefficient as they require a high amount of glycolipid to be added to the culture medium.

There remains a need for effective compositions and vaccines for enhancing immune responses to heterologous antigens. Furthermore, there remains a need for methods of preparing bacterial vaccines with cell wall-associated ceramide-like glycolipids that can be used as adjuvants and/or heterologous antigen carriers.

### SUMMARY OF THE INVENTION

The present invention is directed to a modified bacterium comprising a bacterial cell, a heterologous antigen, and a ceramide-like glycolipid, wherein the ceramide-like glycolipid is physically associated with the bacterial cell. In a further embodiment, the ceramide-like glycolipid comprises a glycosylceramide or an α-galactosylceramide or analogs thereof.

In one embodiment, the glycosylceramide or analog thereof comprises Formula I:

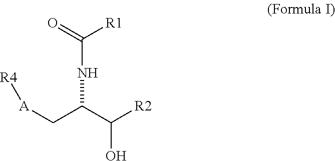

wherein R1 is a linear or branched C₁-C₂₇ alkane or C₂-C₂₇ alkene; or R1 is —C(OH)—R3 wherein R3 is a linear or branched C₁-C₂₆ alkane or C₂-C₂₆ alkene; or R1 is a C₆-C₂₇ alkane or alkene wherein (i) the C₆-C₂₇ alkane or alkene is substituted with a C₅-C₁₅ cycloalkane, C₅-C₁₅ cycloalkene, heterocycle, or aromatic ring or (ii) the C₆-C₂₇ alkane or alkene includes, within the C₆-C₂₇ alkyl or alkenyl chain, a C₅-C₁₅ cycloalkane, C₅-C₁₅ cycloalkene, heterocycle, or aromatic ring;

R2 is one of the following (a)-(e):

—CH₂(CH₂)ₓCH₃, (a)

—CH(OH)(CH₂)ₓCH₃, (b)

—CH(OH)(CH₂)ₓCH(CH₃)₂, (c)

—CH═CH(CH₂)ₓCH₃, (d)

—CH(OH)(CH₂)ₓCH(CH₃)CH₂CH₃, (e)

wherein X is an integer ranging from 4-17;

R4 is an α-linked or a β-linked monosaccharide, or when R1 is a linear or branched C₁-C₂₇ alkane, R4 is:

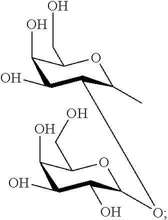

and A is O or —CH₂.

In one embodiment, the α-galactosylceramide or analog thereof comprises Formula II:

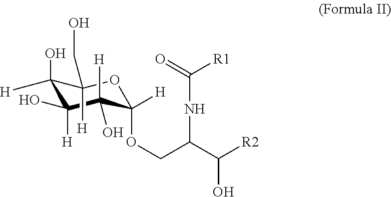

wherein

R1 is a linear or branched C₁-C₂₇ alkane or C₂-C₂₇ alkene; or R1 is —C(OH)—R3 wherein R3 is linear or branched C₁-C₂₆ alkane or C₂-C₂₆ alkene; and

R2 is one of the following (a)-(e):

—CH₂(CH₂)ₓCH₃, (a)

—CH(OH)(CH₂)ₓCH₃, (b)

—CH(OH)(CH₂)ₓCH(CH₃)₂, (c)

—CH═CH(CH₂)ₓCH₃, (d)

—CH(OH)(CH₂)ₓCH(CH₃)CH₂CH₃, (e)

wherein X is an integer ranging from 4-17.

In one embodiment, the α-galactosylceramide or analog thereof comprises Formula III:

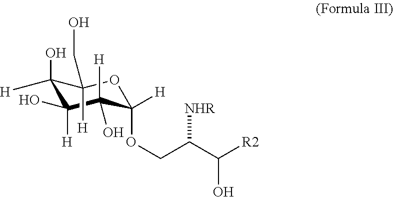

wherein R is H or —C(O)R1, wherein R1 is a linear or branched C₁-C₂₇ alkane or C₂-C₂₇ alkene; or R1 is —C(OH)—R3 wherein R3 is a linear or branched C₁-C₂₆ alkane or C₂-C₂₆ alkene; or R1 is a C₆-C₂₇ alkane or alkene wherein (i) the C₆-C₂₇ alkane or alkene is substituted with a C₅-C₁₅ cycloalkane, C₅-C₁₅ cycloalkene, heterocycle, or aromatic ring or (ii) the C₆-C₂₇ alkane or alkene includes, within the C₆-C₂₇ alkyl or alkenyl chain, a C₅-C₁₅ cycloalkane, C₅-C₁₅ cycloalkene, heterocycle, or aromatic ring; or R1 is a —(CH₂)ₙR5, wherein n is an integer ranging from 0-5, and R5 is —C(O)OC₂H₅, an optionally substituted C₅-C₁₅ cycloalkane, an optionally substituted aromatic ring, or an aralkyl, and

R2 is one of the following (a)-(e):

—CH₂(CH₂)ₓCH₃, (a)

—CH(OH)(CH₂)ₓCH₃, (b)

—CH(OH)(CH₂)ₓCH(CH₃)₂, (c)

—CH═CH(CH₂)ₓCH₃, (d)

—CH(OH)(CH₂)ₓCH(CH₃)CH₂CH₃, (e)

wherein X is an integer ranging from 4-17.

In one embodiment, a ceramide-like glycolipid is incorporated into the cell wall of a bacterial cell. In a further embodiment, the bacterial cell is selected from the group consisting of a mycobacterial cell, a Listeria cell, a Salmonella cell, a Yersinia cell, a Francisella cell, and a Legionella cell. In another embodiment, the bacterial cell is live, killed, or attenuated. In another embodiment, the heterologous antigen linked to the surface of the bacterial cell. In another embodiment, the ceramide-like glycolipid is incorporated into the cell wall of a recombinant bacterial cell that comprises a gene the encodes a heterologous antigen.

In another embodiment, the ceramide-like glycolipid is incorporated into the cell wall of a bacterial cell using a method comprising coupling a protecting group, e.g., TMS, to the hydroxyls of the glycolipid and solubolizing the protected glycolipid in a solvent, e.g., petroleum ether, and thereafter suspending a live bacterial cell, e.g., a mycobacterial cell, in the hydroxyl-glycolipid solvent solution, and then evaporating the solvent. In another embodiment, the modified bacterium is produced by said method.

In one embodiment, the modified bacterium enhances antigen-specific CD8 T cell responses against an antigen. In a further embodiment, the antigen is a mycobacterial antigen.

In one aspect of the invention, the heterologous antigen is linked to the surface of said bacterial cell (e.g., an ectopic antigen). In one embodiment, the heterologous antigen is chemically or physically conjugated to the surface of the bacterial cell. In one embodiment, the modified bacterium expresses a heterologous antigen. In a further embodiment, the heterologous antigen is a viral antigen, a bacterial antigen, a fungal antigen, a parasitic antigen, or a tumor specific antigen. In another embodiment, the heterologous antigen is an immunogenic peptide. In certain embodiments, the heterologous antigen is expressed on the surface of said bacterial cell (e.g., an ectopic antigen).

In one embodiment, the bacterial cell is a recombinant bacterial cell. In one embodiment, the heterologous antigen is expressed by the recombinant bacterial cell.

The present invention is also directed to a composition comprising a modified bacterium and a pharmaceutical carrier. In one embodiment, the pharmaceutical carrier is selected from the group consisting of saline, buffered saline, dextrose, water, glycerol, and combinations thereof. In another embodiment, the composition further comprises an adjuvant. In another embodiment, the composition is a vaccine composition.

The present invention is also directed to methods of treating or preventing a disease in an animal, comprising administering to an animal in need of treatment or prevention a modified bacterium. In one embodiment, the modified bacterium is administered in an amount sufficient to alter the progression of the disease. In another embodiment, the modified bacterium is administered in an amount sufficient to induce an immune response in the animal against the disease.

In one embodiment, an immune response is enhanced or modified relative to an immune response produced by a bacterial cell not associated with a ceramide-like glycolipid. In a further embodiment, the immune response is enhanced or modified relative to an immune response produced by a bacterial cell linked to a heterologous antigen not associated with a ceramide-like glycolipid. In one embodiment, the primary response is enhanced. In another embodiment, the secondary immune response is enhanced. In another embodiment, both the primary and secondary immune responses are enhanced. In one embodiment, the disease is selected from the group consisting of a viral disease, a bacterial disease, a fungal disease, a parasitic disease, and a proliferative disease. In a further embodiment, the disease is selected from the group consisting of tuberculosis, pulmonary disease resembling tuberculosis, lymphadenitis, skin disease, disseminated disease, bubonic plague, pneumonic plague, tularemia, Legionairre's disease, anthrax, typhoid fever, paratyphoid fever, foodborne illness, listeriosis, malaria, Human Immunodeficiency Virus (HIV), Simian Immunodeficiency Virus (SIV), Human Papilloma Virus (HPV), Respiratory Syncitial Virus (RSV), influenza, hepatitis (HAV, HBV, and HCV), and cancer.

The present invention is also directed to a method of inducing an immune response against an antigen in an animal, comprising administering to the animal a modified bacterium. In one embodiment, the modified bacterium is administered in an amount sufficient to enhance an antigen-specific CD8 T-cell response or enhance the activity of Natural Killer T (NKT) cells in the animal. In another embodiment, the immune response comprises an antibody response. In another embodiment, the immune response comprises a CD8 T-cell response. In another embodiment, the immune response comprises a CD8 T-cell response and an antibody response.

The present invention is also directed to a method of modulating a CD8 T-cell response to BCG in an animal comprising administering to the animal an effective amount of a modified bacterium.

In one embodiment, the modified bacterium is administered by a route selected from the group consisting of intramuscularly, intravenously, intratracheally, intranasally, transdermally, intradermally, subcutaneously, intraocularly, vaginally, rectally, intraperitoneally, intraintestinally, by inhalation, or by a combination of two or more of said routes.

The present invention is also directed to a kit comprising a modified bacterium. In one embodiment, the modified bacterium is lyophilized. In a further embodiment, the kit comprises a means for administering the modified bacterium.

The present invention is also directed to a method of making a ceramide-like glycolipid/mycobacterial complex comprising (a) coupling a protection group to the hydroxyls of a ceramide-like glycolipid; (b) adding a solvent to the hydroxyl-protected ceramide-like glycolipid of (a) to produce a hydroxyl-glycolipid solvent solution; (c) suspending a mycobacterium in the hydroxyl-glycolipid solvent solution; and (d) evaporating said solvent, thereby making said ceramide-like glycolipid/mycobacterial complex. In one embodiment, the solvent is nonpolar. In certain embodiments, the nonpolar solvent is a hexane, a benzene, a toluene, a diethyl ether, an ethyl acetate, a hydrocarbon mixture, or any combination thereof. In certain embodiments, the solvent is petroleum ether, dimethyl sulfoxide (DMSO), or benzine. In certain embodiments, the protection group is an alcohol protecting group. In one embodiments, the protection group is a Silyl ether, e.g., trimethylsilyl (TMS), tert-butyldimethylsilyl (TBDMS), tert-butyldimethylsilyloxymethyl (TOM), and triisopropylsilyl (TIPS) ethers.

In one embodiment, the invention is directed to a method of producing a vaccine against a heterologous antigen comprising: (a) isolating a ceramide-like glycolipid/mycobacterial complex and (b) adding a pharmaceutical carrier to the isolated complex of (a).

These and other aspects of the invention are described in further detail below.

### BRIEF DESCRIPTION OF THE FIGURES

FIG. 1: shows FACS data for the primary response (PBMC) at day 14 in C57BL/6 mice that were immunized with BCG (BCG-P); BCG/SIV-Gag; or α-C-GalCer modified BCG/SIV-Gag (10⁷ CFU, i.v.).

FIG. 2: shows the increase in Gag-specific CD8+ T primary response in mice immunized with α-C-GalCer modified BCG/SIV-Gag compared to BCG alone or BCG/SIV-Gag (p<0.01 (ANOVA) compared to BCG/SIV-Gag).

FIG. 3: shows FACS data for the secondary response (PBMC) at day 7 post-boost in C57BL/6 mice that were primed with BCG; BCG/SIV-Gag; or α-C-GalCer modified BCG/SIV-Gag (10⁷ CFU, retro-orbital), and twelve weeks later were administered with a suboptimal dose of rAd5/SIV-Gag (10⁷ PFU, i.m.), or sham boost with saline.

FIG. 4: shows the increase in Gag-specific CD8+ T secondary response in mice immunized (primed at day −84) with α-C-GalCer modified BCG/SIV-Gag compared to BCG alone or BCG/SIV-Gag at days 7 and 14 post-Ad5/SIV-Gag boost.

FIG. 5: shows the structures of Ac-KRN700 DB09-5 and TMS-KRN700 DB09-6 in FIG. 5A. The in vivo activation of mouse splenocytes with KRN7000- and TMS-KRN7000-infected BMDCs induced IFNγ production is shown in FIG. 5B. The detected IL-2 (ng/ml) in the supernatant of Bone Marrow-derived Dendritic Cells (BMDC) infected with BCG, BCG/Ac-KRN7000, and BCG/TMS-KRN7000 incubated with an NKT cell hybridoma is shown in FIG. 5C.

### DETAILED DESCRIPTION OF THE INVENTION

The present invention provides compositions, isolated cells, vaccines, and methods, which are useful for enhancing, i.e., eliciting, stimulating or increasing, an immune response, e.g., a primary and/or secondary immune response. Described herein is a modified bacterium comprising a ceramide-like glycolipid physically associated with a bacterial cell, e.g., ceramide-like glycolipids stably incorporated into a bacterial cell wall, e.g., a mycobacterial cell wall. In certain embodiments, the modified bacterium further comprises a heterologous antigen, e.g., a viral antigen, a bacterial antigen, a fungal antigen, a parasitic antigen, or a tumor specific antigen. The ceramide-like glycolipid/bacterial complexes expressing a heterologous antigen of the present invention can enhance an immune response by affecting the activity of CD1d-restricted natural killer T (“NKT”) cells. Described herein are ceramide-like glycolipid/bacterial complexes expressing a heterologous antigen of the invention, which can be used for enhanced priming of an immune response against the heterologous antigen. In certain embodiments, the compositions, e.g., vaccine compositions, of the invention include an α-galactosylceramide or analog thereof incorporated into the cell wall of M. bovis bacille Calmette-Guerin (BCG), which expresses a heterologous antigen, e.g., an immunogenic peptide. The ceramide-like glycolipid/bacterial complexes of the invention can be prepared by coupling a protection group, e.g., tri-methyl silicate (TMS), to the hydroxyls of a ceramide-like glycolipid; adding a solvent, e.g., petroleum ether (PetEther), to the hydroxyl-protected ceramide-like glycolipid to produce a hydroxyl-protected glycolipid solvent solution, thereafter suspending a bacterium, e.g., a mycobacterium, in the hydroxyl-protected glycolipid solvent solution; and evaporating the solvent, thereby making a ceramide-like glycolipid/mycobacterial complex. Ceramide-like glycolipid/bacterial complexes as described herein are useful for stimulating desirable immune responses, for example, immune responses against mycobacterial antigens or heterologous antigens. The immune response can be useful for preventing, treating or ameliorating diseases caused by bacterial pathogens; e.g., mycobacteria, e.g., Mycobacterium tuberculosis, which causes TB in humans; other infectious agents; or tumors.

Advantages of the invention include improved methods for incorporating glycolipids, e.g., ceramide-like glycolipids, into the cell wall of bacteria, e.g., mycobacteria. Advantages also include that administration of the antigen carriers of the invention, e.g., ceramide-like glycolipid/bacterial complexes that carry heterologous antigens, to an animal to enhance priming of an immune response against the heterologous antigen. Advantages also include that the delivery of ceramide-like glycolipid adjuvants directly to the same cells that become infected with a bacteria, e.g., a live attenuated bacteria, allows the focusing of the adjuvant in a way that permits much smaller doses to be used. Thereby reducing local and systemic toxicity and lowering production costs. In addition, physically linking, e.g., direct incorporation, has practical advantages, particularly for vaccines that target populations in the third world where there are delivery and storage issues. Bacteria physically associated, e.g., directly incorporated, with ceramide-like glycolipids, which are lyophilized and then reconstituted should allow for adjuvant activity to be recovered intact. Thus, the lyophilized vaccine could be rehydrated and suspended in the field for administration.

### DEFINITIONS

It is to be noted that the term “a” or “an” entity refers to one or more of that entity; for example, “a vector” is understood to represent one or more vectors. As such, the terms “a” (or “an”), “one or more,” and “at least one” can be used interchangeably herein.

As discussed in more detail below, the present invention includes a glycolipid, typically a ceramide-like glycolipid, e.g., an α-galactosylceramide, also referred to herein as α-GalCer, or an analog thereof, such as α-C-GalCer, physically associated with a bacterial cell, e.g., incorporated into a bacterial cell wall, e.g., a mycobacterial cell wall. In certain embodiments, the ceramide-like glycolipid is physically associated through non-covalent interactions. “Ceramide-like glycolipids,” as referred to herein include glycolipids with α-linked galactose or glucose. Examples of ceramide-like glycolipids are described herein and also can be found, e.g., in Porcelli, U.S. Patent Appl. Publ. No. 2006/0052316, Tsuji, U.S. Patent Appl. Publ. No. 2006/0211856, Jiang, U.S. Patent Appl. Publ. No. 2006/0116331, Hirokazu et al., U.S. Patent Appl. Publ. No. 2006/0074235, Tsuji et al., U.S. Patent Appl. Publ. No. 2005/0192248, Tsuji, U.S. Patent Application No. 2004/0127429, and Tsuji et al., U.S. Patent Application No. 2003/0157135, all of which are incorporated by reference herein in their entireties.

### Vaccines

The term “vaccine” refers to a composition, which when administered to an animal is useful in stimulating an immune response, e.g., against an antigen, e.g., a heterologous antigen. The invention relates to a vaccine composition comprising bacterial cells, e.g., mycobacterial cells, wherein said cells can be killed, live and/or attenuated, for example, BCG, which is a live attenuated bacterial vaccine. Bacterial vaccines, e.g., live bacterial vaccines, killed bacterial vaccines, or attenuated bacterial vaccines are known in the art or can be produced by methods well known to a person of ordinary skill in the art using routine experimentation. A bacterial vaccine of the invention can also include recombinant bacteria, e.g., a recombinant mycobacteria. A bacterial vaccine of the invention can also include bacterial cells that express a heterologous antigen, e.g., a recombinant mycobacterial cell that expresses an infectious agent antigen or tumor antigen.

In certain embodiments, a bacterial cell and a ceramide-like glycolipid are administered with a heterologous antigen. In one embodiment, a bacterial cell is modified, “glycolipid modified” to physically link a glycolipid to the bacterial cell, e.g., a ceramide-like glycolipid is incorporated into the cell wall of a bacterial cell, e.g., a mycobacterial cell. In a further embodiment, the modified bacterium can be used as an antigen carrier, e.g., for delivery of a heterologous antigen.

In certain embodiments, glycolipid modified bacterial cell of the invention is used as an “antigen carrier” for the delivery of a heterologous antigen, e.g., an immunogenic polypeptide. For example, a glycolipid modified bacterial cell, e.g., a recombinant bacterial cell having a ceramide-like glycolipid incorporated into its cell wall can be used as a antigen carrier for the delivery of antigens from another pathogen (e.g., bacterial (e.g., Salmonella, Listeria, Bacillus anthracis, and Shigella antigens), fungal, parasitic (e.g., a malarial antigen from Plasmodium), or viral antigens (e.g., a viral antigen from HIV, SIV, HPV, RSV, influenza or hepatitis (HAV, HBV, and HCV)) or tumor specific antigens.

In one embodiment, modified bacteria of the invention include modified mycobacterial cells, e.g., M. bovis bacille Calmette-Guérin (BCG) cells to which α-GalCer or an analog thereof, e.g., α-C-GalCer, has been stably non-covalently incorporated. BCG is a live attenuated bacterial vaccine. Albert Calmette and Camille Guérin of the Pasteur Institute attenuated mycobacterium related to Mycobacterium bovis, which is closely related to M. tuberculosis, to produce Mycobacterium bovis bacillus Calmette-Guérin (BCG) by growing it in culture medium for 13 years, and monitoring its decrease in virulence in animals through this period. BCG has become one of the most widely used of all vaccines, being both inexpensive and safe. However, the BCG vaccine has had limited effect against the epidemic of TB in the developing world. Doherty T and Anderson P, Clinical Microbio Reviews 18(4):687-702 (2005). In another embodiment, the mycobacterial cells are M. smegmatis cells, which is another nonpathogenic strain of mycobacteria that can be administered to mammals without causing disease.

In addition to modified mycobacterial cells, other modified bacteria of the invention include, without limitation glycolipid modified bacteria derived from Bacillus species (e.g., Bacillus anthracis causing anthrax), Salmonella species (e.g., causing typhoid fever, paratyphoid fever, foodborne illness), Staphylococcus species, Streptococcus species, Listeria species (e.g., causing listeriosis), Shigella species, Yersinia species (e.g., causing bubonic and pneumonic plague), Francisella species (e.g., causing tularemia), and Legionella species (e.g., causing Legionnaire's disease).

In certain embodiments, the modified bacteria of the invention include a bacteria. e.g., a mycobacteria, to which a glycolipid, e.g., a glycolipid that is not a native lipid of the bacterial cell wall, has been stably incorporated. In certain embodiments, the modified bacterial cell of the invention is prepared by coupling a protection group, e.g., tri-methyl silicate (TMS), to the hydroxyls of a ceramide-like glycolipid; adding a solvent, e.g., petroleum ether (PetEther), to the hydroxyl-protected ceramide-like glycolipid to produce a hydroxyl-protected glycolipid solvent solution, thereafter suspending a bacterium in the hydroxyl-protected glycolipid solvent solution; and evaporating the solvent, thereby making a ceramide-like glycolipid/bacterial complex.

The term “antigen” and the related term “antigenic” as used herein refer to a substance that binds specifically to an antibody or to a T-cell receptor.

The term “immunogen” and the related term “immunogenic” as used herein refer to the ability to induce an immune response, including an antibody and/or a cellular immune response in an animal, for example a mammal. It is likely that an immunogen will also be antigenic, but an “antigen,” because of its size or conformation, may not necessarily be an “immunogen.” An “immunogenic composition” induces an immune response in a subject, e.g., antibodies that specifically recognize one or more antigens, contained within that “immunogenic composition.” In certain embodiments, the immunogenic composition of the invention comprises a ceramide-like glycolipid/bacterial complex and a heterologous antigen. In certain embodiments, the immune response involves a primary immune response, e.g., a priming immune response to a modified bacterium expressing the heterologous antigen, and a secondary immune response, e.g., a secondary response post-boost with the heterologous antigen.

The term “immune response” is meant to include an activity of cells of the immune system in response to an antigen or immunogen. Such activities include, but are not limited to production of antibodies, cytotoxicity, lymphocyte proliferation, release of cytokines, inflammation, phagocytosis, antigen presentation, and the like. An immune response which is highly specific to a given antigen or immunogen, e.g., production of specific antibodies or production of specific T lymphocytes is referred to herein as an “adaptive immune response.” An immune response which is not specific to a given antigen, e.g., release of cytokines by NK and NKT cells, is referred to herein an “innate immune response.” Examples of immune responses include an antibody response or a cellular, e.g., cytotoxic response.

The terms “protective immune response” or “therapeutic immune response” refer to an immune response to an immunogen, which in some way prevents or at least partially arrests disease symptoms, side effects or progression. By “protective” is meant that the immune response is induced in a subject animal which has not contracted a disease, where the immune response alleviates, reduces, moderates or, in some cases fully prevents disease symptoms if the animal later contracts or is susceptible to that disease, e.g., exposure to M. tuberculosis. By “therapeutic” is meant that the immune response is induced in a subject animal which has the disease, e.g., a human with tuberculosis, where the immune response alleviates, reduces, moderates, or in some cases fully eliminates disease symptoms. In certain embodiments, the compositions of the invention are used to induce a therapeutic immune response in an animal, e.g., a human, having an infectious disease or cancer.

The term “modulating an immune response” is meant to refer to any way in which a given immune response is increased, decreased, or changed by a composition or treatment relative to the immune response without that composition or treatment. For example, use of an adjuvant, e.g., a modified bacterium expressing a heterologous antigen of the invention, to increase an immune response to an antigen, e.g., the heterologous antigen, is considered modulation of that immune response. Decrease in an immune response, e.g., prevention of autoimmunity, is also a modulation. In addition, changing an immune response, e.g., from a primary TH2 response to a primary TH1 response, is a modulation of an immune response. The present invention provides methods of modulating an immure response by administering to an animal a composition which comprises a modified bacterium, e.g., a bacterial cell with a ceramide-like glycolipid incorporated into its cell wall, e.g., a mycobacterial cell wall, and a heterologous antigen.

The present invention provides compositions and methods useful for enhancing both primary and secondary immune responses. In one aspect, the primary and/or secondary immune response is against a heterologous antigen that is expressed by the modified mycobacteria of the invention. In one aspect, the modified mycobacteria of the invention is used as an antigen carrier for delivery of antigens, e.g., heterologous antigens that enhance an immune response against an infectious agent or tumor.

The term “adjuvant” refers to a material having the ability to (1) alter or increase the immune response to a particular antigen or (2) increase or aid an effect of a pharmacological agent. In certain embodiments, a ceramide-like glycolipid functions as an adjuvant upon simultaneous administration with a bacterial cell, e.g., a BCG, e.g., when the ceramide-like glycolipid is incorporated into the BCG cell wall. In certain embodiments, the ceramide-like glycolipid, e.g., αGalCer or analog thereof, functions as an adjuvant when administered with a bacterial cell and heterologous antigen. In another embodiment, a second adjuvant is included. Other suitable adjuvants include, but are not limited to, LPS derivatives (e.g., monophosphoryl lipid A (MPL)), TLR9 agonists (e.g., CPG ODNS), TLR7/8 agonists (e.g., imiquimod), cytokines and growth factors; bacterial components (e.g., endotoxins, in particular superantigens, exotoxins and cell wall components); aluminum-based salts; calcium-based salts; silica; polynucleotides; toxoids; serum proteins, viruses and virally-derived materials, poisons, venoms, imidazoquiniline compounds, poloxamers, and cationic lipids.

A great variety of materials have been shown to have adjuvant activity through a variety of mechanisms. Any compound which can increase the expression, antigenicity or immunogenicity of an immunogen is a potential adjuvant. Other potential adjuvants of the invention include, but are not limited to: glycolipids; chemokines; compounds that induces the production of cytokines and chemokines; interferons; inert carriers, such as alum, bentonite, latex, and acrylic particles; pluronic block polymers, such as TiterMax® (block copolymer CRL-8941, squalene (a metabolizable oil) and a microparticulate silica stabilizer); depot formers, such as Freunds adjuvant; surface active materials, such as saponin, lysolecithin, retinal, Quil A, liposomes, and pluronic polymer formulations; macrophage stimulators, such as bacterial lipopolysaccharide; alternate pathway complement activators, such as insulin, zymosan, endotoxin, and levamisole; non-ionic surfactants; poly(oxyethylene)-poly(oxypropylene) tri-block copolymers; mLT; MF59™; SAF; Ribi™ adjuvant system; trehalose dimycolate (TDM); cell wall skeleton (CWS); Detox™; QS21; Stimulon™; complete Freund's adjuvant; incomplete Freund's adjuvant; macrophage colony stimulating factor (M-CSF); tumor necrosis factor (TNF); 3-O-deacylated MPL; CpG oligonucleotides; polyoxyethylene ethers, polyoxyethylene esters, and combinations of more than one adjuvant.

In certain embodiments, the adjuvant is a cytokine. A composition of the present invention can comprise one or more cytokines, chemokines, or compounds that induce the production of cytokines and chemokines. Examples include, but are not limited to granulocyte macrophage colony stimulating factor (GM-CSF), granulocyte colony stimulating factor (G-CSF), macrophage colony stimulating factor (M-CSF), colony stimulating factor (CSF), erythropoietin (EPO), interleukin 2 (IL-2), interleukin-3 (IL-3), interleukin 4 (IL-4), interleukin 5 (IL-5), interleukin 6 (IL-6), interleukin 7 (IL-7), interleukin 8 (IL-8), interleukin 10 (IL-10), interleukin 12 (IL-12), interleukin 15 (IL-15), interleukin 18 (IL-18), interferon alpha (IFNα), interferon beta (IFNβ), interferon gamma (IFNγ), interferon omega (IFNω), interferon tau (IFNτ), interferon gamma inducing factor I (IGIF), transforming growth factor beta (TGF-β), RANTES (regulated upon activation, normal T-cell expressed and presumably secreted), macrophage inflammatory proteins (e.g., MIP-1 alpha and MIP-1 beta), Leishmania elongation initiating factor (LEIF), and Flt-3 ligand.

In certain embodiments, compositions of the invention further comprise another component, e.g., a polypeptide with immunological activity. For example, the protein with immunological activity is a costimulatory molecule, such as a toll-like receptor (“TLR”), B7.1 or B7.2. “B7” is used herein to generically refer to either B7.1 or B7.2. A costimulatory molecule, e.g., the extracellular domain of B7-1 (CD80) or B7-2 (CD86) that interacts with CD28 on T- and NK-cells can be administered as an amino terminal fusion to β2-microglobulin incorporated into the structure of a soluble CD1d complex for use in the present invention. See, e.g., WO 9964597, published 16 Dec. 1999. In certain embodiments, incorporation of a costimulatory molecule, e.g., a B7 signaling molecule, with the compositions of the invention allows more effective and prolonged activation of NKT cells by a ceramide-like glycolipid/bacterial complex of the invention.

In other embodiments, the compositions of the invention further comprise additional adjuvant components, e.g., any of the adjuvants described above, such as, LPS derivatives (e.g., MPL), TLR9 agonists (e.g., CPG ODNS), TLR7/8 agonists (e.g., imiquimod), cytokines and growth factors; bacterial components (e.g., endotoxins, in particular superantigens, exotoxins and cell wall components); aluminum-based salts; calcium-based salts; silica; polynucleotides; toxoids; serum proteins, viruses and virally-derived materials, poisons, venoms, imidazoquiniline compounds, poloxamers, cationic lipids, and Toll-like receptor (TLR) agonists. Examples of TLR agonist adjuvants which can be effective, include, but are not limited to: N-acetylmuramyl-L-alanine-D-isoglutamine (MDP), lipopolysaccharides (LPS), genetically modified and/or degraded LPS, alum, glucan, colony stimulating factors (e.g., EPO, GM-CSF, G-CSF, M-CSF, PEGylated G-CSF, SCF, IL-3, IL6, PIXY 321), interferons (e.g., γ-interferon, α-interferon), interleukins (e.g IL-1, IL-2, IL-3, IL-4, IL-5, IL-6, IL-10, IL-12, IL-15, IL-18), saponins (e.g. QS21), monophosphoryl lipid A (MPL), 3 De-O-acylated monophosphoryl lipid A (3D-MPL), unmethylated CpG sequences, 1-methyl tryptophan, arginase inhibitors, cyclophosphamide, antibodies that block immunosuppressive functions (e.g., anti-CTLA4 antibodies), lipids (such as palmitic acid residues), tripalmitoyl-S-glycerylcystein lyseryl-serine (P₃ CSS), and Freund's adjuvant. Alternatively or additionally, compositions of the present invention my further comprise a lymphokine or cytokine that modulates immune cell activation such as transforming growth factor (TGF, e.g., TGFα and TGFβ); α interferons (e.g. IFNα); β interferons (e.g. IFNβ); γ interferons (e.g. IFNγ) or lymphocyte function-associated protein, such as LFA-1 or LFA-3; or an intercellular adhesion molecule. such as ICAM-1 or ICAM-2.

Compositions of the invention can further comprise an immunogenic polypeptide. In certain embodiments, glycolipid modified bacterial cells of the invention can be used as antigen carriers for the delivery of heterologous antigens or immunogens. Heterologous antigens or in immunogens can include, but are not limited to, immunogenic polypeptides. In one embodiment, the immunogenic polypeptide can be expressed by a glycolipid modified recombinant bacterial cell of the invention, e.g., immunogenic polypeptides of heterogous pathogens expressed by recombinant mycobacterial cells with a ceramide-like glycolipid incorporated into the mycobacterial cell wall.

An “immunogenic polypeptide” is meant to encompass antigenic or immunogenic polypeptides, e.g., poly-amino acid materials having epitopes or combinations of epitopes. As used herein, an immunogenic polypeptide is a polypeptide which, when introduced into a vertebrate, reacts with the immune system molecules of the vertebrate, i.e., is antigenic, and/or induces an immune response in the vertebrate, i.e., is immunogenic. It is likely that an immunogenic polypeptide will also be antigenic, but an antigenic polypeptide, because of its size or conformation, may not necessarily be immunogenic. Examples of antigenic and immunogenic polypeptides include, but are not limited to, polypeptides from infectious agents such as bacteria, viruses, parasites, or fungi, allergens such as those from pet dander, plants, dust, and other environmental sources, as well as certain self-polypeptides, for example, tumor-associated antigens.

Antigenic and immunogenic polypeptides of the invention can be used to prevent or treat, e.g., cure, ameliorate, lessen the severity of, or prevent or reduce contagion of viral, bacterial, fungal, and parasitic infectious diseases, as well as to treat allergies and proliferative diseases such as cancer.

In addition, antigenic and immunogenic polypeptides of the invention can be used to prevent or treat, e.g., cure, ameliorate, or lessen the severity of cancer including, but not limited to, cancers of oral cavity and pharynx (e.g., tongue, mouth, pharynx), digestive system (e.g., esophagus, stomach, small intestine, colon, rectum, anus, anal canal, anorectum, liver, gallbladder, pancreas), respiratory system (e.g., larynx, lung), bones, joints, soft tissues (including heart), skin, melanoma, breast, reproductive organs (e.g., cervix, endometirum, ovary, vulva, vagina, prostate, testis, penis), urinary system (e.g., urinary bladder, kidney, ureter, and other urinary organs), eye, brain, endocrine system (e.g., thyroid and other endocrine), lymphoma (e.g., hodgkin's disease, non-hodgkin's lymphoma), multiple myeloma, leukemia (e.g., acute lymphocytic leukemia, chronic lymphocytic leukemia, acute myeloid leukemia, chronic myeloid leukemia).

Antigenic and immunogenic polypeptides of the invention can be used to prevent or treat, e.g., cure, ameliorate, or lessen the severity of a disease caused by an infectious agent, e.g., viral, bacterial, fungal, and parasitic agents.

Examples of viral antigenic and immunogenic polypeptides include, but are not limited to, adenovirus polypeptides, alphavirus polypeptides, calicivirus polypeptides, e.g., a calicivirus capsid antigen, coronavirus polypeptides, distemper virus polypeptides, Ebola virus polypeptides, enterovirus polypeptides, flavivirus polypeptides, hepatitis virus (AE) polypeptides, e.g., a hepatitis B core or surface antigen, herpesvirus polypeptides, e.g., a herpes simplex virus or varicella zoster virus glycoprotein, immunodeficiency virus polypeptides, e.g., the human immunodeficiency virus envelope or protease, infectious peritonitis virus polypeptides, influenza virus polypeptides, e.g., an influenza A hemagglutinin, neuraminidase, or nucleoprotein, leukemia virus polypeptides, Marburg virus polypeptides, orthomyxovirus polypeptides, papilloma virus polypeptides, parainfluenza virus polypeptides, e.g., the hemagglutinin/neuraminidase, paramyxovirus polypeptides, parvovirus polypeptides, pestivirus polypeptides, picorna virus polypeptides, e.g., a poliovirus capsid polypeptide, pox virus polypeptides, e.g., a vaccinia virus polypeptide, rabies virus polypeptides, e.g., a rabies virus glycoprotein G, reovirus polypeptides, retrovirus polypeptides, and rotavirus polypeptides.

Examples of bacterial antigenic and immunogenic polypeptides include, but are not limited to, Actinomyces polypeptides, Bacillus polypeptides, e.g., immunogenic polypeptides from Bacillus anthracis, Bacteroides polypeptides, Bordetella polypeptides, Bartonella polypeptides, Borrelia polypeptides, e.g., B. burgdorferi OspA, Brucella polypeptides, Campylobacter polypeptides, Capnocytophaga polypeptides, Chlamydia polypeptides, Clostridium polypeptides, Corynebacterium polypeptides, Coxiella polypeptides, Dermatophilus polypeptides, Enterococcus polypeptides, Ehrlichia polypeptides, Escherichia polypeptides, Francisella polypeptides, Fusobacterium polypeptides, Haemobartonella polypeptides, Haemophilus polypeptides, e.g., H. influenzae type b outer membrane protein, Helicobacter polypeptides, Klebsiella polypeptides, L form bacteria polypeptides, Leptospira polypeptides, Listeria polypeptides, Mycobacteria polypeptides, Mycoplasma polypeptides, Neisseria polypeptides, Neorickettsia polypeptides, Nocardia polypeptides, Pasteurella polypeptides, Peptococcus polypeptides, Peptostreptococcus polypeptides, Pneumococcus polypeptides, Proteus polypeptides, Pseudomonas polypeptides, Rickettsia polypeptides, Rochalimaea polypeptides, Salmonella polypeptides, Shigella polypeptides, Staphylococcus polypeptides, Streptococcus polypeptides, e.g., S. pyogenes M proteins, Treponema polypeptides, and Yersinia polypeptides, e.g., Y. pestis F1 and V antigens.

Examples of parasitic antigenic and immunogenic polypeptides include, but are not limited to Balantidium coli polypeptides, Entamoeba histolytica polypeptides, Fasciola hepatica polypeptides, Giardia lamblia polypeptides, Leishmania polypeptides, and Plasmodium polypeptides (e.g., Plasmodium falciparum polypeptides).

Examples of fungal antigenic and immunogenic polypeptides include, but are not limited to, Aspergillus polypeptides, Candida polypeptides, Coccidiodes immitis or C. posadasii polypeptides, Cryptococcus polypeptides, Histoplasma polypeptides, Pneumocystis polypeptides, and Paracoccidiodes polypeptides.

Examples of tumor-associated antigenic and immunogenic polypeptides include, but are not limited to, tumor-specific immunoglobulin variable regions, GM2, Tn, sTn, Thompson-Friedenreich antigen (TF), Globo H, Le(y), MUC1, MUC2, MUC3, MUC4, MUC5AC, MUC5B, MUC7, carcinoembryonic antigens, beta chain of human chorionic gonadotropin (hCG beta). C35, HER2/neu, CD20, PSMA, EGFRvIII, KSA, PSA, PSCA, GP100, MAGE 1, MAGE 2, TRP 1, TRP 2, tyrosinase, MART-1, PAP, CEA, BAGE, MAGE, RAGE, and related proteins.

Compositions of the invention can further comprise other therapeutic agents. Examples of therapeutic agents include, but are not limited to, antimetabolites, alkylating agents, anthracyclines, antibiotics, and anti-mitotic agents. Antimetabolites include methotrexate, 6-mercaptopurine, 6-thioguanine, cytarabine, 5-fluorouracil decarbazine. Alkylating agents include mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin. Anthracyclines include daunorubicin (formerly daunomycin) and doxorubicin (also referred to herein as adriamycin). Additional examples include mitozantrone and bisantrene. Antibiotics include dactinomycin (formerly actinomycin), bleomycin, mithramycin, and anthramycin (AMC). Antimitotic agents include vincristine and vinblastine (which are commonly referred to as vinca alkaloids). Other cytotoxic agents include procarbazine, hydroxyurea, asparaginase, corticosteroids, mytotane (O,P′-(DDD)), interferons. Further examples of cytotoxic agents include, but are not limited to, ricin, doxorubicin, taxol, cytochalasin B, gramicidin D, ethidium bromide, etoposide, tenoposide, colchicin, dihydroxy anthracin dione, 1-dehydrotestosterone, and glucocorticoid. Analogs and homologs of such therapeutic agents are encompassed by the present invention.

### Bacterial Cell

The modified bacterium of the invention can be derived from a native form of the bacterial cell or can be a recombinant bacterial cell. In one embodiment, any bacterial cell described herein can also be unmodified and formulated with a separate ceramide-like glycolipid antigen and/or separate heterologous antigen. In another embodiment, a ceramide-like glycolipid of the invention is physically associated with a bacterial cell, e.g., incorporated into a bacterial cell wall, and used as an adjuvant to enhance an immune response, e.g., to an infectious agent or tumor.

Bacteria can be described as Gram-positive or Gram-negative. Beveridge T J, Biotech Histochem 76(3): 111-118 (2001); Gram H C, Fortschritte der Medizin 2: 185-189 (1884). Gram-positive bacteria are those that are stained dark blue or violet by Gram staining. Gram-positive bacteria are generally characterized by having as part of their cell wall structure peptidoglycan as well as polysaccharides and/or teichoic acids. The peptidoglycans, which are sometimes also called murein, are heteropolymers of glycan strands, which are cross-linked through short peptides. Gram-negative bacteria are generally surrounded by two membranes. The outer membrane contains lipopolysaccharides (LPS) and porins, and functions as a permeability barrier. Mycobacteria produce a thick mycolate-rich outer covering, which functions as an efficient barrier. Mycobacteria stain acid-fast and are phylogenetically related to the Gram-positive bacteria.

Bacterial or fungal agents that can cause disease or symptoms and that can be treated, prevented, and/or diagnosed by a modified bacterium, or composition, or vaccine composition of the present invention can include, but are not limited to the following Gram-negative and Gram-positive bacteria and bacterial families and fungi: Acinetobacter, Actinomycetes (e.g., Corynebacterium, Mycobacterium, Norcardia), Cryptococcus neoformans, Aspergillus, Bacillaceae (e.g., Bacillus anthracis), Bacteroidaceae, Blastomyces, Bordetella, Brucella, Candidia, Campylobacter, Clostridium, Coccidioides, Corynebacterium, Cryptococcus, Dermatophytes, Enterobacteriaceae (E. coli (e.g., Enterotoxigenic E. coli and Enterohemorrhagic E. coli) Klebsiella, Salmonella (e.g., Salmonella typhi, and Salmonella paratyphi), Serratia, Shigella, Yersinia, etc.), Erysipelothrix, Francisella, Helicobacter, Legionellaceae, Spirochaetaceae (e.g., Borrelia (e.g., Borrelia burgdorferi)), Leptospiraceae, Listeria, Mycoplasmatales, Mycobacterium leprae, Vibrionaceae (e.g., Vibrio cholerae), Neisseriaceae (e.g., Neisseria meningitidis, Neisseria gonorrhoeae), Actinobacillus, Haemophilus (e.g., Haemophilus influenza type B), Pasteurella, Pseudomonas, Rickettsiaceae, Chlamydiaceae, Treponema pallidum, Staphylococcaceae (e.g., Staphylococcus aureus, and Streptococcaceae (e.g., Streptococcus pneumoniae and Group B Streptococcus).

These bacterial or fungal families can cause the following diseases or symptoms, including, but not limited to: bacteremia, endocarditis, eye infections (conjunctivitis, tuberculosis, uveitis), gingivitis, opportunistic infections (e.g., AIDS related infections), paronychia, prosthesis-related infections, Reiter's Disease, respiratory tract infections, such as Whooping Cough or Empyema, sepsis, Lyme Disease, Cat-Scratch Disease, Dysentery, Paratyphoid Fever, food poisoning, Typhoid, pneumonia, Gonorrhea, meningitis (e.g., mengitis types A and B), Chlamydia, Syphilis, Diphtheria, Leprosy, Paratuberculosis, Tuberculosis (TB), Hansen's disease, Pulmonary disease resembling tuberculosis, Lymphadenitis, Skin disease, or Disseminated disease, Lupus, Botulism, gangrene, tetanus, impetigo, Rheumatic Fever, Scarlet Fever, sexually transmitted diseases, skin diseases (e.g., cellulitis, dermatocycoses), toxemia, urinary tract infections, and wound infections.

A modified bacterium, composition, or vaccine composition of the invention can be used to treat, prevent, and/or diagnose any of these symptoms or diseases. In specific embodiments, compositions of the invention are used to treat: tuberculosis, pulmonary disease resembling tuberculosis, lymphadenitis, skin disease, disseminated disease, bubonic plague, pneumonic plague, tularemia, Legionairre's disease, anthrax, typhoid fever, paratyphoid fever, foodborne illness, listeriosis, malaria, HIV, SIV, HPV, influenza, hepatitis (HAV, HBV, and HCV), and cancer.

### Mycobacteria

The genus Mycobacterium includes pathogens known to cause serious diseases in mammals, including, for example, tuberculosis and leprosy. Mycobacterium (also referred to as mycobacteria) do not contain endospores or capsules, and are usually considered Gram-positive. In addition to the usual fatty acids found in membrane lipids, mycobacteria have a wide variety of very long-chain saturated (C₁₈-C₃₂) and monounsaturated (up to C₂₆) n-fatty acids. The occurrence of α-alkyl β-hydroxy very long-chain fatty acids, i.e., mycolic acids, is a hallmark of mycobacteria and related species. Mycobacterial mycolic acids are large (C₇₀-C₉₀) with a large α-branch (C₂₀-C₂₅). The main chain contains one or two double bonds, cyclopropane rings, epoxy groups, methoxy groups, keto groups or methyl branches. Such acids are major components of the cell wall, occurring mostly esterified in clusters of four on the terminal hexa-arabinofuranosyl units of the major cell-wall polysaccharides called arabinogalactans. They are also found esterified to the 6 and 6′ positions of trehalose to form ‘cord factor’. Small amounts of mycolate are also found esterified to glycerol or sugars such as trehalose, glucose and fructose depending on the sugars present in the culture medium. Mycobacteria also contain a wide variety of methyl-branched fatty acids. These include 10-methyl C₁₈ fatty acid (tuberculostearic acid found esterified in phosphatidyl inositide mannosides), 2,4-dimethyl C₁₄ acid and mono-, di- and trimethyl-branched C₁₄ to C₂₅ fatty acids found in trehalose-containing lipooligosaccharides, trimethyl unsaturated C₂₇ acid (phthienoic acid), tetra-methyl-branched C₂₈-C₃₂ faccy acids (mycocerosic acids) and shorter homologues found in phenolic glycolipids and phthiocerol esters, and multiple methyl-branched phthio-ceranic acids such as hepamethyl-branched C₃₇ acid and oxygenated multiple methyl-branched acids such as 17-hydroxy-2,4,6,8,10,12,14,16-octamethyl C₄₀ acid found in sulpholipids. In addition, mycocerosic acids and other branched acids are esterified to phthicerol and phenolphthicerol and their derivivates. Kolattukudy et al., Mol. Microbio. 24(2):263-270 (1997). Evidence implicates specific cell envelope lipids in Mtb pathogenesis. Rao, et al., J. Exp. Med., 201(4):535-543 (2005).

Mycobacterium species include, but are not limited to: M. abscessus; M. africanum; M. agri; M. aichiense; M. alvei; M. arupense; M. asiaticum; M. aubagnense; M. aurum; M. austroafricanum; Mycobacterium avium complex (MAC); M. avium; M. avium paratuberculosis, which has been implicated in Crohn's disease in humans and Johne's disease in sheep; M. avium silvaticum; M. avium “hominissuis”; M. colombiense; M. boenickei; M. bohemicum; M. bolletii; M. botniense; M. bovis; M. branderi; M. brisbanense; M. brumae M. canariasense; M. caprae; M. celatum; M. chelonae; M. chimaera; M. chitae; M. chlorophenolicum; M. chubuense; M. conceptionense; M. confluentis; M. conspicuum; M. cookii; M. cosmeticum; M. diernhoferi; M. doricum; M. duvalii; M. elephantis; M. fallax; M. farcinogenes; M. flavescens; M. florentinum; M. fluoroanthenivorans; M. fortuitum; M. fortuitum subsp. acetamidolyticum; M. frederiksbergense; M. gadium; M. gastri; M. genavense; M. gilvum; M. goodii; M. gordonae; M. haemophilum; M. hassiacum; M. heckeshornense; M. heidelbergense; M. hiberniae; M. hodleri; M. holsaticum; M. houstonense; M. immunogenum; M. interjectum; M. intermedium; M. intracellulare; M. kansasii; M. komossense; M. kubicae; M. kumamotonense; M. lacus; M. lentiflavum; M. leprae, which causes leprosy; M. lepraemurium; M. madagascariense; M. mageritense; M. malmoense; M. marinum; M. massiliense; M. microti; M. monacense; M. montefiorense; M. moriokaense; M. mucogenicum; M. murale; M. nebraskense; M. neoaurum; M. neworleansense; M. nonchromogenicum; M. novocastrense; M. obuense; M. palustre; M. parafortuitum; M. parascrofulaceum; M. parmense; M. peregrinum; M. phlei; M. phocaicum; M. pinnipedii; M. porcinum; M. poriferae; M. pseudoshottsii; M. pulveris; M. psychrotolerans; M. pyrenivorans; M. rhodesiae; M. saskatchewanense; M. scrofulaceum; M. senegalense; M. seoulense; M. septicum; M. shimoidei; M. shottsii; M. simiae; M. smegmatis; M. sphagni; M. szulgai; M. terrae; M. thermoresistibile; M. tokaiense; M. triplex; M. triviale; Mycobacterium tuberculosis complex (MTBC), members are causative agents of human and animal tuberculosis (M. tuberculosis, the major cause of human tuberculosis; M. bovis; M. bovis BCG; M. africanum; M. canetti; M. caprae; M. pinnipedii'); M. tusciae; M. ulcerans, which causes the “Buruli”, “Bairnsdale, ulcer”; M. vaccae; M. vanbaalenii; M. wolinskyi; and M. xenopi.

Mycobacteria can be classified into several groups for purpose of diagnosis and treatment, for example: M. tuberculosis complex (MTB) which can cause tuberculosis: M. tuberculosis, M. africanum, M. bovis, M. bovis BCG, M. caprae, M. microti, M. pinnipedii, the dassie bacillus, and M. canettii (proposed name) (Somoskovi, et al., J. Clinical Microbio 45(2):595-599 (2007)); M. leprae which causes Hansen's disease or leprosy; nontuberculous mycobacteria (NTM) are all the other mycobacteria which can cause pulmonary disease resembling tuberculosis, lymphadenitis, skin disease, or disseminated disease. MTB members show a high degree of genetic homogeneity. Somoskovi (2007). The mycobacteria of the invention can include recombinant mycobacteria. For example, recombinant mycobacterial cells, e.g., recombinant BCG cells, e.g., rBCG30 cells.

### Recombinant Bacteria

A modified bacterium of the invention can also include a recombinant bacterial cell, e.g., a recombinant mycobacterial cell. A non-limiting example of a recombinant bacterial cell is rBCG30, which is derived from a vaccine strain of BCG and has been genetically modified to overexpress the immunodominant antigen Ag85B. See Doherty and Anderson, Clinical Microbio Reviews 18(4): 687-702 (2005). Other examples of recombinant bacterial cells suitable for producing glycolipid modified bacterium of the invention include, but are not limited to BCG-HIV; BCG-SIV; BCG-HCV; rBCG/IL-2, and recombinant M. smegmatis expressing HIV peptides (See e.g., Adlovini and Young, Nature 479-482 (1994); Yasutomi et al., J. of Immunol. 150(7):3101-3107 (1993); Uno-Furuta et al, Vaccine 21(23): 3149-3156 (2003); Matsumoto et al., J. Exp. Med. 188(5): 845-854 (1998); Yamada et al., J. of Urology 164(2): 526-531 (2000); Cayabyab et al., J. of Virology 80(4): 1645-1652 (2006); Stover et al., Nature 351: 456-460 (1991); and Bloom et al., U.S. Pat. No. 5,504,005).

In one embodiment, the modified bacterium comprises a recombinant bacterial cell engineered to express a heterologous antigen, e.g., a polypeptide encoded by non-native polynucleotides, e.g., BCG/SIV-Gag or BCG-HIV, wherein the recombinant bacterial cell is physically associated with a ceramide-like glycolipid. The invention further relates to a composition or vaccine composition comprising a modified bacterium of the invention, wherein the bacterial cell is native or recombinant. In certain embodiments, the recombinant modified bacteria are antigen carriers for delivery of heterologous antigens against infectious agents or tumors. In further embodiments, the recombinant modified bacteria is used to enhance priming of an immune response against the heterologous antigen.

The invention further relates to a recombinant (genetically engineered) modified bacterium, e.g., a ceramide-like glycolipid/mycobacterial complex, which expresses DNA encoding a heterologous polypeptide. The DNA can be incorporated into the bacterial genome or exist extrachromosomally using standard genetic engineering techniques. Recombinant bacteria of the invention can be engineered using vectors for the introduction of DNA of interest, e.g., DNA encoding heterologous antigens or immunogens, into bacteria, e.g., mycobacteria.

As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Certain vectors are capable of autonomous replication in a host cell into which they are introduced (e.g., bacterial vectors having a bacterial origin of replication). The vectors of the present invention are capable of directing the expression of genes encoding polypeptides, e.g., immunogenic polypeptides, to which they are operatively linked. Such vectors are referred to herein as “expression vectors”. In general, expression vectors of utility in recombinant DNA techniques are often in the form of plasmids.

Expression vectors comprising nucleic acids encoding polypeptides can be useful in the present invention, e.g., for expression of immunogenic polypeptides, from recombinant bacteria, e.g., glycolipid modified recombinant mycobacteria. The choice of vector and expression control sequences to which such nucleic acids are operably linked depends on the functional properties desired, e.g., protein expression, and the host cell to be transformed.

Expression control elements useful for regulating the expression of an operably linked coding sequence are known in the art. Examples include, but are not limited to, inducible promoters, constitutive promoters, secretion signals, and other regulatory elements. When an inducible promoter is used, it can be controlled, e.g., by a change in nutrient status of host cell medium or a change in temperature. Polynucleotide and nucleic acid coding regions of the present invention can be associated with additional coding regions which encode secretory or signal peptides, which direct the secretion of a polypeptide encoded by a polynucleotide of the present invention.

In one embodiment, bacterial expression of a polynucleotide of interest occurs extrachromosomally, e.g., from a plasmid (e.g., episomally). For example, a gene of interest is cloned into a plasmid and introduced into a cultured mycobacterial cell, e.g., BCG or M. smegmatis, where the gene of interest encodes a polypeptide of interest, e.g., an immunogenic polypeptide. Plasmid vectors containing replicon and control sequences which are derived from species compatible with the host cell, e.g., mycobacterial host cells, are used. The vector can carry a replication site, as well as marking sequences which are capable of providing phenotypic selection in transformed cells.

A vector of the invention can include, but is not limited to a prokaryotic replicon, i.e., a DNA sequence having the ability to direct autonomous replication and maintenance of the recombinant DNA molecule extra-chromosomally in a bacterial host cell. Such replicons are well known in the art. In addition, vectors that include a prokaryotic replicon may also include a gene whose expression confers a detectable marker such as a drug resistance. Non-limiting examples of bacterial drug-resistance genes are those that confer resistance to ampicillin or tetracycline.

Vectors that include a prokaryotic replicon can also include a prokaryotic or bacteriophage promoter for directing expression of the coding gene sequences in a bacterial host cell. Promoter sequences compatible with bacterial hosts typically are provided in plasmid vectors containing convenient restriction sites for insertion of a DNA segment to be expressed. Examples of promoters which can be used for expression in prokaryotic host cells, e.g., mycobacterial host cells, include, but are not limited to heat shock promoters, stress protein promoters, pMTB30 promoters, B-lactamase (penicillinase) promoters, lactose promoters, promoters expressing kanamycin resistance, promoters expressing chloramphenicol resistance, and cI promoters (see also Sambrook et al.). Various prokaryotic cloning vectors can be used in the invention. Examples of such plasmid vectors include, but are not limited to pUC8, pUC9, pBR322 and pBR329 (BioRad® Laboratories), pPL, pEMBL and pKK223 (Pharmacia) (see also Sambrook et al.).

Vector DNA can be introduced into prokaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook et al. (MOLECULAR CLONING: A LABORATORY MANUAL 2nd ed., Cold Spring Harbor Laboratory, Cold Spring Harbor Laboratory Press, Cold Spring Harbor, N.Y., 1989), and other laboratory manuals. Transformation of host cells, e.g., bacterial cells such as mycobacterial cells or glycolipid modified mycobacterial cells, can be accomplished by conventional methods suited to the vector and host cell employed. For transformation of prokaryotic host cells, e.g., mycobacterial cells, electroporation and salt treatment methods can be employed (Cohen et al., Proc. Natl. Acad. Sci. USA 69:2110-14 (1972)), as well as other techniques known in the art.

As used herein, the term “polypeptide” is intended to encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids, and does not refer to a specific length of the product. Thus, peptides, dipeptides, tripeptides, oligopeptides, “protein,” “amino acid chain,” or any other term used to refer to a chain or chains of two or more amino acids, are included within the definition of “polypeptide,” and the term “polypeptide” can be used instead of, or interchangeably with any of these terms. The term “polypeptide” is also intended to refer to the products of post-expression modifications of the polypeptide. A polypeptide can be derived from a natural biological source or produced by recombinant technology, but is not necessarily translated from a designated nucleic acid sequence. It can be generated in any manner, including by chemical synthesis.

A polypeptide of the invention can be of a size of about 3 or more, 5 or more, 10 or more, 20 or more, 25 or more, 50 or more, 75 or more, 100 or more, 200 or more, 500 or more, 1,000 or more, or 2,000 or more amino acids.

By an “isolated polypeptide” or a fragment, variant, or derivative thereof is intended a polypeptide that is not in its natural milieu. No particular level of purification is required. For example, an isolated polypeptide can be removed from its native or natural environment. Recombinantly produced polypeptides and proteins expressed in host cells or as a component of a recombinant bacterial vaccine are considered isolated for purposed of the invention, as are native or recombinant polypeptides which have been separated, fractionated, or partially or substantially purified by any suitable technique.

Also included as polypeptides of the present invention are fragments, derivatives, analogs, or variants of the foregoing polypeptides, and any combination thereof. The terms “fragment.” “variant,” “derivative” and “analog” when referring to polypeptides of the present invention include any polypeptides that retain at least some of the biological, antigenic, or immunogenic properties of the corresponding native polypeptide.

The term “polynucleotide” is intended to encompass a singular nucleic acid as well as plural nucleic acids, and refers to an isolated nucleic acid molecule or construct, e.g., messenger RNA (mRNA), virally-derived RNA, or plasmid DNA (pDNA). A polynucleotide can comprise a conventional phosphodiester bond or a non-conventional bond (e.g., an amide bond, such as found in peptide nucleic acids (PNA)). The term “nucleic acid” refers to any one or more nucleic acid segments, e.g., DNA or RNA fragments, present in a polynucleotide. RNA of the present invention can be single stranded or double stranded.

By “isolated” nucleic acid or polynucleotide is intended a nucleic acid molecule, DNA or RNA, which has been removed from its native environment. For example, a recombinant polynucleotide encoding a therapeutic polypeptide contained in a vector is considered isolated for the purposes of the present invention. Further examples of an isolated polynucleotide include recombinant polynucleotides maintained in heterologous host cells, e.g., recombinant bacterial cells, or purified (partially or substantially) polynucleotides in solution. Isolated RNA molecules include in vivo or in vitro RNA transcripts of the present invention, as well as positive and negative strand forms, and double-stranded forms, of pestivirus vectors disclosed herein. Isolated polynucleotides or nucleic acids according to the present invention further include such molecules produced synthetically. In addition, a polynucleotide or a nucleic acid can be or can include a regulatory element such as a promoter, ribosome binding site, or a transcription terminator.

As used herein, a “heterologous polynucleotide” or a “heterologous nucleic acid” or a “heterologous gene” or a “heterologous sequence” or an “exogenous DNA segment” refers to a polynucleotide, nucleic acid or DNA segment that originates from a source foreign to the particular host cell, or, if from the same source, is modified from its original form. A heterologous gene in a host cell includes a gene that is endogenous to the particular host cell, but has been modified. Thus, the terms refer to a DNA segment which is foreign or heterologous to the cell, or homologous to the cell but in a position within the host cell nucleic acid in which the element is not ordinarily found.

As used herein, a “coding region” is a portion of nucleic acid which consists of codons translated into amino acids. Although a “stop codon” (TAG, TGA, or TAA) is not translated into an amino acid, it can be considered to be part of a coding region, if present, but any flanking sequences; for example promoters, ribosome binding sites, transcriptional terminators, introns, 5′ and 3′ non-translated regions, and the like, are not part of a coding region. Two or more coding regions of the present invention can be present in a single polynucleotide construct, e.g., on a single vector, or in separate polynucleotide constructs, e.g., on separate (different) vectors. Furthermore, any vector can contain a single coding region, or can comprise two or more coding regions. In addition, a vector, polynucleotide, or nucleic acid of the invention can encode two or more heterologous coding regions, either fused or unfused. Heterologous coding regions include without limitation specialized elements or motifs, such as a secretory signal peptide or a heterologous functional domain.

In certain embodiments, the polynucleotide or nucleic acid is DNA. In the case of DNA, a polynucleotide comprising a nucleic acid, which encodes a polypeptide normally can include a promoter and/or other transcription or translation control elements operably associated with one or more coding regions. An operable association is when a coding region for a gene product, e.g., a polypeptide, is associated with one or more regulatory sequences in such a way as to place expression of the gene product under the influence or control of the regulatory sequence(s). Two DNA fragments (such as a polypeptide coding region and a promoter associated therewith) are “operably associated” if induction of promoter function results in the transcription of mRNA encoding the desired gene product and if the nature of the linkage between the two DNA fragments does not interfere with the ability of the expression regulatory sequences to direct the expression of the gene product or interfere with the ability of the DNA template to be transcribed. Thus, a promoter region would be operably associated with a nucleic acid encoding a polypeptide if the promoter was capable of effecting transcription of that nucleic acid. The promoter can be a cell-specific promoter that directs substantial transcription of the DNA only in predetermined cells. Other transcription control elements, besides a promoter, for example enhancers, operators, repressors, and transcription termination signals, can be operably associated with the polynucleotide to direct cell-specific transcription.

By “a reference amino acid sequence” is meant the specified sequence without the introduction of any amino acid substitutions. As one of ordinary skill in the art would understand, if there are no substitutions, the “isolated polypeptide” of the invention comprises an amino acid sequence which is identical to the reference amino acid sequence.

As used herein, a “heterologous antigen” or a “heterologous polypeptide” refers to an antigen or polypeptide that originates from a source foreign to the particular cell, or, if from the same source, is modified from its original form. In certain embodiments, a “heterologous antigen” or a “heterologous polypeptide” is expressed by a host cell, e.g., a modified recombinant bacterial cell. A heterologous antigen or polypeptide expressed by a host cell includes an antigen or polypeptide that is endogenous to the particular host cell, but has been modified.

Polypeptides described herein can have various alterations such as substitutions, insertions or deletions. Exemplary amino acids that can be substituted in the polypeptide include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

Corresponding fragments of polypeptides at least 70%, 75%, 80%, 85%, 90%, or 95% identical to the polypeptides and reference polypeptides described herein are also contemplated.

As known in the art, “sequence identity” between two polypeptides is determined by comparing the amino acid sequence of one polypeptide to the sequence of a second polypeptide. When discussed herein, whether any particular polypeptide is at least about 70%, 75%, 80%, 85%, 90% or 95% identical to another polypeptide can be determined using methods and computer programs/software known in the art such as, but not limited to, the BESTFIT program (Wisconsin Sequence Analysis Package, Version 8 for Unix, Genetics Computer Group, University Research Park, 575 Science Drive, Madison, Wis. 53711). BESTFIT uses the local homology algorithm of Smith and Waterman, Advances in Applied Mathematics 2:482-489 (1981), to find the best segment of homology between two sequences. When using BESTFIT or any other sequence alignment program to determine whether a particular sequence is, for example, 95% identical to a reference sequence according to the present invention, the parameters are set, of course, such that the percentage of identity is calculated over the full length of the reference polypeptide sequence and that gaps in homology of up to 5% of the total number of amino acids in the reference sequence are allowed.

### Ceramide-Like Glycolipid Antigens

Ceramide-like glycolipid antigens useful within the present invention include without limitation those ceramide-like glycolipids which are capable of modulating an immune response in an animal when presented with a bacterial cell, e.g., by incorporation of the ceramide-like glycolipid into the cell wall of a bacterial cell. The antigens may be derived from foreign antigens or from autoantigens. Further, the ceramide-like glycolipid antigens can be synthetic. Suitable antigens are disclosed, e.g., in Porcelli, U.S. Patent Appl. Publ. No. 2006/0052316, Tsuji, U.S. Patent Appl. Publ. No. 2006/0211856, Jiang, U.S. Patent Appl. Publ. No. 2006/0116331, Hirokazu et al., U.S. Patent Appl. Publ. No. 2006/0074235, Tsuji et al., U.S. Patent Appl. Publ. No. 2005/0192248, Tsuji, U.S. Patent Application No. 2004/0127429, and Tsuji et al., U.S. Patent Application No. 2003/0157135, which are incorporated herein by reference. In certain embodiments, the ceramide-like glycolipid is α-GalCer or an analog thereof. In other embodiments, the ceramide-like glycolipid is a α-C-GalCer or an analog thereof.

The term “optionally substituted” as used herein means either unsubstituted or substituted with one or more substituents including halogen (F, Cl, Br, I), alkyl, substituted alkyl, aryl, substituted aryl, or alkoxy.

The term “alkyl”, as used herein by itself or part of another group refers to a straight-chain or branched saturated aliphatic hydrocarbon typically having from one to eighteen carbons or the number of carbons designated. In one such embodiment, the alkyl is methyl. Non-limiting exemplary alkyl groups include ethyl, n-propyl, isopropyl, and the like.

The term “substituted alkyl” as used herein refers to an alkyl as defined above having one or more halogen (F, Cl, Br, I) substitutes.

The term “heterocycle” as used herein means a 3- to 10-membered monocyclic or bicyclic heterocyclic ring which is either saturated, unsaturated non-aromatic, or aromatic containing up to 4 heteroatoms. Each heteroatom is independently selected from nitrogen, which can be quaternized; oxygen; and sulfur, including sulfoxide and sulfone. The heterocycle can be attached via a nitrogen, sulfur, or carbon atom. Representative heterocycles include pyridyl, furyl, thiophenyl, pyrrolyl, oxazolyl, imidazolyl, thiazolyl, thiadiazolyl, isoxazolyl, pyrazolyl, isothiazolyl, pyridazinyl, pyrimidinyl, pyrazinyl, triazinyl, morpholinyl, pyrrolidinonyl, pyrrolidinyl, piperidinyl, piperazinyl, hydantoinyl, valerolactamyl, oxiranyl, oxetanyl, tetrahydrofuranyl, tetrahydropyranyl, tetrahydropyrindinyl, tetrahydropyrimidinyl, tetrahydrothiophenyl, tetrahydrothiopyranyl, quinolinyl, -isoquinolinyl, -chromonyl, -coumarinyl, -indolyl, -indolizinyl, -benzo[b]furanyl, -benzo[b]thiophenyl, -indazolyl, -purinyl, -4H-quinolizinyl, -isoquinolyl, -quinolyl, -phthalazinyl, -naphthyridinyl, -carbazolyl, and the like. The term heterocycle also includes heteroaryls.

The term “aryl” as used herein by itself or part of another group refers to monocyclic and bicyclic aromatic ring systems typically having from six to fourteen carbon atoms (i.e., C₆-C₁₄ aryl) such as phenyl, 1-naphthyl, and the like.

The term “substituted aryl” as used herein refers to an aryl as defined above having one or more substitutes including halogen (F, Cl, Br, I) or alkoxy.

The term “aralkyl” as used herein by itself or part of another group refers to an alkyl as defined above having one or more aryl substituents. Non-limiting exemplary aralkyl groups include benzyl, phenylethyl, diphenylmethyl, and the like.

The term “alkoxy” as used herein by itself or part of another group refers to an alkyl attached to a terminal oxygen atom. Non-limiting exemplary alkoxy groups include methoxy, ethoxy and the like.

The term “alkane” as used herein means a straight chain or branched non-cyclic saturated hydrocarbon. Representative straight chain alkane include -methyl, -ethyl, -n-propyl, -n-butyl, -n-pentyl, -n-hexyl, -n-heptyl, -n-octyl, -n-nonyl and -n-decyl. Representative branched alkane include -isopropyl, -sec-butyl, -isobutyl, -tert-butyl, -isopentyl, -neopentyl, 1-methylbutyl, 2-methylbutyl, 3-methylbutyl, 1,1-dimethylpropyl, 1,2-dimethylpropyl, 1-methylpentyl, 2-methylpentyl, 3-methylpentyl, 4-methylpentyl, 1-ethylbutyl, 2-ethylbutyl, 3-ethylbutyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-methylhexyl, 2-methylhexyl, 3-methylhexyl, 4-methylhexyl, 5-methylhexyl, 1,2-dimethylpentyl, 1,3-dimethylpentyl, 1,2-dimethylhexyl, 1,3-dimethylhexyl, 3,3-dimethylhexyl, 1,2-dimethylheptyl, 1,3-dimethylheptyl, and 3,3-dimethylheptyl.

The term “alkene” as used herein means a straight chain or branched non-cyclic hydrocarbon having at least one carbon-carbon double bond. Representative straight chain and branched alkene include -vinyl, -allyl, -1-butenyl, -2-butenyl, -isobutylenyl, -1-pentenyl, -2-pentenyl, -3-methyl-1-butenyl, -2-methyl-2-butenyl, -2,3-dimethyl-2-butenyl, -1-hexenyl, -2-hexenyl, -3-hexenyl, -1-heptenyl, -2-heptenyl, -3-heptenyl, -1-octenyl, -2-octenyl, -3-octenyl, -1-nonenyl, -2-nonenyl, -3-nonenyl, -1-decenyl, -2-decenyl, -3-decenyl and the like.

The term “cylcoalkane” as used herein means a saturated cyclic hydrocarbon having from 3 to 15 carbon atoms. Representative cycloalkanes are cyclopropyl, cyclopentyl and the like.

The term “alkylcycloalkene” as used herein by itself or part of another group refers to an alkyl as defined above attached a cylcoalkane as defined above.

The term “cylcoalkene” as used herein means means a mono-cyclic non-aromatic hydrocarbon having at least one carbon-carbon double bond in the cyclic system and from 5 to 15 carbon atoms. Representative cycloalkenes include -cyclopentenyl, -cyclopentadienyl, -cyclohexenyl, -cyclohexadienyl, -cycloheptenyl, -cycloheptadienyl, -cycloheptatrienyl, -cyclooctenyl, -cyclooctadienyl, cyclooctatrienyl, -cyclooctatetraenyl, -cyclononenyl-cyclononadienyl, -cyclodecenyl, -cyclodecadienyl and the like. The term “cycloalkene” also include bicycloalkenes and tricycloalkenes. The term “bicycloalkene” as used herein means a bicyclic hydrocarbon ring system having at least one carbon-carbon double bond in one of the rings and from 8 to 15 carbon atoms. Representative bicycloalkenes include, but are not limited to, -indenyl, -pentalenyl, -naphthalenyl, -azulenyl, -heptalenyl, -1,2,7,8-tetrahydronaphthalenyl, and the like. The term “tricycloalkene” as used herein, means a tri-cyclic hydrocarbon ring system having at least one carbon-carbon double bond in one of the rings and from 8 to 15 carbon atoms. Representative tricycloalkenes include, but are not limited to, -anthracenyl.-phenanthrenyl, -phenalenyl, and the like.

The term “aromatic ring” as used herein means a 5 to 14 membered aromatic carbocyclic ring, including both mono, bicyclic, and tricyclic ring systems. Representative aromatic rings are phenyl, napthyl, anthryl and phenanthryl.

The phrase “oxo” as used herein, means a double bond to oxygen. i.e., C═O.

The term “monosaccharide” as used herein means any of the simple sugars that serve as building blocks for carbohydrates. Examples of monosaccharides include glucose, fucose, galactose, and mannose.

The term “hydroxyl” as used herein refers to a functional group composed of one oxygen bonded to one hydrogen, with the oxygen covalently bonded to another atom, e.g., a carbon. A “hydroxyl-protected glycolipid” of the invention includes a glycolipid, e.g., a synthetic glycolipid, having a hydroxyl group coupled to a protection group.

Other ceramide-like glycolipids for use in the present invention include, but are not limited to the ceramide-like glycolipid antigens in Table 1.

| | CHO | |
|---|---|---|
| Compound | group | Structure |
| DB04-1 (KRN7000) | α-D-Gal | 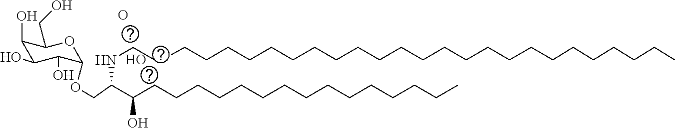 |
| DB01-1 | α-D-Gal | 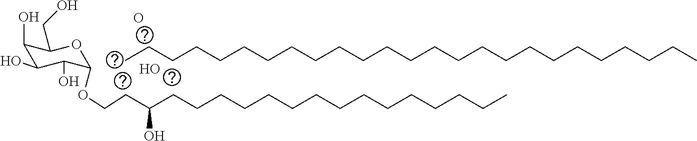 |
| DB02-1 | α-D-Glu | 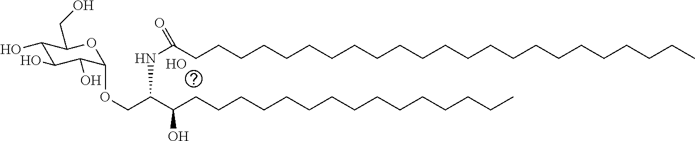 |
| DB02-2 | α-D-Man | 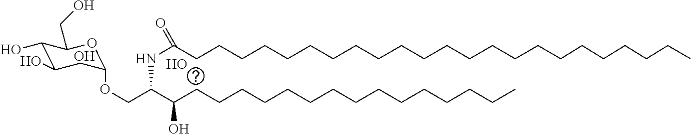 |
| DB03-2 | α-D-Gal | 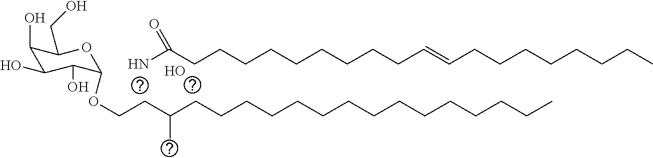 |
| DB03-3 | α-D-Gal | 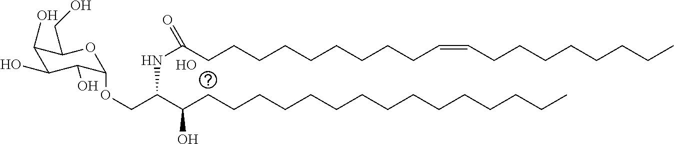 |
| DB03-4 | α-D-Gal | 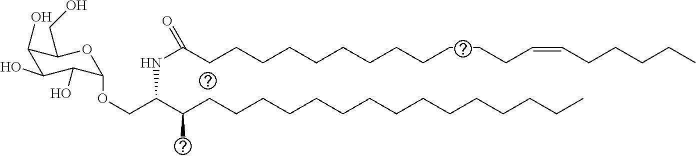 [SMILES: CCCCC/C=C\CCCCCCCCCCCCC(=O)N[C@@H](COC1OC(CO)C(O)C(O)C1O)[C@H](C)CCCCCCCCCCCCCCC] |
| DB03-5 | α-D-Gal | 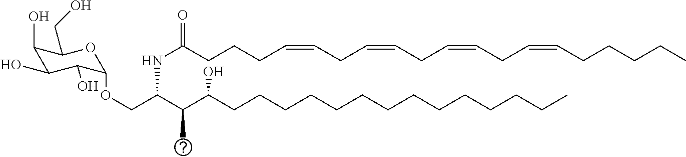 [SMILES: CCCCC/C=C\C/C=C\C/C=C\C/C=C\CCCC(=O)N[C@@H](COC1OC(CO)C(O)C(O)C1O)[C@H](C)[C@H](O)CCCCCCCCCCCCCC] |
| DB03-6 | α-D-Gal | 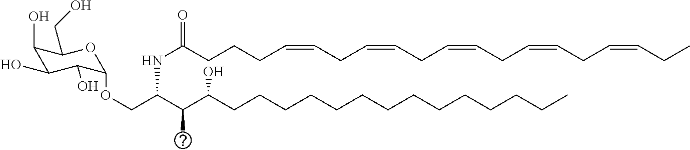 [SMILES: CC/C=C\C/C=C\C/C=C\C/C=C\C/C=C\CCCC(=O)N[C@@H](COC1OC(CO)C(O)C(O)C1O)[C@H](C)[C@H](O)CCCCCCCCCCCCCC] |
| DB04-11 | α-D-Gal | 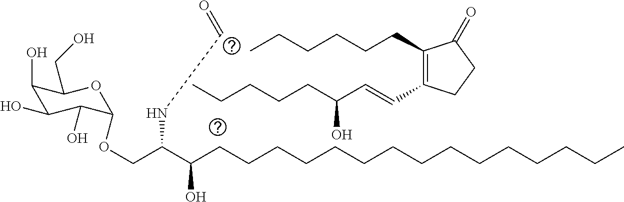 |
| DB06-9 | D-Gal ( [symbol] 1 → 2)D- Gal | 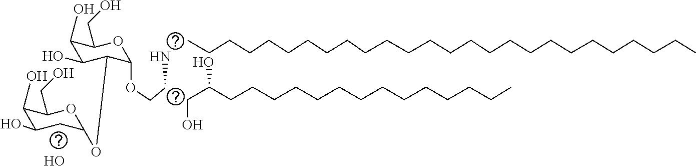 |
| DB08-1 | α-D-Gal | 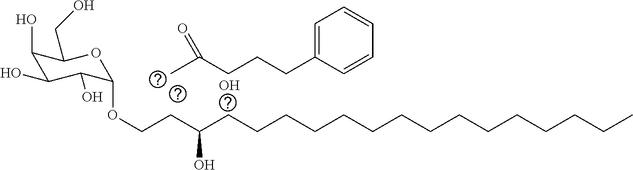 |
| DB08-2 | α-D-Gal | 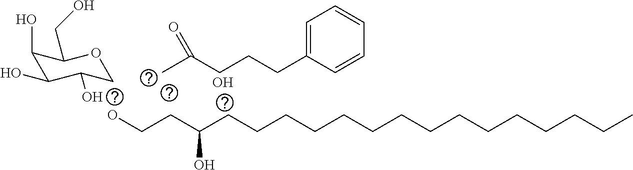 |
| DB08-3 | α-D-Gal | 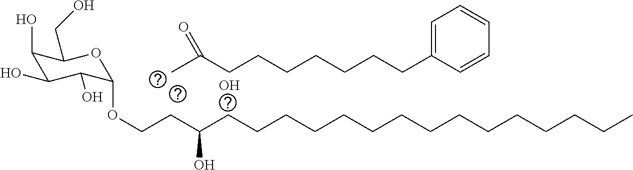 |
| DB09-1 | α-D-Gal | 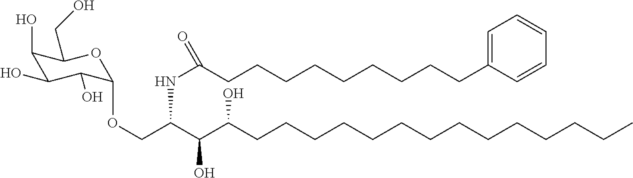 [SMILES: CCCCCCCCCCCCCC[C@@H](O)[C@@H](O)[C@H](COC1OC(CO)C(O)C(O)C1O)NC(=O)CCCCCCCCCc1ccccc1] |
| DB09-2 | α-D-Gal | 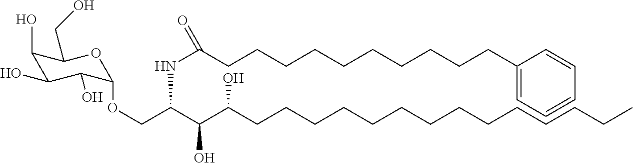 [SMILES: CCCCCCCCCCCCCC[C@@H](O)[C@@H](O)[C@H](COC1OC(CO)C(O)C(O)C1O)NC(=O)CCCCCCCCCCc1ccccc1] |
| AH04-1 (OCH) | α-D-Gal | 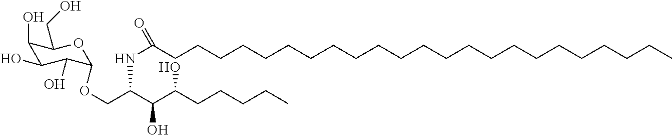 [SMILES: CCCCCCCCCCCCCCCCCCCCCCCC(=O)N[C@@H](COC1OC(CO)C(O)C(O)C1O)[C@H](O)[C@H](O)CCCCC] |
| YTC03-00 | α-D-Gal | 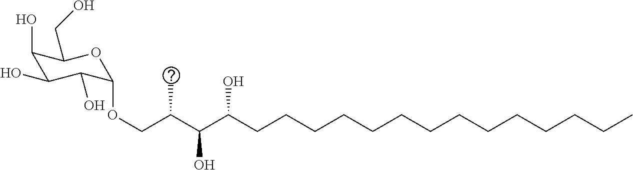 [SMILES: CCCCCCCCCCCCCC[C@@H](O)[C@@H](O)[C@@H](C)COC1OC(CO)C(O)C(O)C1O] |
| YTC03-4 | α-D-Gal | 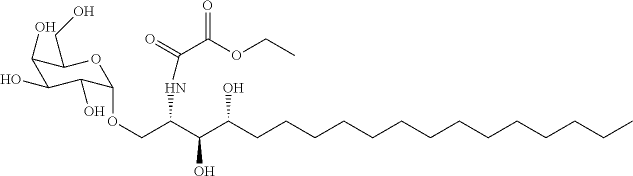 [SMILES: CCCCCCCCCCCCCC[C@@H](O)[C@@H](O)[C@H](COC1OC(CO)C(O)C(O)C1O)NC(=O)C(=O)OCC] |
| YTC03-6 | α-D-Gal | 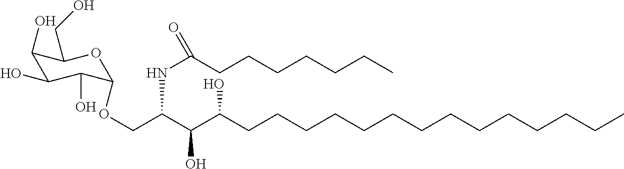 [SMILES: CCCCCCCCCCCCCC[C@@H](O)[C@@H](O)[C@H](COC1OC(CO)C(O)C(O)C1O)NC(=O)CCCCCCC] |
| YTC03-7 | α-D-Gal | 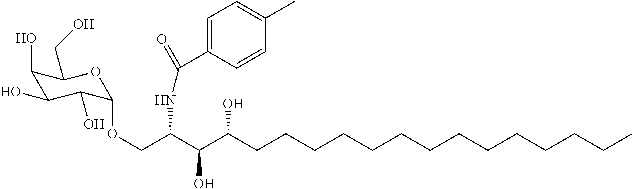 [SMILES: CCCCCCCCCCCCCC[C@@H](O)[C@@H](O)[C@H](COC1OC(CO)C(O)C(O)C1O)NC(=O)c1ccc(C)cc1] |
| YTC03-15 | α-D-Gal | 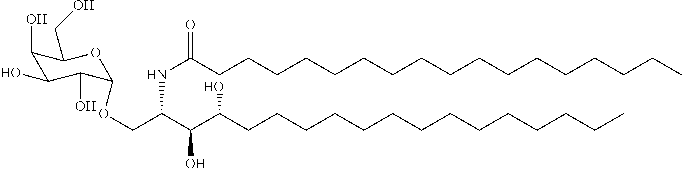 [SMILES: CCCCCCCCCCCCCCCCCC(=O)N[C@@H](COC1OC(CO)C(O)C(O)C1O)[C@H](O)[C@H](O)CCCCCCCCCCCCCC] |
| YTC03-16 | α-D-Gal | 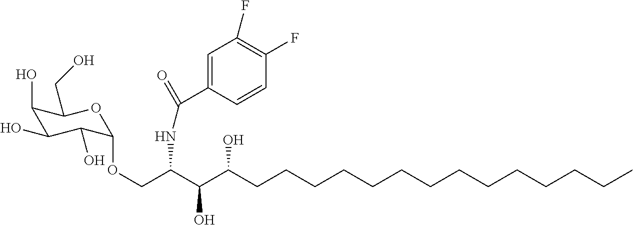 [SMILES: CCCCCCCCCCCCCC[C@@H](O)[C@@H](O)[C@H](COC1OC(CO)C(O)C(O)C1O)NC(=O)c1ccc(F)c(F)c1] |
| YTC03-17 | α-D-Gal | 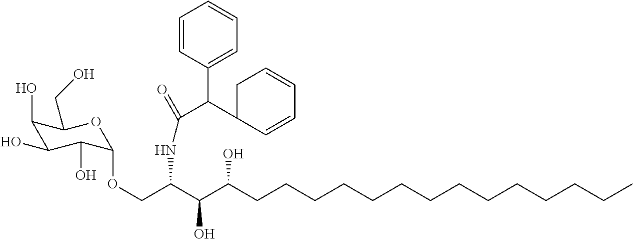 [SMILES: CCCCCCCCCCCCCC[C@@H](O)[C@@H](O)[C@H](COC1OC(CO)C(O)C(O)C1O)NC(=O)C(c1ccccc1)C1C=CC=CC1] |
| YTC03-22 | α-D-Gal | 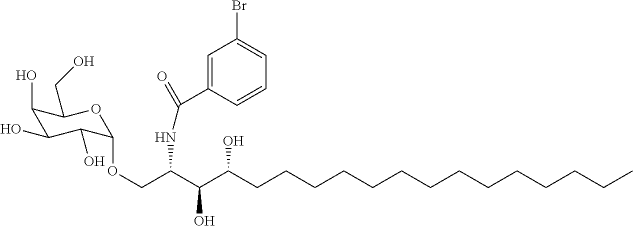 [SMILES: CCCCCCCCCCCCCC[C@@H](O)[C@@H](O)[C@H](COC1OC(CO)C(O)C(O)C1O)NC(=O)c1cccc(Br)c1] |
| YTC03-24 | α-D-Gal | 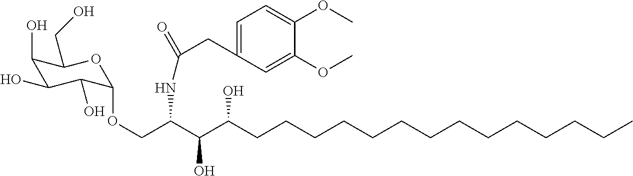 [SMILES: CCCCCCCCCCCCCC[C@@H](O)[C@@H](O)[C@H](COC1OC(CO)C(O)C(O)C1O)NC(=O)Cc1ccc(OC)c(OC)c1] |
| YTC03-25 | α-D-Gal | 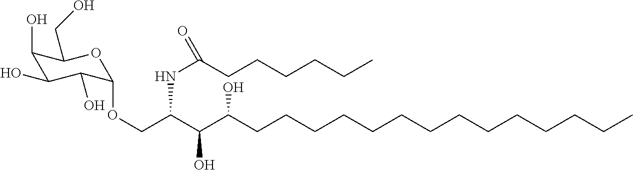 [SMILES: CCCCCCCCCCCCCC[C@@H](O)[C@@H](O)[C@H](COC1OC(CO)C(O)C(O)C1O)NC(=O)CCCCCC] |
| YTC03-30 | α-D-Gal | 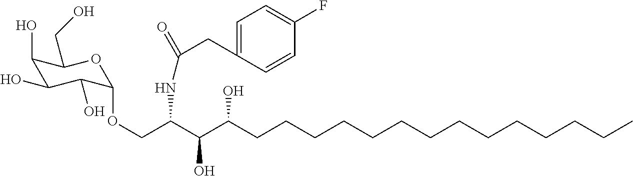 [SMILES: CCCCCCCCCCCCCC[C@@H](O)[C@@H](O)[C@H](COC1OC(CO)C(O)C(O)C1O)NC(=O)Cc1ccc(F)cc1] |
| YTC03-33 | α-D-Gal | 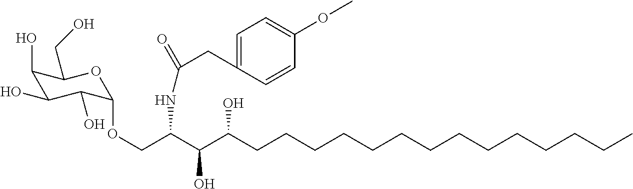 [SMILES: CCCCCCCCCCCCCC[C@@H](O)[C@@H](O)[C@H](COC1OC(CO)C(O)C(O)C1O)NC(=O)Cc1ccc(OC)cc1] |
| YTC03-34 | α-D-Gal | 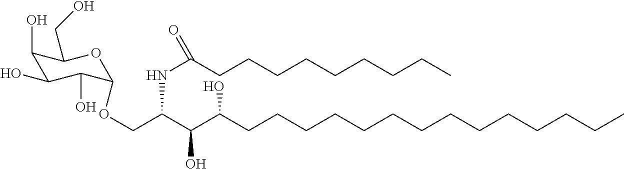 [SMILES: CCCCCCCCCCCCCC[C@@H](O)[C@@H](O)[C@H](COC1OC(CO)C(O)C(O)C1O)NC(=O)CCCCCCCCC] |
| YTC03-35 | α-D-Gal | 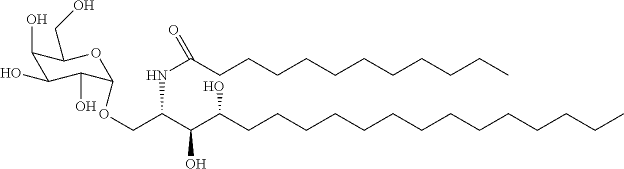 [SMILES: CCCCCCCCCCCCCC[C@@H](O)[C@@H](O)[C@H](COC1OC(CO)C(O)C(O)C1O)NC(=O)CCCCCCCCCCC] |
| YTC03-39 | α-D-Gal | 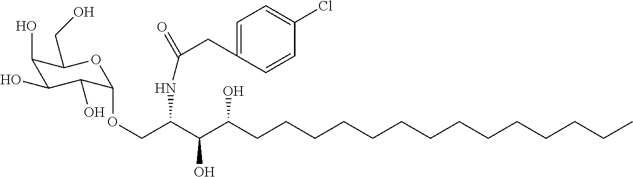 [SMILES: CCCCCCCCCCCCCC[C@@H](O)[C@@H](O)[C@H](COC1OC(CO)C(O)C(O)C1O)NC(=O)Cc1ccc(Cl)cc1] |
| YTC03-41 | α-D-Gal | 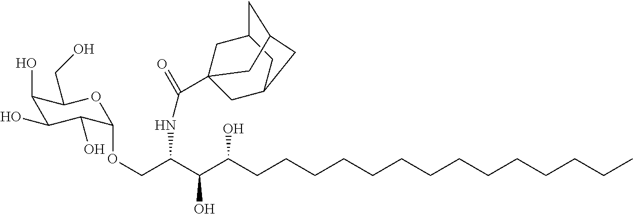 [SMILES: CCCCCCCCCCCCCC[C@@H](O)[C@@H](O)[C@H](COC1OC(CO)C(O)C(O)C1O)NC(=O)C12CC3CC(CC(C3)C1)C2] |
| BF1508-84 | α-D-Gal | 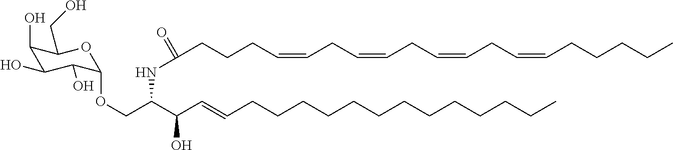 [SMILES: CCCCC/C=C\C/C=C\C/C=C\C/C=C\CCCC(=O)N[C@@H](COC1OC(CO)C(O)C(O)C1O)[C@H](O)/C=C/CCCCCCCCCCCCC] |
| RF03-1 (C- glycoside) | α-D-Gal | 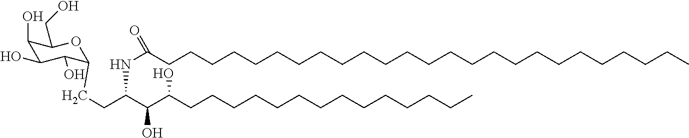 [SMILES: CCCCCCCCCCCCCCCCCCCCCCCCCC(=O)N[C@@H](CCC1OC(CO)C(O)C(O)C1O)[C@H](O)[C@H](O)CCCCCCCCCCCCCC] |

[symbol]

indicates data missing or illegible when filed

In a modified bacterium of the invention, a ceramide-like glycolipid antigen is “physically associated” with a bacterial cell to produce a “modified bacterium.” In certain embodiments, the modified bacterium further comprises a heterologous antigen. By “physically associated” is meant a direct interaction with the bacterial cell, e.g., intercalation of the ceramide-like glycolipid into the plasma membrane or lipid-rich surface of a bacterial cell wall, e.g., a mycobacterial cell wall. In certain embodiments, the ceramide-like glycolipid is physically associated with a bacterial cell wall through non-covalent means. For example, bacterial cells grown in the presence of ceramide-like glycolipid will incorporate the ceramide-like glycolipid into their cell walls. In one aspect of the invention, a ceramide-like glycolipid that is physically associated through non-covalent interactions to a bacterial cell remains extractable from the bacterial cell wall and ceramide-like glycolipid retains its chemical structure and biological activity after extraction. In one embodiment, the method for incorporating a ceramide-like glycolipid into a bacterial cell wall includes culturing the ceramide-like glycolipid with a bacterial cell, e.g., mycobacterial cell, in culture medium that includes a nonionic detergent, e.g., 0.05% TWEEN or Tyloxapol.

In certain embodiments, the modified bacteria of the invention include a bacteria, e.g., a mycobacteria, to which a glycolipid, e.g., a glycolipid that is not a native (or naturally occurring) lipid of the bacterial cell wall, e.g., mycobacterial cell wall, has been stably incorporated. In certain embodiments, the modified bacterium of the invention are prepared by coupling a protection group, e.g., tri-methyl silicate (TMS), to the hydroxyls of a ceramide-like glycolipid; adding a solvent, e.g., petroleum ether (PetEther), to the hydroxyl-protected ceramide-like glycolipid to produce a hydroxyl-protected glycolipid solvent solution, thereafter suspending a bacterium, e.g., a mycobacterium, in the hydroxyl-protected glycolipid solvent solution; and evaporating the solvent, thereby making a ceramide-like glycolipid/mycobacterial complex. This method of the invention may have certain advantages, including, e.g., that the method is highly efficient because the incorporation is performed with a relatively small volume of solvent, and the amount of glycolipid needed is less compared to, e.g., incorporation of glycolipid during mycobacterial growth in culture. Furthermore, the incorporation can be done in a small volume, e.g., a single well, as a controlled step that is separate from bacterial cell growth; thereby allowing for improved standardization and quality control.

As used herein, a “solvent” is a substance used, e.g., a liquid capable of dissolving another substance, to form a solution. In certain aspects of the invention, the solvent of the invention is a nonpolar organic liquid that is capable of dissolving hydrophobic lipids, e.g., synthetic glycolipids; the solvent is volatile so that it can be removed by evaporation; and the solvent is nontoxic to bacteria, e.g., mycobacteria, allowing for the bacteria to be recovered with high viability after glycolipid incorporation. In one embodiment, the solvent is nonpolar. In certain embodiments, the nonpolar solvent is a hexane, a benzene, a toluene, a diethyl ether, a chloroform, an ethyl acetate, a hydrocarbon mixture, or any combination thereof. In certain embodiments, the solvent is petroleum ether, dimethyl sulfoxide (DMSO), benzine, or any combination thereof. In certain embodiments, the solvent of the invention, e.g., petroleum ether, is used to produce a hydroxyl-protected glycolipid solvent solution.

As used herein, a “protection group” or “protecting group” or “protective group” is a group that is introduced into a molecule, e.g., a synthetic glycolipid, by chemical modification of a functional group, e.g., a hydroxyl, to allow for selectivity of subsequent reactions with the original functional group. A protecting group can be removed by deprotection, which removes the protecting group and gives back the original functional group. In certain aspects of the invention, the protection group when coupled to a hydroxyl of a glycolipid, e.g., a synthetic glycolipid, imparts enough hydrophobicity to make the glycolipid soluble in a solvent, e.g., petroleum ether. In another aspect of the invention, the protection group is removable, e.g., subject to deprotection to allow for restoration of the original hydroxyl group, e.g., by spontaneous hydrolysis or an enzyme catalyzed reaction, e.g., within a cell or body fluid.

In one aspect of the invention, the hydroxyl group is deprotected by spontaneous hydrolysis or an enzyme catalyzed reaction within the mycobacterial cell. Studies have show that substitution of the hydroxyl group of a sugar moiety can block activity, and the structure of DC1D/Ligand/TCR complexes derived from X-ray crystallography indicate that modification of hydroxyls will alter or ablate presentation and NKT cell recognition (Borg et al., Nature 448(7149):44-49 (2007). In certain embodiments, adjuvant activity of the ceramide-like glycolipid is restored upon deprotection.

In certain embodiments, the protection group is an alcohol protecting group. In certain embodiments, the protection group is a silyl ether, an acetate, a propionate, a stearate, a acetyl (Ac), a benzoyl (Bz); a benzyl (Bn, Bnl); a beta-methoxyethoxymethyl ether (MEM); a dimethoxytrityl (DMT); a methoxymethyl ether (MOM); a methylthiomethyl either; a pivaloyl (Piv); a tetrahydropyranyl (THP); a trityl (Tr); a methyl rther; a ethoyethyl ether (EE); or any combination thereof. In one embodiment, the protection group is an ester linked aliphatic group, e.g., an acetate, a propionate, or a stearate. In another embodiment, the protection group is a silyl ether, e.g., trimethylsilyl (TMS), tert-butyldimethylsilyl (TBDMS), tert-butyldimethylsilyloxymethyl (TOM), triisopropylsilyl (TIPS) ethers, or any combination thereof. In certain aspects of the invention, the protection group is nontoxic to bacteria, e.g., mycobacteria.

In one aspect of the invention, evaporation is performed by methods know in the art. For example, in certain embodiments, evaporation is performed by directing a stream of nitrogen (or argon) gas onto the surface of the liquid in an open container at room temperature (˜22° C.). In other embodiments, evaporation can be performed by application of mild vacuum and gentle heating. In further embodiments, evaporation can be accelerated by agitation.

Detection of the ceramide-like glycolipid physically associated with the cell wall can be accomplished by methods known to one of skill in the art. By stably binding a ceramide-like glycolipid antigen to a bacterial cell wall, a ceramide-like glycolipid/bacterial complex can be made. In certain embodiments, the compositions of the invention allow for simultaneous administration of a ceramide-like glycolipid antigen and a bacterial cell, e.g., presentation of a glycolipid modified mycobacterial cell to an antigen presenting cell. In certain embodiments, ceramide-like glycolipids are incorporated into a mycobacterial cell wall. The bacterial cell, e.g., mycobacterial cell, can be a killed, live and/or attenuated bacterial cell. In another embodiment, the bacterial cell can be recombinant, e.g., a recombinant bacterial cell expressing a heterologous antigen.

A modified bacterium of the present invention can comprise a single ceramide-like glycolipid antigen, or can comprise heterogeneous mixtures of ceramide-like glycolipid antigens. That is, populations of bacterial cells can be physically associated with a single ceramide-like glycolipid antigen or can be physically associated with to a mixture of ceramide-like glycolipid antigens.

A modified bacterium of the invention, e.g., a ceramide-like glycolipid/bacterial complex of the present invention, or a composition or a vaccine composition comprising the same can be labeled, so as to be directly detectable, or can be used in conjunction with secondary labeled immunoreagents which will specifically bind the compound, e.g., for detection or diagnostic purposes. Labels of interest can include dyes, enzymes, chemiluminescers, particles, radioisotopes, or other directly or indirectly detectable agent. Alternatively, a second stage label can be used, e.g. labeled antibody directed to one of the constituents of the compound of the invention.

Examples of suitable enzyme labels include, but are not limited to malate dehydrogenase, staphylococcal nuclease, delta-5-steroid isomerase, yeast-alcohol dehydrogenase, alpha-glycerol phosphate dehydrogenase, triose phosphate isomerase, peroxidase, alkaline phosphatase, asparaginase, glucose oxidase, beta-gal actosidase, ribonuclease, urease, catalase, glucose-6-phosphate dehydrogenase, glucoamylase, and acetylcholine esterase.

Examples of suitable radioisotopic labels include ³H, ¹¹¹In, ¹²⁵I, ¹³¹I, ³²P, ³⁵S, ¹⁴C, ⁵¹Cr, ⁵⁷To, ⁵⁸Co, ⁵⁹Fe, ⁷⁵Se, ¹⁵²Eu, ⁹⁰Y, ⁶⁷Cu, ²¹⁷Ci, ²¹¹At, ²¹²Pb, ⁴⁷Sc, ¹⁰⁹Pd, etc. Examples of suitable non-radioactive isotopic labels include ¹⁵⁷Gd, ⁵⁵Mn, ¹⁶²Dy, ⁵²Tr, and ⁵⁶Fe.

Examples of suitable fluorescent labels include an ¹⁵²Eu label, a fluorescein label, an isothiocyanate label, a rhodamine label, a phycoerythrin label, a phycocyanin label, an allophycocyanin label, an o-phthaldehyde label, and a fluorescamine label.

Examples of chemiluminescent labels include a luminal label, an isoluminal label, an aromatic acridinium ester label, an imidazole label, an acridinium salt label, an oxalate ester label, a luciferin label, a luciferase label, and an aequorin label.

Examples of nuclear magnetic resonance contrasting agents include heavy metal nuclei such as Gd, Mn, and Fe.

Typical techniques for binding the above-described labels to ceramide-like glycolipids or polypeptides of the invention are provided by Kennedy et al., Clin. Chim. Acta 70:1-31 (1976), and Schurs et al., Clin. Chim. Acta 81:1-40 (1977). Coupling techniques mentioned in the latter are the glutaraldehyde method, the periodate method, the dimaleimide method, the m-maleimidobenzyl-N-hydroxy-succinimide ester method, all of which methods are incorporated by reference herein.

In certain embodiments, a ceramide-like glycolipid comprises a glycosylceramide or analog thereof or an α-galactosylceramide or analog thereof.

In further embodiments, the glycosylceramide or analog thereof comprises Formula I:

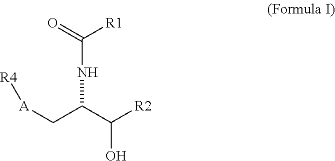

wherein R1 is a linear or branched C₁-C₂₇ alkane or C₂-C₂₇ alkene; or R1 is —C(OH)—R3 wherein R3 is a linear or branched C₁-C₂₆ alkane or C₂-C₂₆ alkene; or R1 is a C₆-C₂₇ alkane or alkene wherein (i) the C₆-C₂₇ alkane or alkene is substituted with a C₅-C₁₅ cycloalkane, C₅-C₁₅ cycloalkene, heterocycle, or aromatic ring or (ii) the C₆-C₂₇ alkane or alkene includes, within the C₆-C₂₇ alkyl or alkenyl chain, a C₅-C₁₅ cycloalkane, C₅-C₁₅ cycloalkene, heterocycle, or aromatic ring;

R2 is one of the following (a)-(e):

—CH₂(CH₂)ₓCH₃, (a)

—CH(OH)(CH₂)ₓCH₃, (b)

—CH(OH)(CH₂)ₓCH(CH₃)₂, (c)

—CH═CH(CH₂)ₓCH₃, (d)

—CH(OH)(CH₂)ₓCH(CH₃)CH₂CH₃, (e)

wherein X is an integer ranging from 4-17;

R4 is an α-linked or a β-linked monosaccharide, or when R1 is a linear or branched C₁-C₂₇ alkane, R4 is:

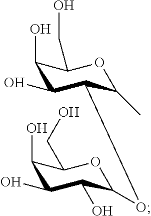

and

A is O or —CH₂.

In another embodiment, the α-galactosylceramide or analog thereof comprises Formula II:

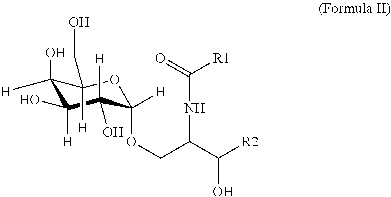

wherein

R1 is a linear or branched C₁-C₂₇ alkane or C₂-C₂₇ alkene; or R1 is —C(OH)—R3 wherein R3 is linear or branched C₁-C₂₆ alkane or C₂-C₂₆ alkene; and

R2 is one of the following (a)-(e):

—CH₂(CH₂)ₓCH₃, (a)

—CH(OH)(CH₂)ₓCH₃, (b)

—CH(OH)(CH₂)ₓCH(CH₃)₂, (c)

—CH═CH(CH₂)ₓCH₃, (d)

—CH(OH)(CH₂)ₓCH(CH₃)CH₂CH₃, (e)

wherein X is an integer ranging from 4-17.

In another embodiment, the α-galactosylceramide or analog thereof comprises Formula III:

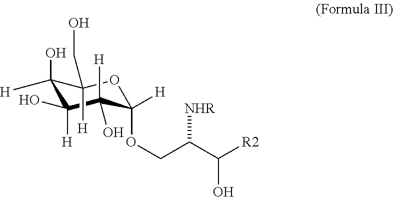

wherein R is H or —C(O)R1, wherein R1 is a linear or branched C₁-C₂₇ alkane or C₂-C₂₇ alkene; or R1 is —C(OH)—R3 wherein R3 is a linear or branched C₁-C₂₆ alkane or C₂-C₂₆ alkene; or R1 is a C₆-C₂₇ alkane or alkene wherein (i) the C₆-C₂₇ alkane or alkene is substituted with a C₅-C₁₅ cycloalkane, C₅-C₁₅ cycloalkene, heterocycle, or aromatic ring or (ii) the C₆-C₂₇ alkane or alkene includes, within the C₆-C₂₇ alkyl or alkenyl chain, a C₅-C₁₅ cycloalkane, C₅-C₁₅ cycloalkene, heterocycle, or aromatic ring; or R1 is a —(CH₂)ₙR5, wherein n is an integer ranging from 0-5, and R5 is —C(O)OC₂H₅, an optionally substituted C₅-C₁₅ cycloalkane an optionally substituted aromatic ring, or an aralkyl, and

R2 is one of the following (a)-(e):

—CH₂(CH₂)ₓCH₃, (a)

—CH(OH)(CH₂)ₓCH₃, (b)

—CH(OH)(CH₂)ₓCH(CH₃)₂, (c)

—CH═CH(CH₂)ₓCH₃, (d)

—CH(OH)(CH₂)ₓCH(CH₃)CH₂CH₃, (e)

wherein X is an integer ranging from 4-17.

In a further embodiment, R1 is selected from the group consisting of

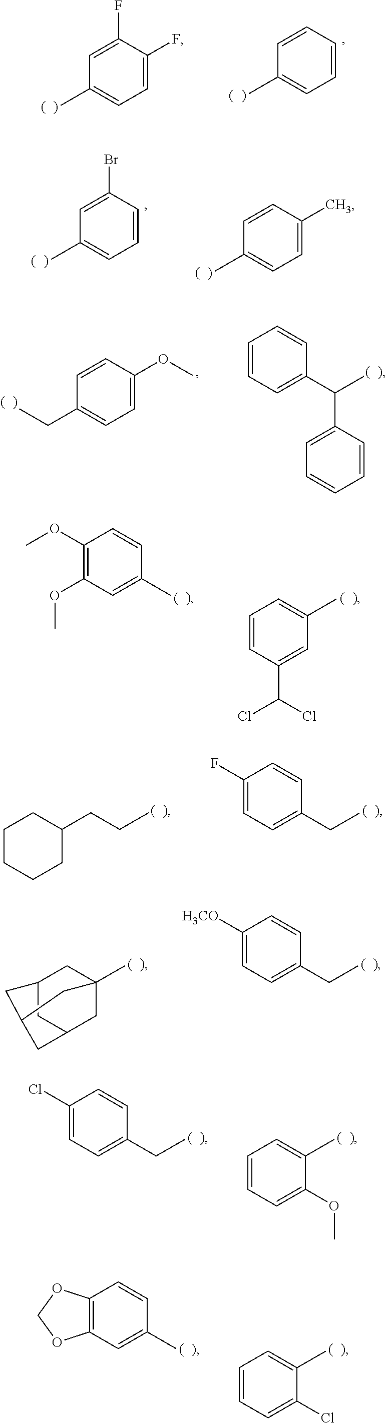

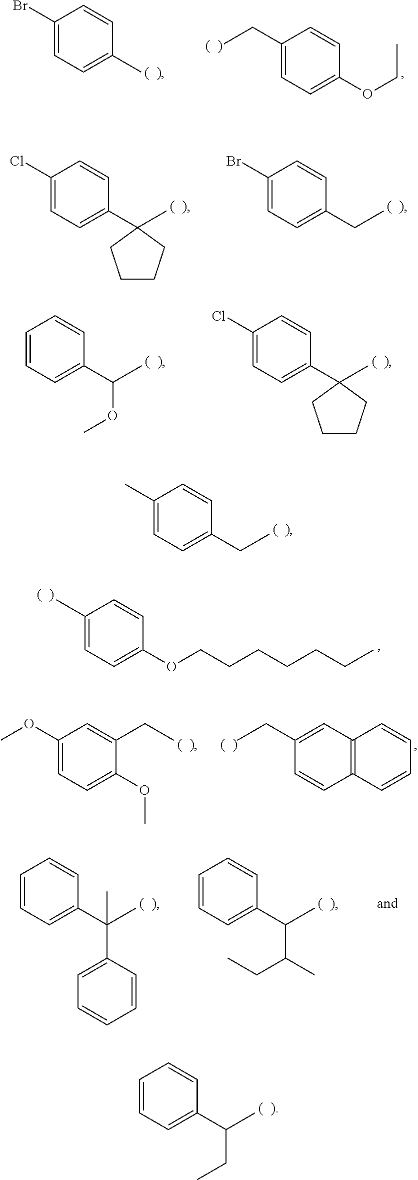

where ( ) represent the point of attachment of R1 to the compound of Formula III.

In another embodiment, the α-galactosylceramide or analog thereof comprises (2S,3S,4R)-1-O-(α-D-galactopyranosyl)-N-hexacosanoyl-2-amino-1,3,4-octadecanetriol (KRN7000) or (2S,3S)-1-O-(α-D-galactopyranosyl)-N-hexacosanoyl-2-amino-1,3-octadecanediol).

In another embodiment, the α-galactosylceramide or analog thereof comprises (2S,3S,4R)-1-CH₂-(α-galactopyranosyl)-N-hexacosanoyl-2-amino-1,3,4-octadecanetriol (α-C-GalCer).

Other non-limiting examples of ceramide-like glycolipids are described in Tsuji et al., U.S. Pat. No. 7,273,852; Taniguchi et al., U.S. Pat. No. 6,531,453; and Higa et al., U.S. Pat. No. 5,936,076, all of which are incorporated herein by reference in their entirety.

### Natural Killer T (NKT) Cells

The natural immune system strikes a complex balance between highly aggressive, protective immune responses to foreign pathogens and the need to maintain tolerance to normal tissues. In recent years there has been increasing recognition that interactions among many different cell types contribute to maintaining this balance. Such interactions can, for example, result in polarized responses with either production of pro-inflammatory cytokines (e.g., interferon-gamma) by TH1 type T cells or production of interleukin-4 (IL-4) by TH2 type T cells that suppress TH1 activity. In a number of different animal models, T cell polarization to TH1 has been shown to favor protective immunity to tumors or infectious pathogens whereas T cell polarization to TH2 can be a critical factor in preventing development of cell-mediated autoimmune disease. The conditions that determine whether immune stimulation will result in aggressive cell-mediated immunity or in down regulation of such responses are highly localized in the sense that each tissue is comprised of a distinctive set of antigen presenting cells (APC) and lymphocyte lineages that interact to favor different immune responses. For example, under optimal conditions, the dendritic cells (DC) localized in a normal tissue can represent predominantly a lineage and stage of maturation that favors tolerogenic interactions and serves as a barrier to cell-mediated autoimmunity whereas a tumor or site of infection will attract mature myeloid dendritic cells that stimulate potent cell-mediated immune responses.

CD1d-restricted NKT cells are a unique class of non-conventional T cells that appear to play an important role in defining the outcome of immune stimulation in the local environment. They share with the larger class of NKT cells the expression of markers of both the T cell and natural killer (NK) cell lineages. As such, NKT cells are considered as part of innate immunity like NK cells and in humans their frequency in normal individuals can be as high as 2.0% of total T lymphocytes (Gumperz et al., J Exp Med 195:625 (2002); Lee et al., J Exp Med 195:637 (2002)).

CD1d-restricted NKT cells are distinguished from other NKT cells by their specificity for lipid and glycolipid antigens presented by the monomorphic MHC class Ib molecule, CD1d (Kawano et al., Science 278:1626-1629 (1997)). CD1d is a non-MHC encoded molecule that associates with β2-microglobulin and is structurally related to classical MHC class I molecules. CD has a hydrophobic antigen-binding pocket that is specialized for binding the hydrocarbon chains of lipid tails or hydrophobic peptides. Zeng et al., Science 277: 339-345, (1997). CD1d is known to bind a marine sponge derived α-glycosylated sphingolipid, α-galactosylceramide (α-GalCer), and related molecules such as ceramide-like glycolipid antigens with α-linked galactose or glucose but not mannose. Kawano et al., Science 278:1626-1629 (1997); and Zeng et al., Science 277: 339-345 (1997). As discussed herein, the ability to activate CD1d-restricted NKT cells by stimulation with α-GalCer or related molecules bound to CD1d of antigen presenting cells has greatly facilitated functional analysis of this non-conventional T cell subset. In the absence of inflammation, CD1d-restricted NKT cells have been shown to localize preferentially in certain tissues like thymus, liver and bone marrow (Wilson et al., Trends Mol Med 8:225 (2002)) and antitumor activity of NKT cells has been mainly investigated in mouse liver metastasis.

NKT cells have an unusual ability of secreting both TH1 and TH2 cytokines and potent cytotoxic as well as regulatory functions have been documented in inflammation, autoimmunity and tumor immunity (Bendelac et al., Science 268:863 (1995); Chen and Paul, J Immunol 159:2240 (1997); and Exley et al., J Exp Med 186:109 (1997)).

Among the CD1d-restricted NKT cells is a subset, referred to herein as “iNKT cells,” that express a highly conserved αβT cell receptor (TCR). In humans this invariant TCR is comprised of Vα24Jα15 in association with Vβ11 whereas in mice the receptor comprises the highly homologous Vα14Jα18 and Vβ8.2. Other CD1d-restricted NKT cells express more variable TCR. Both TCR invariant and TCR variant classes of CD1d-restricted T cells can be detected by binding of CD1d-tetramers loaded with α-GalCer (Benlagha et al., J Exp Med 191:1895-1903 (2000); Matsuda et al., J Exp Med 192:741-754 (2000); and Karadimitris et al., Proc Natl Acad Sci USA 98:3294-3298 (2001)). CD1d-restricted NKT cells, as defined in this application (CD1d-restricted NKT), include cells that express either invariant or variant TCR and that bind or are activated by CD1d loaded with either α-GalCer or with related ceramide-like glycolipid antigens. CD1d-restricted NKT cells, as defined in this application (CD1d-NKT), include cells that express either invariant or variant TCR and that bind or are activated by CD1d loaded with either α-GalCer or with related sphingolipids that have α-linked galactose or glucose including molecules such as OCH, which differs from α-GalCer by having a shortened long-chain sphingosine base (C5 vs. C14) and acyl chain (C24 vs. C26) (Miyamoto et al., Nature 413:531-4 (2001)).

CD1d-restricted NKT have been shown to have direct cytotoxic activity against targets that express CD1d. It is likely, however, that the effect of CD1d-restricted NKT on immune responses is amplified through recruitment of other lymphocytes either by direct interaction or, perhaps even more importantly, by indirect recruitment through interaction with DC. CD1d-restricted NKT have the unique ability to secrete large quantities of IL-4 and IFN-γ early in an immune response. Secretion of IFN-γ induces activation of DC which produce interleukin-12 (IL-12). IL-12 stimulates further IFN-γ secretion by NKT cells and also leads to activation of NK cells which secrete more IFN-γ.

Since CD1d-restricted NKT are able to rapidly secrete large amounts of both IL-4 and IFN-γ, the polarization of immune responses will depend on whether the effect of pro-inflammatory IFN-γ or anti-inflammatory IL-4 cytokines predominate. This has been reported to be, in part, a function of the relative frequency of different subsets of CD1d-restricted NKT. These subsets include (i) an invariant CD1d-restricted NKT population that is negative for both CD4 and CD8 and that gives rise to predominantly a TH1 type response including secretion of pro-inflammatory IFN-γ and INF-α and (ii) a separate population of CD1d-restricted NKT that is CD4+ and that gives rise to both a TH1 type and TH2 type response including secretion c f the anti-inflammatory Th2-type cytokines IL-4, IL-5, IL-10 and IL-13 (Lee et al., J Exp Med 195:637-41 (2002); and Gumperz et al., J Exp Med 195:625-36 (2002)). In addition, NKT cell activity is differentially modulated by depending on the particular ceramide-like glycolipid bound to CD1d (see, e.g., US Patent Application Publication No. 2006/0052316). Local factors that influence activation of CD1d-restricted NKT subsets include the cytokine environment and, importantly, the DC that are recruited to that environment.

A family of ceramide-like glycolipids (i.e., α-galactosylceramide (α-GalCer) and related α-glycosyl ceramides), have been shown stimulate strong CD1d-restricted responses by murine NKT cells (Kawano et al., 1997). These compounds contain an α-anomeric hexose sugar (galactose or glucose being active for NKT cell recognition), distinguishing them from the ceramides that commonly occur in mammalian tissues which contain only β-anomeric sugars. These compounds are known to occur naturally in marine sponges, the source from which they were originally isolated, and became of interest to immunologists when it was demonstrated that α-GalCer induced dramatic tumor rejection as a result of immune activation when injected into tumor bearing mice (Kobayashi et al., Oncol. Res 7:529-534 (1995)). Subsequently, this activity was linked to the ability of α-GalCer to rapidly activate NKT cells through a CD1d dependent mechanism. It has now been shown that α-GalCer binds to CD1d, thus creating a molecular complex that has a measurable affinity for the TCRs of NKT cells (Naidenko et al., J Exp. Med. 190:1069-1080 (1999); Matsuda et al., J Exp. Med. 192:741 (2000); Benlagha et al., J Exp. Med. 191:1895-1903 (2000)). Thus, α-GalCer provides a potent agent that can enable activation of the majority of NKT cells both in vitro and in vivo.

The most extensively studied NKT activating α-GalCer, called KRN7000 in the literature, is a synthetic molecule that has a structure similar to natural forms of α-GalCer that were originally isolated from a marine sponge on the basis of their anti-cancer activity in rodents (Kawano et al., Science 278:1626-1629 (1997); Kobayashi et al., 1995; Iijima et al., Bioorg. Med. Chem. 6:1905-1910 (1998); Inoue et al., Exp. Hematol. 25:935-944 (1997); Kobayashi et al., Bioorg. Med. Chem. 4:615-619 (1996a) and Biol. Pharm. Bull. 19:350-353 (1996b); Uchimura et al., Bioorg. Med. Chem. 5:2245-2249 (1997a); Uchimura et al., Bioorg. Med. Chem. 5:1447-1452 (1997b); Motoki et al., Biol. Pharm. Bull. 19:952-955 (1996a); Nakagawa et al., Oncol. Res. 10:561-568 (1998); Yamaguchi et al., Oncol. Res. 8:399-407 (1996)). One synthetic analogue of KRN7000 with a truncated sphingosine base showed an enhanced ability to suppress autoimmunity in a mouse model of experimental allergic encephalomyelitis (EAE) (Miyamoyo et al., Nature 413:531-524 (2001)). Other variants altered in the α-GalCer sphingosine base are identified in U.S. Pat. No. 5,936,076.

A large body of literature dating from November 1997 to the present time has studied the mechanism by which KRN7000 activates the immune system of mammals (Kawano et al., Science 278:1626-1629 (1997); Benlagha et al., J Exp. Med. 191:1895-1903 (2000); Burdin et al., Eur. J Immunol. 29:2014-2025 (1999); Crowe et al., J. Immunol. 171:4020-4027 (2003); Naidenko et al., J Exp. Med. 190:1069-1080 (1999); Sidobre et al., J. Immunol. 169:1340-1348 (2002); Godfrey et al., Immunol. Today 21:573-583 (2000); Smyth and Godfrey, Nat. Immunol. 1:459-460 (2000)). These studies uniformly show that the proximal mechanism for the effect of KRN7000 is the binding of this compound to a CD1d protein, which is expressed on most hematopoietic cells, as well as some epithelial and other cell lineages. The binding of KRN7000 to CD1d creates a molecular complex that is recognized with high affinity by the T cell antigen receptors (TCRs) of a subset of T lymphocytes called natural killer T cells (NKT cells). Recognition of the KRN7000/CD1d complex leads to rapid activation of the NKT cells, which reside in the liver, spleen and other lymphoid organs and have the potential to traffic to potentially any tissue. Activated NKT cells rapidly secrete a wide range of chemokines and other cytokines, and also have the capability of activating other cell types such as dendritic cells and natural killer (NK) cells. The chain of events that follows the activation of NKT cells by KRN7000/CD1d complexes has been shown to have many potential downstream effects on the immune system. For example, in the setting of certain types of infections this can lead to an adjuvant effect that boosts the adaptive immunity to the infection and promotes healing. Or, in the setting of certain types of autoimmune diseases, the activation of NKT cells by KRN7000 can alter the course of the autoimmune response in a way that suppresses tissue destruction and ameliorates the disease.

The functions of NKT lymphocytes remain incompletely resolved, but a variety of studies point to an important role for these T cells in the regulation of immune responses. A hallmark of NKT cells is their rapid production of large quantities of both IL-4 and IFN-γ upon stimulation through their α-PTCRs (Exley et al., J. Exp. Med. 186:109 (1997). In fact, their identification as perhaps the major cell responsible for the early production of IL-4 during immune activation suggested that they may play a critical role in polarizing type 2 (Th2) T cell responses. In this regard, it is not surprising that NKT cells have been identified to play a significant role in determining the outcome of infections with a variety of different pathogens in mice.

A number of indirect mechanisms contribute to the protective effect of CD1d-restricted NKT cells. Activation of NKT cells by administration of α-GalCer in vivo results in concomitant activation of NK cells (Eberl and MacDonald, Eur. J. Immunol. 30:985-992 (2000); and Carnaud et al., J. Immunol. 163:4647-4650 (1999)). In mice deficient in NKT cells, α-GalCer is unable to induce cytotoxic activity by NK cells. NKT cells also enhance the induction of classical MHC class I restricted cytotoxic T cells (Nishimura et al., Int Immunol 12:987-94 (2000); and Stober et al., J Immunol 170:2540-8 (2003)).

The availability of a defined antigen, e.g., α-GalCer and related antigens, that can be employed to specifically activate CD1d-restricted NKT cells has made it possible to examine the role of these non-conventional T cells in a variety of immune responses.

Alpha-GalCer administration has an effect on a number of different microbial infections, including protective effects in murine malaria, fungal and hepatitis B virus infections. Kakimi et al., J Exp Med 192:921-930 (2000); Gonzalez-Aseguinolaza et al., Proc Natl Acad Sci USA 97:8461-8466 (2000); and Kawakami et al., Infect Immun 69:213-220 (2001). Dramatic effects of administration of α-GalCer have also been observed in animal models of tumor immunity. For example, stimulation with α-GalCer suppresses lung and liver metastases in an NKT dependent manner (Smyth et al., Blood 99:1259 (2002)). In addition, α-GalCer has been shown to have a protective effect against certain autoimmune diseases, including type 1 diabetes and experimental autoimmune encephalomyelitis (EAE, a well-known murine model system for multiple sclerosis). Hong S et al. Nat. Med. 7:1052-1056 (2001) and Miyamoto K. et al., Nature 413:531-534 (2001).

### NKT Activity Assays

The ability of a composition of the present invention to modulate an immune response can be readily determined by an in vitro assay. NKT cells for use in the assays include transformed NKT cell lines, or NKT cells which are isolated from a mammal, e.g., from a human or from a rodent such as a mouse. NKT cells can be isolated from a mammal by sorting cells that bind CD1d:α-GalCer tetramers. See, for example, Benlagha et al., J Exp Med 191:1895-1903 (2000); Matsuda et al., J Exp Med 192:741-754 (2000); and Kaiadimitris et al., Proc Natl Acad Sci USA 98:3294-3298 (2001). A suitable assay to determine if a compound or composition of the present invention is capable of modulating the activity of NKT cells is conducted by co-culturing NKT cells and antigen presenting cells, adding the particular compound or composition of interest to the culture medium that targets either the antigen presenting cells or the NKT cells directly, and measuring IL-4 or IFN-γ production. A significant increase or decrease in IL-4 or IFN-γ production over the same co-cult ure of cells in the absence of the compound or composition of the invention or in the presence of a compound or composition of the invention with a non-targeting antibody indicates stimulation or inhibition of NKT cells.

The NKT cells employed in the assays are incubated under conditions suitable for proliferation. For example, an NKT cell hybridoma is suitably incubated at about 37° C. and 5% CO2 in complete culture medium (RPMI 1640 supplemented with 10% FBS, penicillin/streptomycin, L-glutamine and 5×10⁻⁵ M 2-mercaptoethanol). Serial dilutions of the compound can be added to the NKT cell culture medium. Suitable concentrations of the compound added to the NKT cells typically will be in the range of from 10⁻¹² to 10⁻⁶ M. Use of antigen dose and APC numbers giving slightly submaximal NKT cell activation can be used to detect stimulation or inhibition of NKT cell responses by the compounds of the invention.

Alternatively, rather than measurement of an expressed protein such as IL-4 or IFN-γ, modulation of NKT cell activation can be determined by changes in antigen-dependent T cell proliferation as measured by radiolabelling techniques as are recognized in the art. For example, a labeled (e.g., tritiated) nucleotide can be introduced to an assay culture medium.

Incorporation of such a tagged nucleotide into DNA serves as a measure of T cell proliferation. This assay is not suitable for NKT cells that do not require antigen presentation for growth, e.g., NKT cell hybridomas. A difference in the level of T cell proliferation following contact with the compound or composition of the invention indicates the complex modulates activity of the T cells. For example, a decrease in NKT cell proliferation indicates the compound or composition can suppress an immune response. An increase in NKT cell proliferation indicates the compound or composition can stimulate an immune response.

Additionally, the ⁵¹Cr release assay can be used to determine cytotoxic activity.

These in vitro assays can be employed to select and identify ceramide-like glycolipid/bacterial cell complexes and compositions comprising same that are capable of appropriately modulating an immune response. Assays described above, e.g., measurement of IL-4 or IFN-γ production or NKT cell proliferation, are employed to determine if contact with the compound modulates T cell activation.

In addition or alternatively, immunization challenge experiments in animals, e.g., mice, rabbits, non-human primates, can be used to identify ceramide-like glycolipid/bacterial cell complexes and compositions comprising same that are capable of appropriately modulating an immune response and that may be efficacious for treatment and/or prevention of bacterial diseases, e.g., tuberculosis, in humans. For example, mice can be vaccinated with ceramide-like glycolipid/bacterial cell complex, e.g., BCG/αGalCer or BCG/α-C-GalCer (e.g., 5×10⁶ CFU/mouse) and challenged with an infectious bacteria, e.g., virulent strain M. tuberculosis H37Rv.

### Methods of Treatment

A modified bacterium, composition, or vaccine composition of the present invention can be used both to prevent a disease, and also to therapeutically treat a disease, e.g., a viral disease, a bacterial disease, a fungal disease, a parasitic disease, an allergic disease, or a proliferative diseases, e.g., cancer. In individuals already suffering from a disease, the present invention is used to further stimulate or modulate the immune system of the animal, thus reducing or eliminating the symptoms associated with that disease or disorder. As defined herein, “treatment” refers to the use of one or more modified bacteria, compositions, or vaccine compositions of the present invention to prevent, cure, retard, or reduce the severity of given disease symptoms in an animal, and/or result in no worsening of the disease over a specified period of time in an animal which has already contracted the disease and is thus in need of therapy.

The term “prevention” or “prevent” refers to the use of one or more modified bacteria, compositions, or vaccine compositions of the present invention to generate immunity in an animal which has not yet contracted a disease, thereby preventing or reducing disease symptoms if the animal is later disposed to develop that disease. The methods of the present invention therefore can be referred to as therapeutic methods or preventative or prophylactic methods. It is not required that any modified bacterium, composition, or vaccine composition of the present invention provide total immunity to a disease agent or totally cure or eliminate all disease symptoms.

As used herein, an “animal in need of therapeutic and/or preventative immunity” refers to an individual for whom it is desirable to treat, i.e., to prevent, cure, retard, or reduce the severity of certain disease symptoms, and/or result in no worsening of disease over a specified period of time.

An “effective amount” is that amount the administration of which to an individual, either in a single dose or as part of a series, is effective for treatment and/or prevention. An amount is effective, for example, when its administration results in a reduced incidence or severity of disease symptoms associated with M. tuberculosis relative to an untreated individual, as determined about two weeks after challenge with infectious M. tuberculosis. This amount varies depending upon the health and physical condition of the individual to be treated, the taxonomic group of individual to be treated (e.g. human, nonhuman primate, primate, etc.), the responsive capacity of the individual's immune system, the degree of protection desired, the formulation of the vaccine, a professional assessment of the medical situation, and other relevant factors. It is expected that the effective amount will fall in a relatively broad range that can be determined through routine trials.

The term “vertebrate” is intended to encompass a singular “vertebrate” as well as plural “vertebrates” and comprises mammals and birds, as well as fish, reptiles, and amphibians.

The teen “mammal” is intended to encompass a singular “mammal” and plural “mammals,” and includes, but is not limited to humans; primates such as apes, monkeys (e.g., owl, squirrel, cebus, rhesus, African green, patas, cynomolgus, and cercopithecus), orangutans, baboons, gibbons, and chimpanzees; canids such as dogs and wolves; felids such as cats. lions, and tigers; equines such as horses, donkeys, and zebras, food animals such as cows, pigs, and sheep; ungulates such as deer and giraffes; ursids such as bears; and others such as rabbits, mice, ferrets, seals, whales. In particular, the mammal can be a human subject, a food animal or a companion animal.

The term “bird” is intended to encompass a singular “bird” and plural “birds,” and includes, but is not limited to feral water birds such as ducks, geese, terns, shearwaters, and gulls; as well as domestic avian species such as turkeys, chickens, quail, pheasants, geese, and ducks. The term “bird” also encompasses passerine birds such as starlings and budgerigars.

The invention provides methods of preventing or treating a disease in an animal in need of such treatment or prevention, comprising administering to an animal with that disease, or prone to contract that disease, a composition comprising a bacterial cell, e.g., a mycobacterial cell, and a ceramide-like glycolipid antigen wherein said ceramide-like glycolipid is incorporated into the cell wall of the bacterial cell as described herein. In farther embodiments, the bacterial cell can be used as as an antigen carrier for delivery of heterologous antigens from another pathogen or a tumor specific antigen.

The present invention also includes a method of modulating, i.e., either stimulating or inhibiting an immune response, comprising administering to an animal an effective amount of a composition comprising a bacterial cell, e.g., a mycobacterial cell, and a ceramide-like glycolipid, wherein said ceramide-like glycolipid is incorporated into the cell wall of the bacterial cell as described herein. In further embodiments, the composition further comprises a heterologous antigen from another pathogen or a tumor specific antigen, and the immune response is a priming immune response against the heterologous antigen.

In certain embodiments, the methods of the invention include treating a disease, e.g., an infectious or proliferative disease, in an animal with the disease by administering to the animal with the disease a composition of the invention, e.g., a modified mycobacterium of the invention, in an amount sufficient to alter the progression of said disease.

In other embodiments, the methods of the invention include preventing a disease, e.g., an infectious or proliferative disease, in an animal in need of prevention of the disease by administering to the animal in need thereof a composition of the invention, e.g., a modified mycobacterium of the invention, in an amount sufficient to enhance an immune response against the bacterium or antigen encoded by the bacterium relative to administration of an unmodified bacterial cell lacking the ceramide-like glycolipid.

In further embodiments, the disease being treated or prevented can be, without limitation a viral, bacterial, fungal, or parasitic infectious disease, an allergy or a proliferative disease such as cancer. More specifically, the disease can be, e.g., tuberculosis, Hansen's disease, pulmonary disease resembling tuberculosis, lymphadenitis, skin disease, disseminated disease, bubonic plague, pneumonic plague, tularemia, Legionnaire's disease, anthrax, typhoid fever, paratyphoid fever, foodborne illness, listeriosis, malaria, HIV, SIV, HPV, RSV, influenza, hepatitis (HAV, HBV, and HCV).

In another embodiment the methods of the invention include enhancing an immune response to a bacterial cell, e.g., a mycobacterial cell, in an animal, comprising administering to the animal a modified bacterium of the invention, e.g., ceramide-like glycolipid incorporated into the cell wall of a bacterial cell; and wherein the modified bacterium is administered in an amount sufficient to enhance antigen specific CD8 T-cell responses against an antigen and enhance the activity of Natural Killer T (NKT) cells in said animal.

In another embodiment the methods of the invention include simultaneous administration of a ceramide-like glycolipid adjuvant and a bacterial cell, e.g., mycobacterial cell, to an antigen presenting cell by stably binding a ceramide-like glycolipid adjuvant to the cell wall of the bacterial cell to make a ceramide-like glycolipid/bacterial complex; and then administering the ceramide-like glycolipid/bacterial complex to the antigen presenting cell. In certain embodiments, the ceramide-like glycolipid/bacterial complex further comprises a heterologous antigen.

As used herein, an “subject in need thereof” refers to an individual for whom it is desirable to treat, i.e., to prevent, cure, retard, or reduce the severity of the symptoms of a disease, e.g., a bacterial infection, and/or result in no worsening of a disease over a specified period of time.

According to these methods, a modified bacterium, composition, or vaccine composition if the present invention can be administered in an amount sufficient to alter the progression of a disease.

“Immunization” (administration of a vaccine) is a common and widespread procedure and the vaccines of the invention used can be essentially any preparation intended for active immunological prophylaxis, including without limitation preparations of killed microbes of virulent strains and living microbes of attenuated strains. Stedman's Illustrated Medical Dictionary (24th edition), Williams & Wilkins, Baltimore, p. 1526 (1982). In some cases, vaccines must be administered more than once in order to induce effective protection; for example, known anti-toxin vaccines must be given in multiple doses.

The terms “priming” or “primary” and “boost” or “boosting” as used herein to refer to the initial and subsequent immunizations, respectively, i.e., in accordance with the definitions these terms normally have in immunology. However, in certain embodiments, e.g., where the priming component and boosting component are in a single formulation, initial and subsequent immunizations may not be necessary as both the “prime” and the “boost” compositions are administered simultaneously. See also, McShane H, Curr Opin Mol Ther 4(1):13-4 (2002) and Xing Z and Charters T J, Expert Rev Vaccines 6(4):539-46 (2007), both incorporated herein by reference.

In certain embodiments, one or more compositions of the present invention are utilized in a “prime boost” regimen. In certain embodiments, one or more vaccine compositions of the present invention are delivered to a vertebrate, thereby priming the immune response of the vertebrate to a bacterial antigen, e.g., a mycobacterial antigen, and then a second immunogenic composition is utilized as a boost vaccination. In certain embodiments, one or more vaccine compositions of the present invention are delivered to a vertebrate, thereby priming the immune response of the vertebrate to a heterologous antigen, e.g., a heterologous antigen carried by a glycolipid modified bacteria, and then a second immunogenic composition is utilized as a boost vaccination. In another embodiment, one or more vaccine compositions of the present invention are used to prime immunity, and then a second immunogenic composition, e.g., a recombinant bacterial vaccine, is used to boost the anti-bacterial immune response. The vaccine compositions can comprise one or more vectors for expression of one or more genes that encode immunogenic polypeptides as described herein.

The present invention further provides a method for generating, enhancing, or modulating a protective and/or therapeutic immune response to a pathogen, e.g., a bacterial, fungal, viral, or parasitic pathogen, or a tumor antigen, in a vertebrate, comprising administering to a vertebrate in need of therapeutic and/or preventative immunity one or more of the modified bacterium, compositions, or vaccine compositions described herein. In this method, the composition includes a modified bacterium, e.g., a mycobacterium comprising a ceramide-like glycolipid incorporated into its cell wall. In certain embodiments, the modified bacterium further comprises a heterologous antigen.

In certain embodiments, the modified bacterium, composition, or vaccine composition of the invention, e.g., BCG/αGalCer or BCG/α-C-GalCer can be used to reduce the dose required to obtain a favorable response to the vaccine. This would have the potential benefits of reducing local and systemic toxicity, thus increasing the safety profile of the vaccine. In addition, this could have the benefit of allowing for reduced cost of production.

Certain embodiments of the present invention include a method of reducing or eliminating the anergic response of NKT cells to multiple administrations of ceramide-like glycolipid antigens administered by themselves, which are therefore presented to NKT cells in the context of a bacterial cell wall. It has been shown that multiple administrations of α-GalCer, administered by itself, causes NKT cells to become non-responsive for an extended period of time. The present invention, in which glycolipids such as α-GalCer are administered as part of a ceramide-like glycolipid/bacterial cell complex, may protect NKT cells from anergy in response to antigen, and allow for a prolonged response upon multiple administrations. Accordingly, NKT cells are activated in response to stimulation with ceramide-like glycolipid/bacterial cell complexes loaded with a ceramide-like glycolipid antigen of the present invention and furthermore, NKT cells can be reactivated in response to restimulation by ceramide-like glycolipid/bacterial cell complexes loaded with a ceramide-like glycolipid antigen of the present invention.

According to the methods of the present invention, a composition comprising a bacterial cell and a ceramide-like glycolipid antigen as described herein is administered to modulate an immune response in an animal, e.g., a vertebrate, e.g., a mammal, e.g., a human. In certain embodiments, the methods of the present invention result in the enhancement of an immune response, e.g., to an immunogen delivered before, after, or concurrently with a ceramide-like glycolipid/bacterial cell complex. Administration of ceramide-like glycolipid/bacterial cell complexes of the invention, e.g., with an immunogen, may typically result in the release of a cytokines from immune cells, e.g., NKT cells or NK cells. Cytokines released in response to administration of a modified bacterium, composition, or vaccine composition of the invention may be those associated with a TH1-type immune response, e.g., interferon gamma and TNF-alpha. Alternatively, or in addition, administration of a modified bacterium, composition, or vaccine composition of the present invention may result in the release of cytokines associated with a TH2-type immune response, e.g., IL-4, IL-5, IL-10, or IL-13. Alternatively, or in addition, administration of a modified bacterium, composition, or vaccine composition of the present invention may result in the release of other cytokines, e.g., IL-2, IL-1β, IL-12, IL-17, IL-23, TNF-β/LT, MCP-2, oncostatin-M, and RANTES. Methods to modulate the type of cytokines released include varying the ceramide-like glycolipid antigen of the ceramide-like glycolipid/bacterial cell complex. Choosing and testing various ceramide-like glycolipid antigens for their effect on cytokine release from NKT or other immune cells can be performed using in vitro assays described elsewhere herein and in Porcelli, U.S. Patent Appl. Publ. No. 2006/0052316, as well as by additional methods well-known by those of ordinary skill in the art. Administration of ceramide-like glycolipid/bacterial cell complexes of the present invention and vaccine compositions comprising same may further modulate an immune response by inducing proliferation of NKT cells, and also by inducing recruitment and or activation of other immune cells including, but not limited to NK cells, CTLs, other T lymphocytes, e.g., CD8+ or CD4+ T lymphocytes, dendritic cells, B lymphocytes, and others.

In certain embodiments, administration of ceramide-like glycolipid/bacterial cell complexes of the present invention and compositions comprising same affects one or more NKT cell activities such as, but not limited to cell proliferation, the production of one or more cytokines, or recruitment and/or activation of non-NKT immune system cells including, but not limited to NK cells, CTLs, other T lymphocytes, e.g., CD8+ or CD4+ T lymphocytes, dendritic cells, B lymphocytes, and others.

Certain embodiments of the present invention involve the use of ceramide-like glycolipid/bacterial cell complexes of the invention as recombinant vaccines used to modulate an immune response to an immunogen, e.g., a pathogen antigen or tumor antigen, that is expressed by the bacterial cell/ceramide-like glycolipid complex. Accordingly, the present invention provides a method of inducing an immune response to an immunogen in an animal, where the method comprises administering to an animal in need thereof a composition comprising an immunogen, which is present in a ceramide-like glycolipid/bacterial cell complex. According to this embodiment, the ceramide-like glycolipid/bacterial cell complex is administered in an amount sufficient to induce the immune response against the immunogen, e.g., bacterial pathogen or immunogen expressed by the recombinant bacteria, relative to administration of the immunogen without the ceramide-like glycolipid/bacterial cell complex. A ceramide-like glycolipid/bacterial cell complex for use as an vaccine can in certain embodiments be a recombinant bacterial cell that presents a recombinant antigen. In other embodiments, the immune response is to the bacterial cell of the ceramide-like glycolipid/bacterial cell complex. In other embodiments, a ceramide-like glycolipid/bacterial cell complex for use as an vaccine can be targeted to a particular organ, tissue, cell or cell surface marker as described, e.g., in Bruno et al. U.S. Patent Appl. Publ. No. 2006/0269540.

In certain embodiments, ceramide-like glycolipid/bacterial cell complexes of the present invention and compositions comprising same are administered as a therapeutic vaccine, e.g., to an animal already suffering from a disease. According to these methods, the immune response elicited by a modified bacterium of the invention is effective in treating, e.g., affecting the outcome of the disease by reducing symptoms or lessening the severity of the disease, and the ceramide-like glycolipid/bacterial cell complex is administered in an amount sufficient to modulate the immune response against the immunogen relative to administration of the immunogen in the absence of the ceramide-like glycolipid/bacterial cell complex. Alternatively, ceramide-like glycolipid/bacterial cell complexes of the present invention and compositions comprising same are administered as a prophylactic vaccine, i.e., to prevent, or reduce symptoms to a disease, such as an infectious disease that might be contracted by that animal in the future. According to these methods, the immune response elicited by the ceramide-like glycolipid/bacterial cell complexes is effective in preventing, e.g., affecting the outcome of the disease by reducing symptoms or lessening the severity of the disease, and the ceramide-like glycolipid/bacterial cell complex is administered in an amount sufficient to modulate the immune response against the immunogen relative to administration of the immunogen in the absence of the ceramide-life glycolipid/bacterial cell complex.

The present invention also provides ceramide-like glycolipid/bacterial cell complex compositions for use in the methods described herein. Such compositions comprise a bacterial cell and a ceramide-like glycolipid as described elsewhere herein. For example, ceramide-like glycolipid/bacterial cell complex compositions of the present invention can include ceramide-like glycolipid/mycobacterial cell complex, e.g, αGalCer/BCG and α-C-GalCer/BCG. In certain embodiments, the ceramide-like glycolipid/mycobacterial cell complex of the invention is produced by the methods described elsewhere herein.

The methods, modified bacteria, compositions, or vaccine compositions as described herein are also useful for raising an immune response against infectious agents, e.g., a ceramide-like glycolipid/bacterial cell complex wherein the bacterial cell of the complex expresses a heterologous antigen, e.g., a viral antigen, a bacterial antigen, a fungal antigen, or a parasitic antigen. Infectious agents that can cause disease or symptoms that can be treated by the methods, modified bacteria, compositions, or vaccine compositions of the invention include, but are not limited to viral, bacterial, fungal, and parasitic agents. Examples of viruses, include, but are not limited to the following DNA and RNA viral families: Arbovirus, Adenoviridae, Arenaviridae, Arterivirus, Birnaviridae, Bunyaviridae, Caliciviridae, Circoviridae, Coronaviridae, Flaviviridae, Hepadnaviridae (hepatitis), Herpesviridae (such as, Cytomegalovirus, Herpes Simplex, Herpes Zoster), Mononegavirus (e.g., Paramyxoviridae, Morbillivirus, Rhabdoviridae), Orthomyxoviridae (e.g., Influenza), Papovaviridae, Parvoviridae, Picornaviridae, Poxyiridae (such as Smallpox or Vaccinia), Reoviridae (e.g., Rotavirus), Retroviridae (HTLV-I, HTLV-II, Lentivirus), and Togaviridae (e.g., Rubivirus). Viruses falling within these families can cause a variety of diseases or symptoms, including, but not limited to: arthritis, bronchiollitis, encephalitis, eye infections (e.g., conjunctivitis, keratitis), chronic fatigue syndrome, hepatitis (A, B, C, E, Chronic Active, Delta), meningitis, opportunistic infections (e.g., AIDS), pneumonia, Burkitt's Lymphoma, chickenpox, hemorrhagic fever, measles, mumps, parainfluenza, rabies, the common cold, Polio, leukemia, Rubella, sexually transmitted diseases, skin diseases (e.g., Kaposi's, warts), and viremia.

Similarly, bacterial or fungal agents that can cause disease or symptoms can be treated or prevented by the methods, modified bacterium, compositions, or vaccine compositions of the invention. These include, but are not limited to, the following Gram-Negative and Gram-positive bacterial families and fungi: Actinomycetales (e.g., Corynebacterium, Mycobacterium, Norcardia), Aspergillosis, Bacillaceae (e.g., Anthrax, Clostridium), Bacteroidaceae, Blastomycosis, Bordetella, Borrelia, Brucellosis, Candidiasis, Campylobacter, Coccidioidomycosis, Cryptococcosis, Dermatocycoses, Enterobacteriaceae (Klebsiella, Salmonella, Serratia, Yersinia), Erysipelothrix, Helicobacter, Legionellosis, Leptospirosis, Listeria, Mycoplasmatales, Neisseriaceae (e.g., Acinetobacter, Gonorrhea, Menigococcal), Pasteurellacea Infections (e.g., Actinobacillus, Heamophilus, Pasteurella), Pseudomonas, Rickettsiaceae, Chlamydiaceae, Syphilis, and Staphylococcal. These bacterial or fungal families can cause the following diseases or symptoms, including, but not limited to: bacteremia, endocarditis, eye infections (conjunctivitis, tuberculosis, uveitis), gingivitis, opportunistic infections (e.g., AIDS related infections), paronychia, prosthesis-related infections, Reiter's Disease, respiratory tract infections, such as Whooping Cough or Empyema, sepsis, Lyme Disease, Cat-Scratch Disease, Dysentery, Paratyphoid Fever, food poisoning, Typhoid, pneumonia, Gonorrhea, meningitis, Chlamydia, Syphilis, Diphtheria, Leprosy, Paratuberculosis, Tuberculosis, Hansen's disease, pulmonary disease resembling tuberculosis, Lymphadenitis, skin disease, disseminated disease, Lupus, Botulism, gangrene, tetanus, impetigo, Rheumatic Fever, Scarlet Fever, sexually transmitted diseases, skin diseases (e.g., cellulitis, dermatocycoses), toxemia, urinary tract infections, wound infections.

Moreover, the methods, modified bacteria, compositions, or vaccine compositions of the present invention can be used to treat or prevent diseases caused by parasitic agents. Those that can be treated by the compounds of the invention include, but are not limited to, the following families: amebiasis, babesiosis, coccidiosis, cryptosporidiosis, dientamoebiasis, dourine, ectoparasitic, giardiasis, helminthiasis, leishmaniasis, theileriasis, toxoplasmosis, trypanosomiasis, and trichomonas.

According to the disclosed methods, modified bacteria, compositions, or vaccine compositions for use in the methods of the present invention can be administered, for example, by intramuscular (i.m.), intravenous (i.v.), subcutaneous (s.c.), or intrapulmonary routes. Other suitable routes of administration include, but are not limited to intratracheal, transdermal, intraocular, intranasal, inhalation, intracavity, intraductal (e.g., into the pancreas), and intraparenchymal (i.e., into any tissue) administration. Transdermal delivery includes, but not limited to intradermal (e.g., into the dermis or epidermis), transdermal (e.g., percutaneous) and transmucosal administration (i.e., into or through skin or mucosal tissue). Intracavity administration includes, but not limited to administration into oral, vaginal, rectal, nasal, peritoneal, or intestinal cavities as well as, intrathecal (i.e., into spinal canal), intraventricular (i.e., into the brain ventricles or the heart ventricles), intraatrial (i.e., into the heart atrium) and sub arachnoid (i.e., into the sub arachnoid spaces of the brain) administration.

Compositions of the present invention further comprise a suitable carrier. Such compositions comprise a therapeutically effective amount of the ceramide-like glycolipid/mycobacteria complex and a pharmaceutically acceptable carrier or excipient. Such a carrier includes but is not limited to saline, buffered saline, dextrose, water, glycerol, ethanol, and combinations thereof. The formulation should suit the mode of administration.

### Pharmaceutical Compositions

The term “pharmaceutically acceptable” refers to compositions that are, within the scope of sound medical judgment, suitable for contact with the tissues of human beings and animals without excessive toxicity or other complications commensurate with a reasonable benefit/risk ratio. In some embodiments, the compositions and vaccines of the present invention are pharmaceutically acceptable.

Ceramide-like glycolipid/bacterial cell complexes of the present invention can be administered in pharmaceutical compositions, e.g., vaccine compositions, in combination with one or more pharmaceutically acceptable excipients, carriers, or dilutents. In certain embodiments, the pharmaceutical compositions, e.g., vaccine compositions of the invention further comprise a heterologous antigen. It will be understood that, when administered to a human patient, the total single or daily usage of the pharmaceutical compositions of the present invention will be decided by the attending physician within the scope of sound medical judgment. The specific therapeutically effective dose level for any particular patient will depend upon a variety of factors including the type and degree of the response to be achieved; the specific composition of another agent, if any, employed; the age, body weight, general health, sex and diet of the patient; the time of administration, route of administration, and rate of excretion of the composition; the duration of the treatment; drugs (such as a chemotherapeutic agent) used in combination or coincidental with the specific composition; and like factors well known in the medical arts. Suitable formulations, known in the art, can be found in Remington's Pharmaceutical Sciences (latest edition), Mack Publishing Company, Easton, Pa.

A composition to be used in a given preventative or therapeutic treatment will be formulated and dosed in a fashion consistent with good medical practice, taking into account the clinical condition of the individual patient (especially the side effects of prevention or treatment with the compounds alone), the site of delivery of the compound, the method of administration, the scheduling of administration, and other factors known to practitioners. The “effective amount” of the compounds of the invention for purposes herein is thus determined by such considerations.

Appropriate dosage of the compositions, e.g., vaccine compositions, of the invention to be administered to a patient will be determined by a clinician. However, as a guide, a suitable amount of a composition of the invention can be between about 10¹ to 10¹² CFU per dose, e.g., 10¹, 10², 10³, 10⁴, 10⁵, 10⁶, 10⁷, 10⁸, 10⁹, 10¹⁰, 10¹¹, or 10¹² CFU, suspended in 0.05 to 0.1 ml of an immunologically inert carrier, e.g., a pharaceutical carrier. In one embodiment, an effective amount of a vaccine of the invention to induce immunity sufficient to prevent or treat, i.e., cure, ameliorate, lessen the severity of or prevent or reduce a diseases described herein is about 10³ to about 10⁷ colony forming units (CFU)/kg body weight. A composition of the invention can be administered as a single dose or multiple doses. The vaccine formulations of the present invention can be employed in dosage forms such as capsules, liquid solutions, suspensions, or elixirs, for oral administration, or sterile liquid for formulations such as solutions or suspensions for, e.g., parenteral, intranasal or topical administration.

Compositions of the invention can be administered orally, intravenously, rectally, parenterally, intracisternally, intradermally, intravaginally, intraperitoneally, topically (as by powders, ointments, gels, creams, drops or transdermal patch), bucally, or as an oral or nasal spray. The term “parenteral” as used herein refers to modes of administration which include intravenous, intramuscular, intraperitoneal, intrasternal, subcutaneous and intraarticular injection and infusion.

Compositions, e.g, vaccine compositions, of the invention can be formulated according to known methods. Suitable preparation methods are described, for example, in Remington's Pharmaceutical Sciences, 16th Edition, A. Osol, ed., Mack Publishing Co., Easton, Pa. (1980), and Remington's Pharmaceutical Sciences, 19th Edition, A. R. Gennaro, ed., Mack Publishing Co., Easton, Pa. (1995), both of which are incorporated herein by reference in their entireties. Although the composition can be administered as an aqueous solution, it can also be formulated as an emulsion, gel, solution, suspension, lyophilized form, or any other form known in the art. In addition, the composition can contain pharmaceutically acceptable additives including, for example, diluents, binders, stabilizers, and preservatives. Once formulated, the compositions of the invention can be administered directly to the subject. The subjects to be treated can be animals; in particular, human subjects can be treated.

In certain embodiments, a host cell, e.g., a bacterial cell, having a vector expressing a polypeptide, e.g., an immunogenic polypeptide, of the present invention is incorporated in a composition. The concentration of polypeptides of the invention in the compositions of the invention can vary widely, i.e., from less than about 0.1%, usually at or at least about 2% to as much as 20% to 50% or more by weight, and will be selected primarily by fluid volumes, viscosities, etc., in accordance with the particular mode of administration selected.

Compositions of the invention ordinarily will be stored in unit or multi-dose containers, for example, sealed ampules or vials, as an aqueous solution or as a lyophilized formulation for reconstitution. Mycobacterial compositions with directly incorporated glycolipid adjuvant can be lypohilized and the adjuvant activity will be recovered intact when the composition is rehydrated and suspended for injection. As an example of a lyophilized formulation, 10-ml vials are filled with 5 ml of sterile-filtered 1% (w/v) aqueous solution, and the resulting mixture is lyophilized. An infusion solution is prepared by reconstituting the lyophilized composition using water, e.g., bacteriostatic Water-for-Injection.

Compositions of the invention are useful for administration to any animal, for example a mammal (such as apes, cows, horses, pigs, boars, sheep, rodents, goats, dogs, cats, chickens, monkeys, rabbits, ferrets, whales, and dolphins), and a human.

Animal models that have been shown to be good correlates for human disease include, but are not limited to guinea pigs and non-human primates (See e.g., Balasubramanian V et al., Immunobiology 191(4-5):395-401 (1994) and Barclay W R et al., Infect. Immun. 2(5):574-582 (1970), both incorporated herein by reference in their entirety).

The invention also provides a pharmaceutical pack or kit comprising one or more containers filled with one or more of the ingredients of the pharmaceutical compositions of the invention. Associated with such containers can be a notice in the form prescribed by a governmental agency regulating the manufacture, use or sale of pharmaceuticals or biological products, which notice reflects approval by the agency of manufacture, use or sale for human administration. In addition, the compositions of the present invention can be employed in conjunction with other therapeutic compositions.

Suitable preparations of such vaccines include, but are not limited to injectables, either as liquid solutions or suspensions; solid forms suitable for solution in, or suspension in liquid prior to injection, can also be prepared. The preparation can also be emulsified, or the polypeptides encapsulated in liposomes. The active immunogenic ingredients are often mixed with excipients which are pharmaceutically acceptable and compatible with the active ingredient. Suitable excipients are, for example, water, saline, dextrose, glycerol, or the like and combinations thereof. In addition, if desired, the vaccine preparation can also include minor amounts of auxiliary substances such as wetting or emulsifying agents, pH buffering agents, and/or adjuvants which enhance the effectiveness of the vaccine.

Compositions of the present invention which comprise, a ceramide-like glycolipid/bacterial cell complex can further comprise additional adjuvants. Examples of adjuvants which can be effective are described above and can include, but are not limited to: aluminum hydroxide, N-acetyl-muramyl-L-threonyl-D-isoglutamine (thr-MDP), N-acetyl-nor-muramyl-L-alanyl-D-isoglutamine, N-acetylmuramyl-L-alanyl-D-isoglutaminyl-L-alanine-2-(1′-2′-dipalmitoyl-sn-glycero-3-hydroxyphosphoryloxy)-ethylamine, GM-CSF, QS-21 (investigational drug, Progenies Pharmaceuticals, Inc.), DETOX (investigational drug, Ribi Pharmaceuticals), BCG, and CpG rich oligonucleotides.

Compositions of the present invention which comprise a ceramide-like glycolipid/bacterial cell complex can further comprise additional adjuvants which are also Toll-like receptor (TLR) agonists. Examples of TLR agonist adjuvants which can be effective, include, but are not limited to: N-acetylmuramyl-L-alanine-D-isoglutamine (MDP), lipopolysaccharides (LPS), genetically modified and/or degraded LPS, alum, glucan, colony stimulating factors (e.g., EPO, GM-CSF, G-CSF, M-CSF, PEGylated G-CSF, SCF, IL-3, IL6, PIXY 321), interferons (e.g., γ-interferon, α-interferon), interleukins (e.g., IL-2, IL-7, IL-12, IL-15, IL-18), saponins (e.g., QS21), monophosphoryl lipid A (MPL), 3 De-O-acylated monophosphoryl lipid A (3D-MPL), unmethylated CpG sequences, 1-methyl tryptophan, arginase inhibitors, cyclophosphamide, antibodies that block immunosuppressive functions (e.g., anti-CTLA4 antibodies), lipids (such as palmitic acid residues), tripalmitoyl-S-glycerylcystein lyseryl-serine (P₃ CSS), and Freund's adjuvant. Other adjuvant examples include compounds such as isatoribin and it derivatives (Anadys Pharmaceuticals) or imidazoquinolinamines, such as imiquimod and resiquimod (Dockrell & Kinghom, J. Antimicrob. Chemother., 48:751-755 (2001) and Hemmi et al., Nat. Immunol., 3:196-200 (2002), guanine ribonucleosides, such as C8-substituted or N7, C-8-disubstituted guanine ribonucleosides (Lee et al., Proc. Natl. Acad. Sci. USA, 100:6646-6651 (2003) and the compounds that are disclosed in Pat. Pub. Nos. JP-2005-089,334; WO99/32122; WO98/01448 WO05/092893; and WO05/092892, and TLR-7 agonist SM360320 (9-benzyl-8-hydroxy-2-(2-methoxy-ethoxy)aderine) disclosed in Lee et al., Proc Natl Acad Sci USA, 103(6):1828-1833 (2006).

In addition to isatoribin, other TLR agonist adjuvants include 9-benzyl-8-hydroxy-2-(2-methoxyethoxy)adenine (SM360320), Actilon™ (Coley Pharmaceutical Group, Inc.), and the following compounds by Sumitmo Pharmaceutical Co, Ltd.:

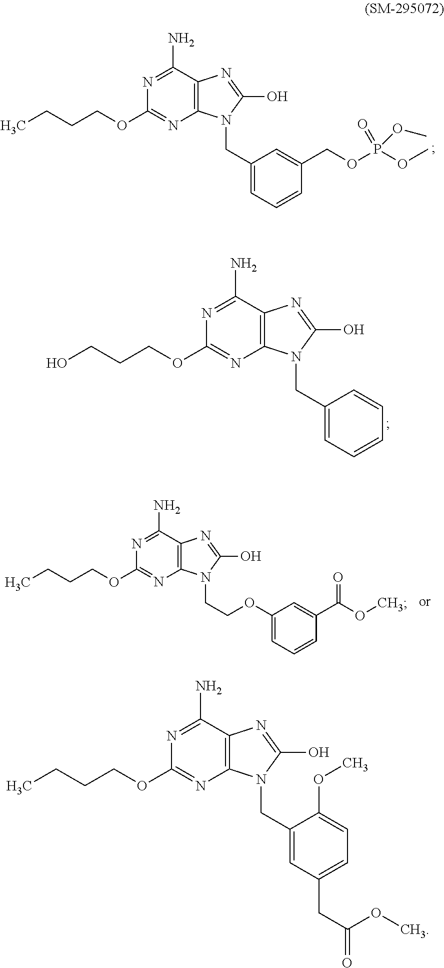

Other adjuvants which can be used in conjunction with the composition of the present invention are disclosed in PCT Pub. No. WO 2005/000348, U.S. Pat. Pub. No. 2007/0292418, and U.S. Pat. Pub. No. 2007/0287664.

The composition, if desired, can also contain minor amounts of wetting or emulsifying agents, or pH buffering agents. The composition can be a liquid solution, suspension, emulsion, tablet, pill, capsule, sustained release formulation, or powder. Oral formulation can include standard carriers such as pharmaceutical grades of mannitol, lactose, starch, magnesium stearate, sodium saccharine, cellulose, magnesium carbonate, etc.

The compositions of the present invention can further comprise other compounds which modulate an immune response, for example, cytokines. The term “cytokine” refers to polypeptides, including, but not limited to, interleukins (e.g., IL-1, IL-2, IL-3, IL-4, IL-5, IL-6, IL-7, IL-8, IL-9, IL-10. IL-11, IL-12, IL-13, IL-14, IL-15, IL-16, IL-17, and IL-18), α interferons (e.g., IFN-α), β interferon (IFN-β), γ interferons (e.g., IFN-γ), colony stimulating factors (CSFs, e.g., CSF-1. CSF-2, and CSF-3), granulocyte-macrophage colony stimulating factor (GMCSF), transforming growth factor (TGF, e.g., TGFα and TGFβ), and insulin-like growth factors (IGFs, e.g. IGF-I and IGF-II).

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See. for example, Molecular Cloning A Laboratory Manual, 2nd Ed., Sambrook et al., ed., Cold Spring Harbor Laboratory Press: (1989); Molecular Cloning: A Laboratory Manual, Sambrook et al., ed., Cold Springs Harbor Laboratory, New York (1992), DNA Cloning, D. N. Glover ed., Volumes I and II (1985); Oligonucleotide Synthesis, M. J. Gait ed., (1984); Mullis et al. U.S. Pat. No. 4,683,195; Nucleic Acid Hybridization, B. D. Hames & S. J. Higgins eds. (1984); Transcription And Translation, B. D. Hames & S. J. Higgins eds. (1984), Culture Of Animal Cells, R. I. Freshney, Alan R. Liss, Inc., (1987); Immobilized Cells And Enzymes, IRL Press, (1986); B. Perbal, A Practical Guide To Molecular Cloning (1984); the treatise, Methods In Enzymology, Academic Press, Inc., N.Y.; Gene Transfer Vectors For Mammalian Cells, J. H. Miller and M. P. Calos eds., Cold Spring Harbor Laboratory (1987); Methods In Enzymology, Vols. 154 and 155 (Wu et al. eds.); Immunochemical Methods In Cell And Molecular Biology, Mayer and Walker, eds., Academic Press, London (1987); Handbook Of Experimental Immunology, Volumes I-IV, D. M. Weir and C. C. Blackwell, eds., (1986); Manipulating the Mouse Embryo, Cold Spring Harbor Laboratory Press, Cold Spring Harbor, N.Y., (1986); and in Ausuhel et al., Current Protocols in Molecular Biology, John Wiley and Sons, Baltimore, Md. (1989).

General principles of antibody engineering are set forth in Antibody Engineering, 2nd edition, C.A.K. Borrebaeck, Ed., Oxford Univ. Press (1995). General principles of protein engineering are set forth in Protein Engineering, A Practical Approach, Rickwood, D., et al., Eds., IRL Press at Oxford Univ. Press, Oxford, Eng. (1995). General principles of antibodies and antibody-hapten binding are set forth in: Nisonoff, A., Molecular Immunology, 2nd ed., Sinauer Associates, Sunderland, Mass. (1984); and Steward, M. W., Antibodies, Their Structure and Function, Chapman and Hall, New York, N.Y. (1984). Additionally, standard methods in immunology known in the art and not specifically described are generally followed as in Current Protocols in Immunology, John Wiley & Sons, New York; Stites et al. (eds), Basic and Clinical-Immunology (8th ed.), Appleton & Lange, Norwalk, Conn. (1994) and Mishell and Shiigi (eds), Selected Methods in Cellular Immunology, W.H. Freeman and Co., New York (1980).

Standard reference works setting forth general principles of immunology include Current Protocols in Immunology, John Wiley & Sons, New York; Klein, J., Immunology: The Science of Self-Nonself Discrimination, John Wiley & Sons, New York (1982); Kennett, R., et al., eds., Monoclonal Antibodies, Hybridoma: A New Dimension in Biological Analyses, Plenum Press, New York (1980); Campbell, A., “Monoclonal Antibody Technology” in Burden, R., et al., eds., Laboratory Techniques in Biochemistry and Molecular Biology, Vol. 13, Elsevere, Amsterdam (1984), Kuby Immunnology 4th ed. Ed. Richard A. Goldsby, Thomas J. Kindt and Barbara A. Osborne, H. Freemand & Co. (2000); Roitt, I., Brostoff, J. and Male D., Immunology 6th ed. London: Mosby (2001); Abbas A., Abul, A. and Lichtman, A., Cellular and Molecular Immunology Ed. 5, Elsevier Health Sciences Division (2005); Kontermann and Dubel, Antibody Engineering, Springer Verlan (2001); Sambrook and Russell, Molecular Cloning: A Laboratory Manual. Cold Spring Harbor Press (2001); Lewin, Genes VIII, Prentice Hall (2003); Harlow and Lane, Antibodies: A Laboratory Manual, Cold Spring Harbor Press (1988); Dieffenbach and Dveksler, PCR Primer Cold Spring Harbor Press (2003).

The entire disclosure, including the materials and methods, disclosed in PCT/US2010/020521, filed Jan. 8, 2010, is hereby incorporated by reference in its entirety.

All of the references cited above, as well as all references cited herein, are incorporated herein by reference in their entireties.

### EXAMPLES

### Example 1

### Stable Enhancement of Recombinant BCG Vaccines with iNKT Cell Activators

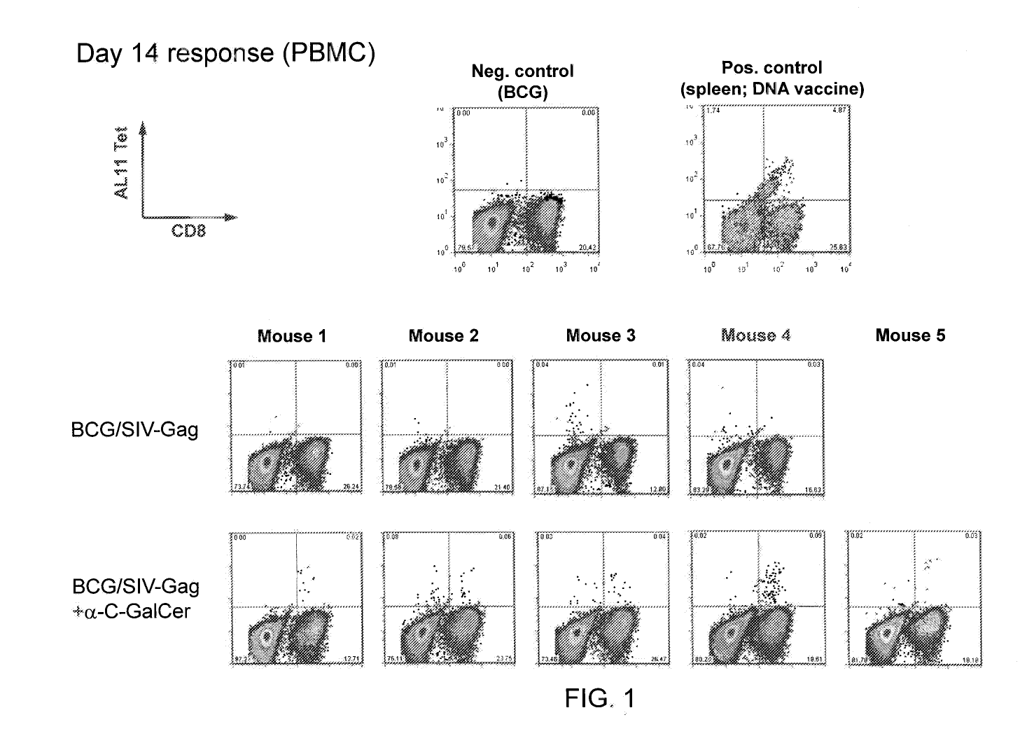

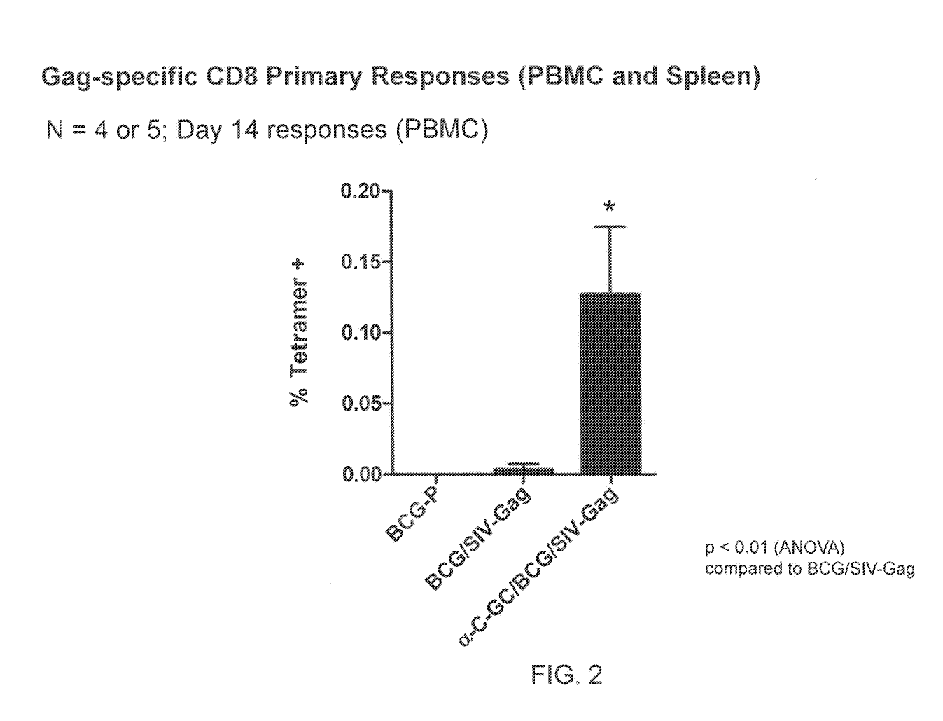

The peripheral blood mononuclear cell (PBMC) primary response to BCG/SIV-Gag with and without of incorporation of an iNKT cell activating glycolipid (α-C-GalCer) was tested. Simian Immunodeficiency Virus (SIV)-Gag is a BCG-Pasteur strain expressing SIV-Gag protein (an heterologous antigen). The BCG/SIV-Gag cells comprise a full-length SIV Mac239 Gag (codon optimized), Hsp60 promoter, 19 kDa LP ss, non-integrating multicopy pasmid (Cayabyab et al., J. Virol. 83(11):5505-5513 (2009)). C57BL/6 mice were immunized with BCG (BCG-P); BCG/SIV-Gag; or α-C-GalCer modified BCG/SIV-Gag (10⁷ CFU, i.v.). FIG. 1 shows the primary response (PBMC) at day 14. The Gag-specific CD8+ T cells were quantitated in PBMC at day 14 using AL11 tetramer staining (H-2 Db/Gag specific). FIG. 2 shows an increase in Gag-specific CD8+ T primary response in mice immunized with α-C-GalCer modified BCG/SIV-Gag compared to BCG alone or BCG/SIV-Gag.

These results show that incorporation of an iNKT cell activating glycolipid (α-C-GalCer) into a BCG strain expressing a heterologous antigen (SIV-Gag protein) improves the function of the bacterium as an antigen carrier for priming immune responses against the heterologous antigen.

### Example 2

### Recombinant Ad5/SIV-Gag Boosting of Mice Primed with BCG/SIV-Gag with or without αGalCer Modification

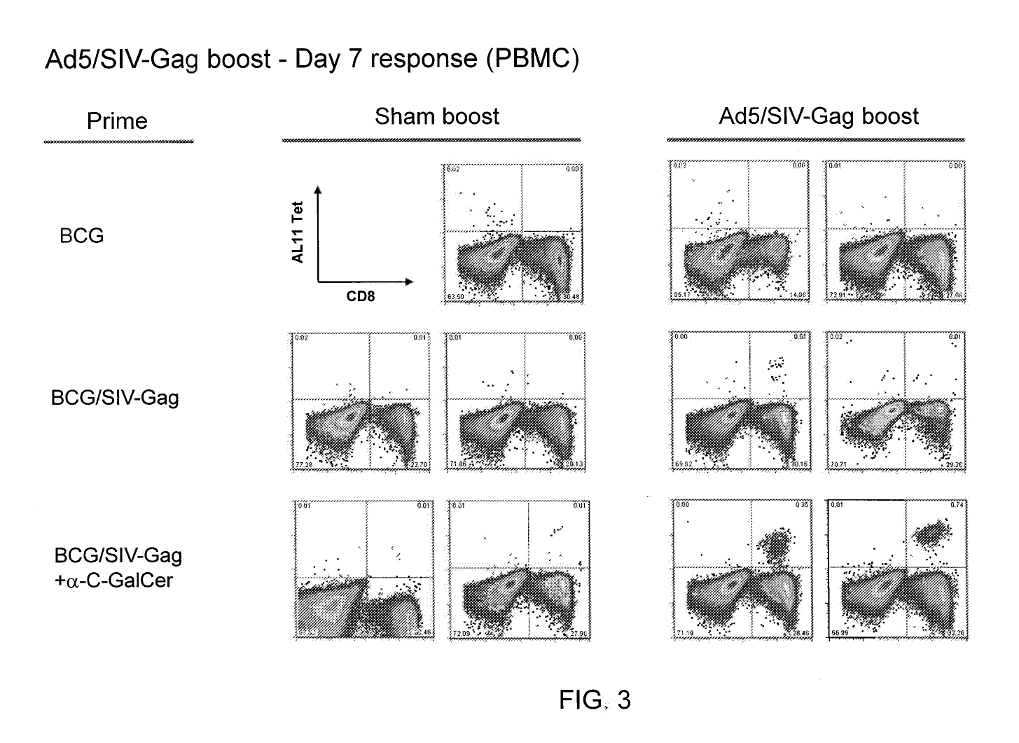

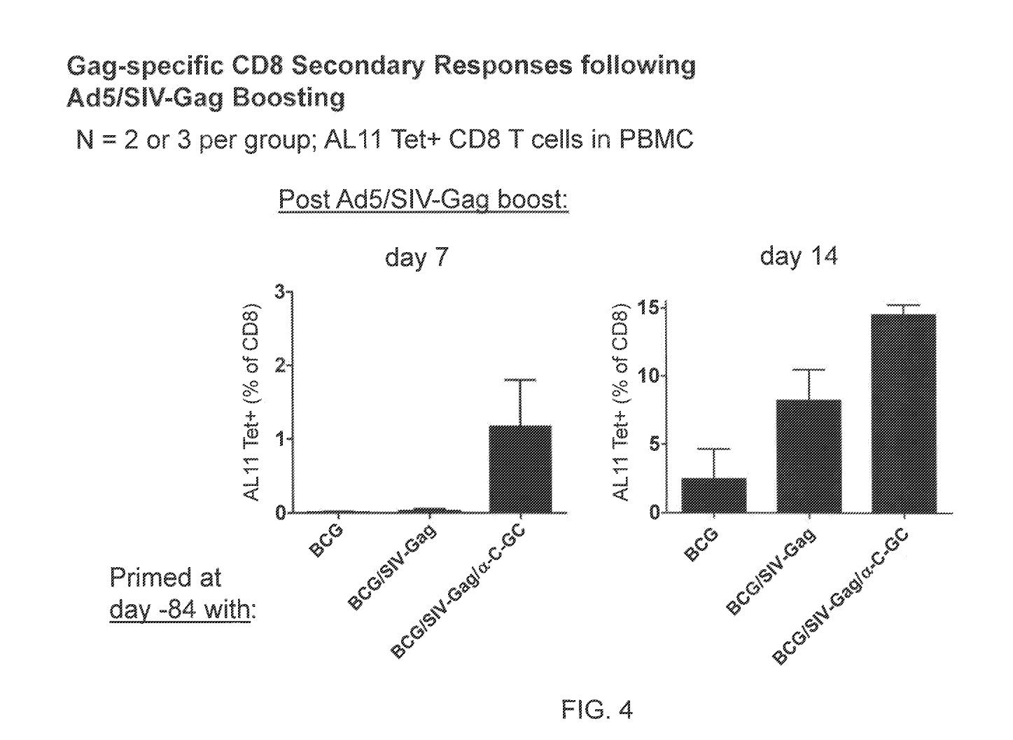

The effect of α-C-GalCer incorporation on peripheral blood mononuclear cell (PBMC) response to GCG/SIV-Gag in mice given a subsequent boost with rAd5/SIV-Gag was tested. Replication defective Adenovirus (serotype 5) expressing full length SIV Mac239 Gag (rAd5/SIV-Gag, GenVec) was used for the subsequent boosting. C57BL/6 mice were primed with BCG; BCG/SIV-Gag; or α-C-GalCer modified BCG/SIV-Gag (10⁷ CFU, retro-orbital). Twelve weeks later, the mice were administered with a suboptimal dose of rAd5/SIV-Gag (10⁷ PFU, i.m.), or sham boost with saline. FIG. 3 shows the secondary response (PBMC) at day 7. The CD8+ T cell response by AL11 tetramer staining of PBMC was assessed at day 7 and day 14 post-boosting. FIG. 4 shows an increase in Gag-specific CD8+ T secondary response in mice immunized with α-C-GalCer modified BCG/SIV-Gag compared to BCG alone or BCG/SIV-Gag.

These results show that enhanced priming with α-C-GalCer modified BCG/SIV-Gag also translated into enhanced boosting of the secondary response. These results are relevant to using the glycolipid modified mycobacteria as antigen carriers for delivery of antigens against infectious agents or tumors.

### Example 3

### An Improved Method for Incorporation of Glycolipids into Live Mycobacteria

A method for incorporating an exemplary ceramide-like glycolipid, αGalCer, into the cell wall of a mycobacterium was tested. The method involved coupling protecting groups to the hydroxyls to make the glycolipid more apolar and therefore soluble in petroleum ether (PetEther). Live mycobacteria were suspended in the hydroxyl-protected glycolipid solvent solution, and the solvent was then evaporated.

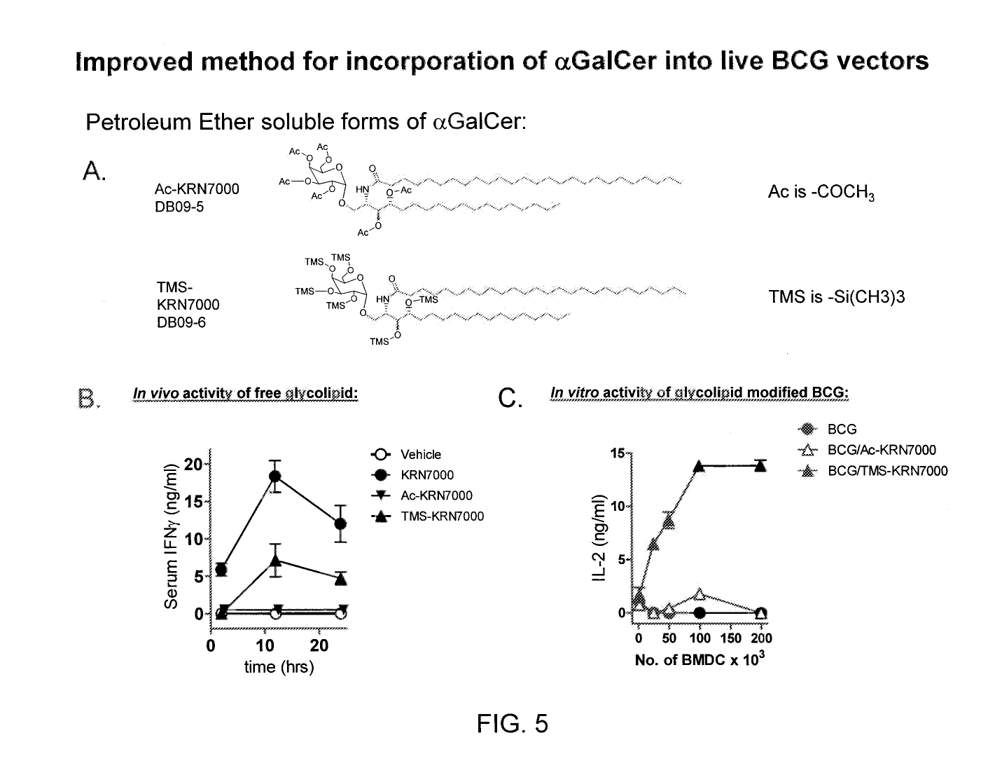

Protecting groups (acetyl and TMS) were coupled to hydroxyls of an αGalCer glycolipid ((2S,3S,4R)-1-O-(α-D-galactopyranosyl)-N-hexacosanoyl-2-amino-1,3,4-octadecanetriol (KRN7000)). The structures of Ac-KRN700 DB09-5 and TMS-KRN700 DB09-6 are shown in FIG. 5A. For the TMS coupling procedure, a mixture of pyridine (5 mL) and hexamethyldisilazane (1 mL), and chlorotrimethylsilane (0.5 mL) was added to a solution of αGalCer (10 mg). The resulting mixture was then stirred at 70° C. for 1 hour under an argon atmosphere and then allowed to cool to room temperature. The reaction mixture was then poured into ice water (10 mL) and extracted with hexanes (3×10 mL). The organic extracts were combined, dried over anhydrous sodium sulphate, and concentrated under vacuo. The resulting residue was purified by flash column chromatography hexanes/EtOAc (10:1) to afford the per-TMS protected αGalCer (TMS-KRN7000). Standard procedures were used for the acetylation coupling reaction to prepare the Ac-KRN7000.

The evaporation was done by directing a stream of nitrogen (or argon) gas onto the surface of the liquid in an open container at room temperature (˜22° C.). The hydroxyl-protected αGalCer molecules (Ac-KRN7000, and TMS-KRN7000) were were found to be soluble in solvent (PetEther).

The in vivo activity of free glycolipid (KRN7000, Ac-KRN7000, and TMS-KRN7000) compared to vehicle control was tested. Activation of mouse splenocytes with KRN7000- and TMS-KRN7000-infected BMDCs induced IFNγ production (FIG. 5B).

The mycobacterial cell, BCG, is a live attenuated bacterial vaccine which is actively ingested by APCs and processed for antigen presentation. Live BCG were suspended in the PetEther solution comprising the hydroxyl-protected αGalCer, and thereafter the solvent was evaporated. This method resulted in the αGalCer being transferred into the mycobacteria cell wall. Both TMS and Ac were soluble in PetEther, and resulted in hydroxyl protected αGalCer being incorporated into BCG using PetEther solvent. Thus, the described method resulted in ceramide-like glycolipid, αGalCer, being incorporated into a mycobacterial cell wall allowing for simultaneous administration of both the glycolipid adjuvant and the BCG vaccine.

Alpha-GalCer bound to the BCG cell wall using the method described herein was biologically active in vitro. Alpha-GalCer and its analogues are known to activate NKT cells in vitro. This biological activity was tested to determine whether ceramide-like glycolipids incorporated into a mycobacterial cell wall using the described method retained the ability to activate NKT cells in vitro. Bone Marrow-derived Dendritic Cells (BMDC) infected with BCG, BCG/Ac-KRN7000, and BCG/TMS-KRN7000 were incubated with an NKT cell hybridoma. For BCG/TMS-KRN7000, IL-2 was detectable in the supernatant in a dose-dependant manner indicating very efficient stimulation of iNKT cells in vitro by the ceramide-like glycolipids which were bound to the BCG cell wall (FIG. 5C). Thus, the αGalCer activity was retained using the described method. It is noted that the protecting groups might be removed (deprotected) within the cell after the ceramide-like glycolipid is incorporated into the mycobacterial cell wall. These results show that non-native glycolipids can be incorporated into a mycobacterial cell wall using the methods described herein.

The entire disclosure of all publications (including patents, patent applications, journal articles, laboratory manuals, books, or other documents) cited herein are hereby incorporated by reference.

1. A modified bacterium comprising: a bacterial cell, an heterologous antigen, and a ceramide-like glycolipid, wherein said ceramide-like glycolipid is physically associated with said bacterial cell.

2. The modified bacterium of claim 1, wherein said ceramide-like glycolipid comprises a glycosylceramide or an analog thereof.

3. The modified bacterium of claim 2, wherein said glycosylceramide or analog thereof comprises Formula I: 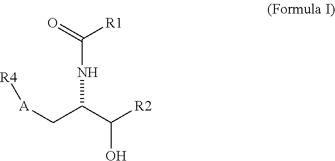
wherein R1 is a linear or branched C₁-C₂₇ alkane or C₂-C₂₇ alkene; or R1 is —C(OH)—R3 wherein R3 is a linear or branched C₁-C₂₆ alkane or C₂-C₂₆ alkene; or R1 is a C₆-C₂₇ alkane or alkene wherein (i) the C₆-C₂₇ alkane or alkene is substituted with a C₅-C₁₅ cycloalkane, C₅-C₁₅ cycloalkene, heterocycle, or aromatic ring or (ii) the C₆-C₂₇ alkane or alkene includes, within the C₆-C₂₇ alkyl or alkenyl chain, a C₅-C₁₅ cycloalkane, C₅-C₁₅ cycloalkene, heterocycle, or aromatic ring;
R2 is one of the following (a)-(e):
—CH₂(CH₂)ₓCH₃, (a)
—CH(OH)(CH₂)ₓCH₃, (b)
—CH(OH)(CH₂)ₓCH(CH₃)₂, (c)
—CH═CH(CH₂)ₓCH₃, (d)
—CH(OH)(CH₂)ₓCH(CH₃)CH₂CH₃, (e)
wherein X is an integer ranging from 4-17;
R4 is an α-linked or a β-linked monosaccharide, or when R1 is a linear or branched C₁-C₂₇ alkane, R4 is: 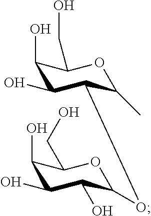
and
A is O or —CH₂.

4. The modified bacterium of claim 3, wherein R1 is —(CH₂)₂₂CH₃ or —(CH₂)₂₄—CH₃; wherein R2 is —CH(OH)—(CH₂)₁₃CH₃; or wherein R4 is galactosyl, mannosyl, fucosyl or glucosyl.

5-6. (canceled)

7. The modified bacterium of claim 1, wherein said ceramide-like glycolipid comprises an α-galactosylceramide or an analog thereof.

8. The modified bacterium of claim 7, wherein said α-galactosylceramide or analog thereof comprises Formula II: 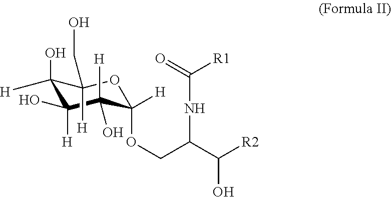
wherein
R1 is a linear or branched C₁-C₂₇ alkane or C₂-C₂₇ alkene; or R1 is —C(OH)—R3 wherein R3 is linear or branched C₁-C₂₆ alkane or C₂-C₂₆ alkene; and
R2 is one of the following (a)-(e):
—CH₂(CH₂)ₓCH₃, (a)
—CH(OH)(CH₂)ₓCH₃, (b)
—CH(OH)(CH₂)ₓCH(CH₃)₂, (c)
—CH═CH(CH₂)ₓCH₃, (d)
—CH(OH)(CH₂)ₓCH(CH₃)CH₂CH₃, (e)
wherein X is an integer ranging from 4-17.

9. The modified bacterium of claim 8, wherein R2 is —CH(OH)(CH₂)ₓCH₃, wherein X is an integer ranging from 4-13.

10. The modified bacterium of claim 8, wherein R1 is selected from the group consisting of (CH₂)₉CH═CH—CH₂—CH═CH(CH₂)₄CH₃, (CH₂)₈CH═CH—CH₂—CH═CH(CH₂)₄CH₃, (CH₂)₇CH═CH—CH₂—CH═CH(CH₂)₄CH₃, (CH₂)₃CH═CH—CH₂—CH═CH—CH₂—CH═CH—CH₂—CH═CH—(CH₂)₄CH₃, (CH₂)₃CH═CH—CH₂—CH═CH—CH₂—CH═CH—CH₂—CH═CH—CH₂—CH═CH—CH₂CH₃, (CH₂)₇CH═CH—CH₂—CH═CH═(CH₂)₄CH₃, (CH₂)₇CH═CH—CH═CH(CH₂)₅CH₃, (CH₂)₈CH═CH—CH═CH(CH₂)₄CH₃, (CH₂)₉CH═CH—CH═CH(CH₂)₅CH₃, (CH₂)₆CH═CH—CH═CH—CH═CH(CH₂)₄CH₃, (CH₂)₆CH═CH—CH═CH—CH═CH(CH₂)₄CH₃ and (CH₂)₇CH═CH—CH═CH—CH═CH(CH₂)₃CH₃.

11. The modified bacterium of claim 10, wherein the double bonds are cis or trans.

12. The modified bacterium of claim 7, wherein said α-galactosylceramide or analog thereof comprises Formula III: 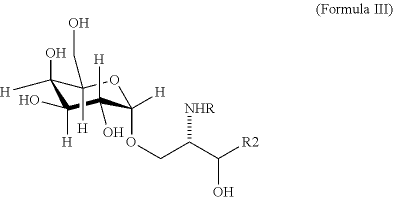
wherein R is H or —C(O)R1, wherein R1 is a linear or branched C₁-C₂₇ alkane or C₂-C₂₇ alkene; or R1 is —C(OH)—R3 wherein R3 is a linear or branched C₁-C₂₆ alkane or C₂-C₂₆ alkene; or R1 is a C₆-C₂₇ alkane or alkene wherein (i) the C₆-C₂₇ alkane or alkene is substituted with a C₅-C₁₅ cycloalkane, C₅-C₁₅ cycloalkene, heterocycle, or aromatic ring or (ii) the C₆-C₂₇ alkane or alkene includes, within the C₆-C₂₇ alkyl or alkenyl chain, a C₅-C₁₅ cycloalkane, C₅-C₁₅ cycloalkene, heterocycle, or aromatic ring; or R1 is a —(CH₂)ₙR5, wherein n is an integer ranging from 0-5, and R5 is —C(O)OC₂H₅, an optionally substituted C₅-C₁₅ cycloalkane, an optionally substituted aromatic ring, or an aralkyl, and
R2 is one of the following (a)-(e):
—CH₂(CH₂)ₓCH₃, (a)
—CH(OH)(CH₂)ₓCH₃, (b)
—CH(OH)(CH₂)ₓCH(CH₃)₂, (c)
—CH═CH(CH₂)ₓCH₃, (d)
—CH(OH)(CH₂)ₓCH(CH₃)CH₂CH₃, (e)
wherein X is an integer ranging from 4-17.

13. The modified bacterium of claim 12, wherein R1 is substituted with oxo; hydroxy; halogen; phenyl; —OC(O)R6; —OR6; —C(O)R6; or N(R6)₂,
wherein each R6 is independently hydrogen, C₁-C₆ alkyl, or an aromatic ring optionally substituted with halogen; hydroxy; —OC(O)R7; —OR7; —C(O)R7 or N(R7)₂, and
wherein each R7 is independently hydrogen or C₁-C₆ alkyl; or wherein R1 is selected from the group consisting of 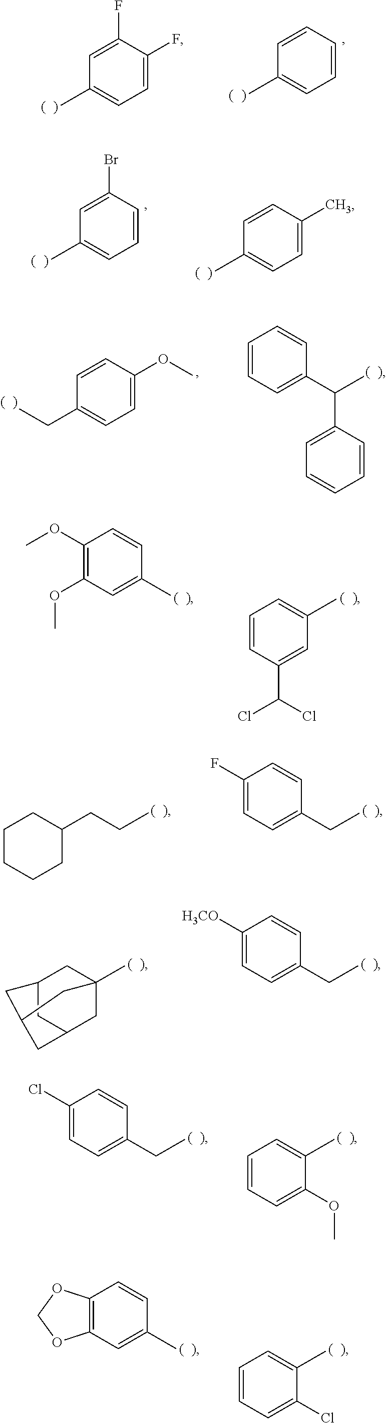 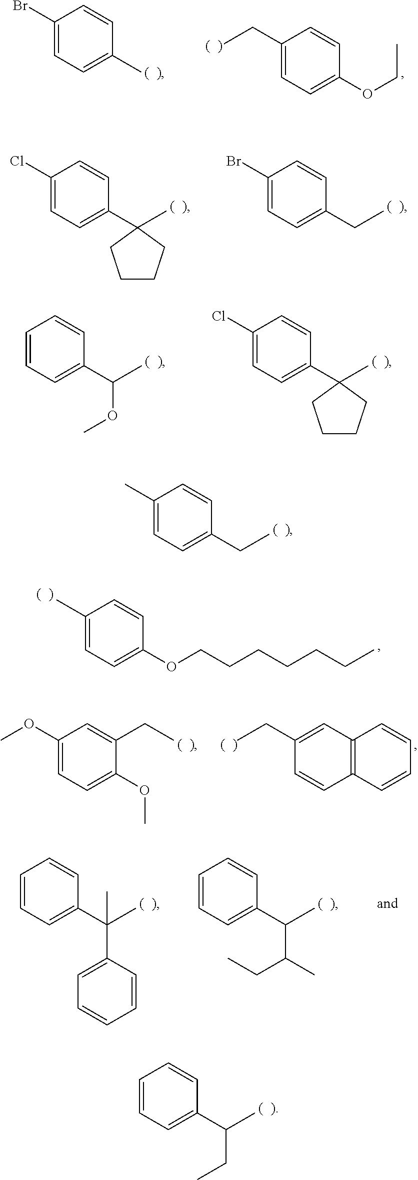
where ( ) represent the point of attachment of R1 to the compound of Formula III.

14. (canceled)

15. The modified bacterium of claim 7, wherein said α-galactosylceramide or analog thereof comprises (2S,3S,4R)-1-O-(α-D-galactopyranosyl)-N-hexacosanoyl-2-amino-1,3,4-octadecanetriol (KRN7000), (2S,3S)-1-O-(α-D-galactopyranosyl)-N-hexacosanoyl-2-amino-1,3-octadecanediol), or (2S,3S,4R)-1-CH₂-(α-galactopyranosyl)-N-hexacosanoyl-2-amino-1,3,4-octadecanetriol (α-C-GalCer).

16. (canceled)

17. The modified bacterium of claim 1, wherein said ceramide-like glycolipid is incorporated into the cell wall of said bacterial cell.

18. The modified bacterium of claim 1, wherein said bacterial cell is selected from the group consisting of a mycobacterial cell, a Listeria cell, a Salmonella cell, a Yersinia cell, a Francisella cell, and a Legionella cell.

19. (canceled)

20. The modified bacterium of claim 19, wherein said mycobacterial cell is selected from the group consisting of a M. tuberculosis complex (MTBC) cell and a nontuberculous mycobacterial (NTM) cell.

21. (canceled)

22. The modified bacterium of claim 21, wherein said MTBC cell is selected from the group consisting of a M. tuberculosis cell, a M. bovis cell, a M. bovis bacille Calmette-Guérin (BCG) cell, a M. africanum cell, a M. canetti cell, a M. caprae cell, and a M. pinnipedii' cell.

23-25. (canceled)

26. The modified bacterium of claim 20, wherein said NTM cell is a M. smegmatis cell.

27. The modified bacterium of claim 1,
wherein said bacterial cell is live, killed, or attenuated.

28. The modified bacterium of claim 1, which enhances antigen-specific CD8 T cell responses against the heterologous antigen.

29. The modified bacterium of claim 1, wherein said heterologous antigen is expressed on the surface of said bacterial cell.

30. The modified bacterium of claim 1, wherein said heterologous antigen is a viral antigen, a bacterial antigen, a fungal antigen, a parasitic antigen, or a tumor specific antigen.

31. (canceled)

32. The modified bacterium of claim 29, wherein said heterologous antigen is an immunogenic peptide.

33-34. (canceled)

35. A composition comprising the modified bacterium of claim 1, and a pharmaceutical carrier.

36. (canceled)

37. The composition of claim 35, further comprising an adjuvant.

38. (canceled)

39. A method of treating or preventing a disease in an animal, comprising administering to an animal in need of said treatment or prevention the modified bacterium of claim 1; wherein said modified bacterium is administered in an amount sufficient to alter the progression of said disease.

40. The method of claim 39, wherein an immune response is enhanced or modified relative to an immune response produced by the bacterial cell not associated with said ceramide-like glycolipid.

41. The method of claim 40, wherein said immune response is a priming immune response against the heterologous antigen.

42. The method of claim 39, wherein said disease is selected from the group consisting of a viral disease, a bacterial disease, a fungal disease, a parasitic disease, and a proliferative disease.

43. The method of claim 39, wherein said disease is selected from the group consisting of tuberculosis, pulmonary disease resembling tuberculosis, lymphadenitis, skin disease, disseminated disease, bubonic plague, pneumonic plague, tularemia, Legionairre's disease, anthrax, typhoid fever, paratyphoid fever, foodborne illness, listeriosis, malaria, HIV, SIV, HPV, RSV, influenza, hepatitis (HAV, HBV, and HCV), and cancer.

44. The method of claim 39, further comprising administration of a booster vaccine comprising the heterologous antigen following the administration of the modified bacterium of claim 1.

45. The method of claim 44, wherein said booster vaccine is a recombinant adenovirus.

46. A method of inducing an immune response against an antigen in an animal, comprising administering to said animal the modified bacterium of claim 1.

47. The method of claim 46, wherein said modified bacterium is administered in an amount sufficient to enhance an antigen-specific CD8 T-cell response or enhance the activity of Natural Killer T (NKT) cells in said animal.

48. The method of claim 47, wherein said immune response comprises an antibody response, a CD8 T-cell response, or a combination thereof.

49-50. (canceled)

51. A method of modulating a CD8 T-cell response to BCG in an animal comprising administering to said animal an effective amount of the modified bacterium of claim 1, wherein said bacterial cell is a BCG cell.

52. The method of claim 39, wherein said administration is by a route selected from the group consisting of intramuscularly, intravenously, intratracheally, intranasally, transdermally, intradermally, subcutaneously, intraocularly, vaginally, rectally, intraperitoneally, intraintestinally, by inhalation, or by a combination of two or more of said routes.

53. A kit comprising: the modified bacterium of claim 1.

54. The kit of claim 53, wherein said modified bacterium is lyophilized.

55. The kit of claim 53, further comprising a means for administering said modified bacterium.

56. A method of making a ceramide-like glycolipid/mycobacterial complex comprising (a) coupling a protection group to the hydroxyls of a ceramide-like glycolipid; (b) adding a solvent to the hydroxyl-protected ceramide-like glycolipid of (a) to produce a hydroxyl-protected ceramide-like glycolipid solvent solution; (c) suspending a mycobacterium in the hydroxyl-protected ceramide-like glycolipid solvent solution; and (d) evaporating said solvent, thereby making said ceramide-like glycolipid/mycobacterial complex.

57. The method claim 56, wherein said solvent is a nonpolar solvent.

58. The method of claim 57, wherein the solvent is petroleum ether.

59. The method of claim 56, wherein the protection group is an alcohol protecting group.

60. The method of claim 59, wherein said alcohol protecting group is a Silyl ether.

61. The method of claim 60, wherein the Silyl ether is selected from the group consisting of trimethylsilyl (TMS), tert-butyldimethylsilyl (TBDMS), tert-butyldimethylsilyloxymethyl (TOM), and triisopropylsilyl (TIPS) ether.

62. (canceled)

63. The method of claim 56, wherein said mycobacterial cell is selected from the group consisting of a M. tuberculosis complex (MTBC) cell and a nontuberculous mycobacterial (NTM) cell.

64. The method of claim 63, wherein said MTBC cell is selected from the group consisting of a M. tuberculosis cell, a M. bovis cell, a BCG cell, a M. africanum cell, a M. canetti cell, a M. caprae cell, and a M. pinnipedii' cell.

65. (canceled)

66. The method of claim 56, wherein said mycobacterial cell is live, killed, or attenuated.

67. The method of claim 56, wherein said ceramide-like glycolipid comprises a glycosylceramide or an analog thereof.

68. The method of claim 67, wherein said glycosylceramide or analog thereof comprises Formula I: 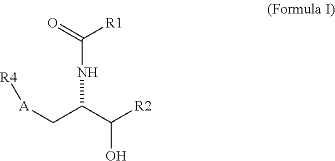
wherein R1 is a linear or branched C₁-C₂₇ alkane or C₂-C₂₇ alkene; or R1 is —C(OH)—R3 wherein R3 is a linear or branched C₁-C₂₆ alkane or C₂-C₂₆ alkene; or R1 is a C₆-C₂₇ alkane or alkene wherein (i) the C₆-C₂₇ alkane or alkene is substituted with a C₅-C₁₅ cycloalkane, C₅-C₁₅ cycloalkene, heterocycle, or aromatic ring or (ii) the C₆-C₂₇ alkane or alkene includes, within the C₆-C₂₇ alkyl or alkenyl chain, a C₅-C₁₅ cycloalkane, C₅-C₁₅ cycloalkene, heterocycle, or aromatic ring;
R2 is one of the following (a)-(e):
—CH₂(CH₂)ₓCH₃, (a)
—CH(OH)(CH₂)ₓCH₃, (b)
—CH(OH)(CH₂)ₓCH(CH₃)₂, (c)
—CH═CH(CH₂)ₓCH₃, (d)
—CH(OH)(CH₂)ₓCH(CH₃)CH₂CH₃, (e)
wherein X is an integer ranging from 4-17;
R4 is an α-linked or a β-linked monosaccharide, or when R1 is a linear or branched C₁-C₂₇ alkane, R4 is: 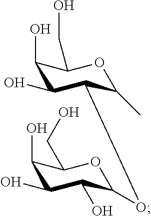
and
A is O or —CH₂.

69. The method of claim 68, wherein R1 is —(CH₂)₂₂CH₃ or —(CH₂)₂₄—CH₃, wherein R2 is —CH(OH)—(CH₂)₁₃CH₃, or wherein R4 is galactosyl, mannosyl, fucosyl or glucosyl.

70-71. (canceled)

72. The method of claim 56, wherein said ceramide-like glycolipid comprises an α-galactosylceramide or an analog thereof.

73. The method of claim 72, wherein said α-galactosylceramide or analog thereof comprises Formula II: 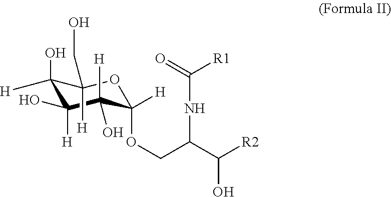
wherein
R1 is a linear or branched C₁-C₂₇ alkane or C₂-C₂₇ alkene; or R1 is —C(OH)—R3 wherein R3 is linear or branched C₁-C₂₆ alkane or C₂-C₂₆ alkene; and
R2 is one of the following (a)-(e):
—CH₂(CH₂)ₓCH₃, (a)
—CH(OH)(CH₂)ₓCH₃, (b)
—CH(OH)(CH₂)ₓCH(CH₃)₂, (c)
—CH═CH(CH₂)ₓCH₃, (d)
—CH(OH)(CH₂)ₓCH(CH₃)CH₂CH₃, (e)
wherein X is an integer ranging from 4-17.

74. The method of claim 73, wherein R2 is —CH(OH)(CH₂)ₓCH₃, wherein X is an integer ranging from 4-13.

75. The method of claim 73, wherein R1 is selected from the group consisting of (CH₂)₉CH═CH—CH₂—CH═CH(CH₂)₄CH₃, (CH₂)₈CH═CH—CH₂—CH═CH(CH₂)₄CH₃, (CH₂)₇CH═CH—CH₂—CH═CH(CH₂)₄CH₃, (CH₂)₃CH═CH—CH₂—CH═CH—CH₂—CH═CH—CH₂—CH═CH—(CH₂)₄CH₃, (CH₂)₃CH═CH—CH₂—CH═CH—CH₂—CH═CH—CH₂—CH═CH—CH₂—CH═CH—CH₂CH₃, (CH₂)₇CH═CH—CH₂—CH═CH═(CH₂)₄CH₃, (CH₂)₇CH═CH—CH═CH(CH₂)₅CH₃, (CH₂)₈CH═CH—CH═CH(CH₂)₄CH₃, (CH₂)₉CH═CH—CH═CH(CH₂)₅CH₃, (CH₂)₆CH═CH—CH═CH—CH═CH(CH₂)₄CH₃, (CH₂)₆CH═CH—CH═CH—CH═CH(CH₂)₄CH₃ and (CH₂)₇CH═CH—CH═CH—CH═CH(CH₂)₃CH₃.

76. The method of claim 75, wherein the double bonds are cis or trans.

77. The method of claim 76, wherein said α-galactosylceramide or analog thereof comprises Formula III: 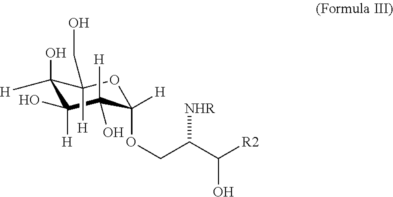
wherein R is H or —C(O)R1, wherein R1 is a linear or branched C₁-C₂₇ alkane or C₂-C₂₇ alkene; or R1 is —C(OH)—R3 wherein R3 is a linear or branched C₁-C₂₆ alkane or C₂-C₂₆ alkene; or R1 is a C₆-C₂₇ alkane or alkene wherein (i) the C₆-C₂₇ alkane or alkene is substituted with a C₅-C₁₅ cycloalkane, C₅-C₁₅ cycloalkene, heterocycle, or aromatic ring or (ii) the C₆-C₂₇ alkane or alkene includes, within the C₆-C₂₇ alkyl or alkenyl chain, a C₅-C₁₅ cycloalkane, C₅-C₁₅ cycloalkene, heterocycle, or aromatic ring; or R1 is a —(CH₂)ₙR5, wherein n is an integer ranging from 0-5, and R5 is —C(O)OC₂H₅, an optionally substituted C₅-C₁₅ cycloalkane, an optionally substituted aromatic ring, or an aralkyl, and
R2 is one of the following (a)-(e):
—CH₂(CH₂)ₓCH₃, (a)
—CH(OH)(CH₂)ₓCH₃, (b)
—CH(OH)(CH₂)ₓCH(CH₃)₂, (c)
—CH═CH(CH₂)ₓCH₃, (d)
—CH(OH)(CH₂)ₓCH(CH₃)CH₂CH₃, (e)
wherein X is an integer ranging from 4-17.

78. The method of claim 73, wherein R1 is substituted with oxo; hydroxy; halogen; phenyl; —OC(O)R6; —OR6; —C(O)R6; or N(R6)₂,
wherein each R6 is independently hydrogen, C₁-C₆ alkyl, or an aromatic ring optionally substituted with halogen; hydroxy; —OC(O)R7; —OR7; —C(O)R7 or N(R7)₂, and
wherein each R7 is independently hydrogen or C₁-C₆ alkyl, or wherein R1 is selected from the group consisting of 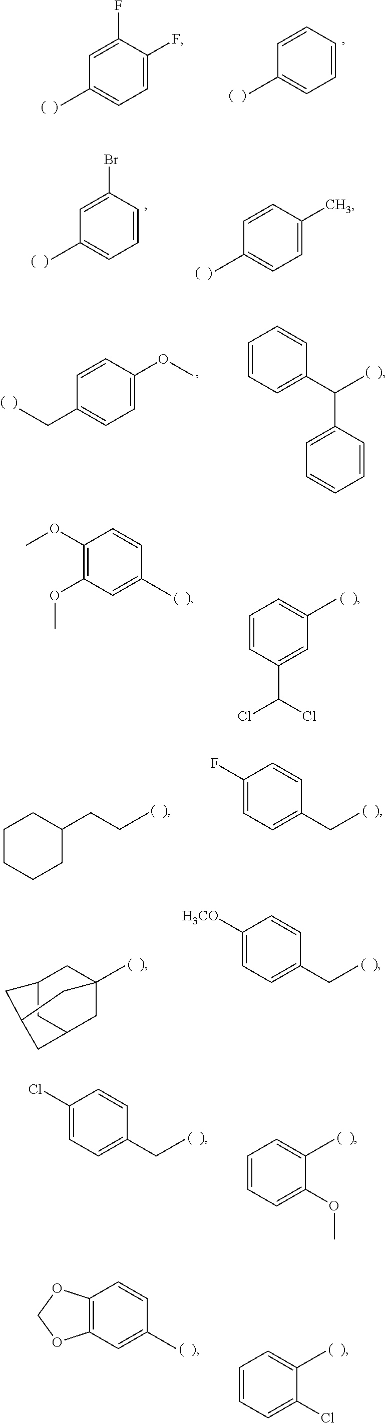 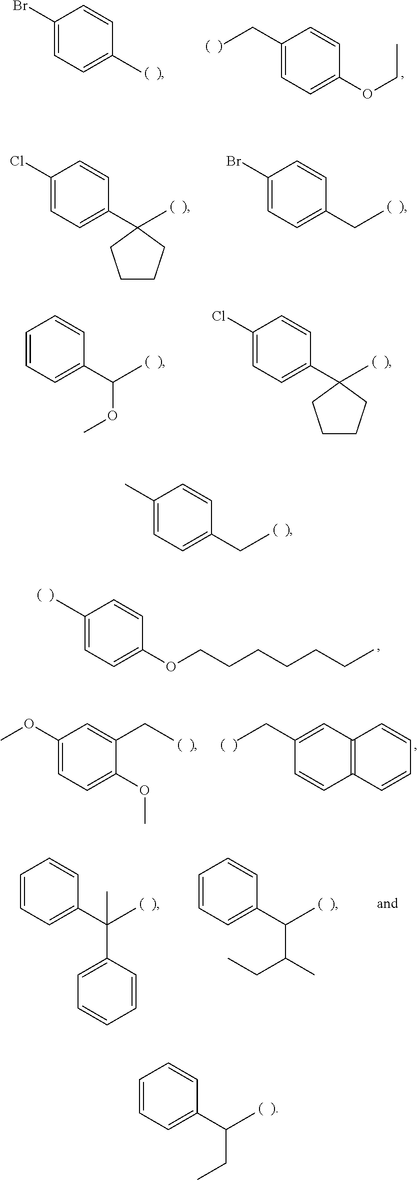
where ( ) represent the point of attachment of R1 to the compound of Formula III.

79. (canceled)

80. The method of claim 73, wherein said α-galactosylceramide or analog thereof comprises (2S,3S,4R)-1-O-(α-D-galactopyranosyl)-N-hexacosanoyl-2-amino-1,3,4-octadecanetriol (KRN7000), (2S,3S)-1-O-(α-D-galactopyranosyl)-N-hexacosanoyl-2-amino-1,3-octadecanediol), or (2S,3S,4R)-1-CH₂-(α-galactopyranosyl)-N-hexacosanoyl-2-amino-1,3,4-octadecanetriol (α-C-GalCer).

81. (canceled)

82. The method of claim 56, wherein said mycobacterium comprises a heterologous antigen.

83. The method of claim 82, wherein said heterologous antigen is expressed on the surface of said mycobacterium cell.

84. The method of claim 83, wherein said heterologous antigen is a viral antigen, a bacterial antigen, a fungal antigen, a parasitic antigen, or a tumor specific antigen.

85. (canceled)

86. The method of claim 84, wherein said heterologous antigen is an immunogenic peptide.

87. (canceled)

88. The method of claim 56, wherein said ceramide-like glycolipid is not a native lipid of the cell wall of said mycobacterium.

89. The method of claim 56, wherein said ceramide-like glycolipid stimulates natural killer T (NKT) cells.

90. The method of claim 82, wherein said heterologous antigen is chemically or physically conjugated to the surface of said mycobacterium.

91. The modified bacterium of claim 1, wherein said ceramide-like glycolipid is not a native lipid of the cell wall of said bacterial cell.

92. The modified bacterium of claim 1, wherein said ceramide-like glycolipid stimulates natural killer T (NKT) cells.

93. The modified bacterium of claim 1, wherein said heterologous antigen is chemically or physically conjugated to the surface of the bacterial cell.
